(19) United States
(12) Patent Application Publication
(10) Pub. No.: US 2007/0190534 A1
(43) Pub. Date: Aug. 16, 2007

(54) Mitochondrial sites and genes associated with prostate cancer

(75) Inventors: Mark Birch-Machin, Gosforth (GB); Gabriel D. Dakubo, Thunder Bay (CA); Robert Thayer, Thunder Bay (CA); Ryan Parr, Thunder Bay (CA)

Correspondence Address: TORYS LLP, 79 WELLINGTON ST. WEST, SUITE 3000, TORONTO, ON, M5K 1N2, CA

(73) Assignee: Genesis Genomics Inc.

(21) Appl. No.: 11/339,751

(22) Filed: Jan. 26, 2006

Related U.S. Application Data
(63) Continuation-in-part of application No. PCT/CA04/02124, filed on Dec. 13, 2004.
Application No. PCT/CA04/02124 is a continuation-in-part of application No. 10/732,374, filed on Dec. 11, 2003.
Application No. 10/732,374 is a continuation-in-part of application No. PCT/CA02/00848, filed on Jun. 10, 2002.
(60) Provisional application No. 60/646,588, filed on Jan. 26, 2005.
(60) Provisional application No. 60/297,340, filed on Jun. 11, 2001.

(51) Int. Cl. C12Q 1/68; G06F 19/00; C07H 21/04

(52) U.S. Cl. 435/6; 435/287.2; 702/20; 536/23.2

(57) ABSTRACT

A cluster analysis of mutations in the mitochondrial genomes from malignant, adjacent benign and distant benign tissues from a sample of men having prostate cancer has determined that mutations in ND1, ND2, COX1 and CytB are indicative of prostate cancer. 97% (30/31) of the samples contained synonymous and non-synonymous mutations in these genes. 65% (20/31) of the samples contained non-synonymous mutations in these genes.

### CROSS REFERENCE TO RELATED APPLICATIONS

This application claims priority to U.S. 60/646,588 filed Jan. 26, 2005. This application is also a continuation-in-part of PCT/CA04/02124 filed Dec. 13, 2004, which is a continuation-in-part of U.S. Ser. No. 10/732,374 filed Dec. 11, 2003, which is a continuation-in-part of PCT/CA02/00848 filed Jun. 10, 2002, which claims priority to U.S. 60/297,340 filed Jun. 11, 2001. All earlier applications are herein incorporated by reference.

### SEQUENCE LISTING RECORDED ON COMPACT DISC INCORPORATED BY REFERENCE

This invention incorporates by reference all materials recorded in ONE compact disc labeled COPY 1, and duplicate thereof labeled COPY 2. The material recorded on the compact disc is the Sequence Listing contained in the file entitled “seq.listing.txt” created Jan. 25, 2006 and having 766 KB. The material in COPY 1 and COPY 2 is identical. Applicant also submits an identical computer readable form (CFR COPY 3) on compact disc. The material in the CFR COPY 3 is identical to the material in COPY 1 and COPY 2.

### TECHNICAL FIELD OF THE INVENTION

This invention is related to the field of mitochondrial genomics. In particular it is related to mutations in the mitochondrial genome and their utility as an indicator of the genesis of disease, for example detecting the presence of pre-neoplasia, neoplasia and progression towards potential malignancy even before common clinical symptoms are evident.

### BACKGROUND OF THE INVENTION

The current mega-trend in the biological sciences is the human genome project, and commercial exploitation of the data. However, there is an exceptional limitation to the use and implementation of this information as the data is not specific at the level of the individual. Incredibly the data is from only a few individuals, hardly representative of the variation present in human populations, rendering the data useful in general applications only. The staggering complexity of the human genome makes application on an individual basis impractical. To sequence completely one human nuclear genome the U.S. Department of Energy and the National Institute of Health have invested 2.5 billion dollars since 1988 (http://www.ornl.gov/hgmis/project/budget.html).

Mitochondrial Genome

The mitochondrial genome is a compact yet critical sequence of nucleic acid. The mitochondrial genome codes for enzyme subunits necessary for cellular respiration. Mitochondrial DNA, or “mtDNA”, is a minuscule genome of nucleic acid at 16,569 base pairs (bp) Anderson et al., 1981; Andrews et al., 1999) in contrast to the immense nuclear genome of 3.3 billion bp. Its genetic complement is astronomically smaller than that of its nuclear cell mate (0.0005%). However, individual cells carry anywhere from 10³ to 10⁴ mitochondria depending on specific cellular function (Singh and Modica-Napolitano 2002). Communication or chemical signalling, routinely occur between the nuclear and mitochondrial genomes (Sherratt et al., 1997). Moreover, specific nuclear components are responsible for maintenance and integrity of mitochondrial sequence (Croteau et al., 1999). When these nuclear areas are rendered non-functional by nuclear rearrangements indicative of potential disease, then mutations begin to appear in mtDNA sequences. In addition, specific mitochondria may be identified for intracellular destruction by deletions prompted by somatic mutations in the mitochondrial genome. This theoretical mechanism may serve as an indication of impending disease as well. About 3,000 genes are required to make a mitochondrion, with only thirty-seven of these coded by the mitochondrial genome, indicating heavy mitochondrial dependence on nuclear loci (Naviaux, 1997).

All mitochondrial DNA (mtDNA) genomes in a given individual are identical given the clonal expansion of mitochondria within the ovum, once fertilization has occurred. The essential role of mtDNA is the generation of the cellular fuel, adenosine triphosphate (ATP), which fires cellular metabolism. Significantly, the mitochondrial genome is dependent on seventy nuclear encoded proteins to accomplish the oxidation and reduction reactions necessary to this vital function, in addition to the thirteen polypeptides supplied by the mitochondrial genome (Leonard and Shapira, 1997). Different tissues and organs depend on oxidative phosphorylation to a varied extent. Diseases related to defective oxidative phosphorylation (OXPHOS) appear to be closely linked to mtDNA mutations (Byrne, 1992). Consequently as OXPHOS diminishes due to increased severity of mtDNA mutations, organ specific energetic thresholds are not attained which give rise to a variety of clinical phenotypes. Moreover, mutations in the mitochondrial genome are associated with a variety of chronic, degenerative diseases (Gattermann et al. 1995). It is well known that aging and specific types of pathology can alter, or mutate mtDNA compromising the energy production capacity of the cell. This often results in over-expression of defective mitochondria, and/or the cell supplementing the lack of ATP by becoming more glycolytic (Carew and Huang, 2002); therefore, changes or mutations, in the mitochondrial genome can be used as markers for disease genesis and/or disease progression, when monitored at successive intervals.

Recently, Fliss et al. (2000) found, in primary tumours from lung and bladder cancer, a high frequency of mtDNA mutations which were predominantly homoplasmic in nature, indicating that the mutant mtDNA was dominant in the malignant cells. Point mutations and deletions would appear to be the non-programmed but unavoidable side effect of oxygen free radical damage to the membrane and genome of mitochondria (Miquel et al. 1992). This theory is plausible because not only is the mitochondrial genome lacking protective histones, but also is vulnerable to oxidative damage being found near the oxygen generating inner mitochondrial membrane. Moreover, as mtDNA has a compact genome and lacks introns, deleterious events are thus likely to affect a coding sequence resulting in a biochemical dysfunction. This dysfunction will further increase cellular oxidative stress which will lead to nuclear as well as mtDNA damage, thereby increasing the potential for a cell to enter into the cancer process (Penta et al., 2001). In this respect, research indicates that with increasing age there is an increase in mtDNA damage (Cortopassi & Wang 1995) and a subsequent decline in respiratory function (Miquel et al. 1992) leading to eventual cell death.

MtDNA as a Diagnostic Tool

MtDNA sequence dynamics are important diagnostic tools. Mutations in mtDNA are often preliminary indicators of developing disease, often associated with nuclear mutations, and act as biomarkers specifically related to disease, such as but not limited to: tissue damage and cancer from smoking and exposure to second hand tobacco smoke (Lee et al., 1998; Wei, 1998); longevity, based on accumulation of mitochondrial genome mutations beginning around 20 years of age and increasing thereafter (von Wurmb, 1998); metastatic disease caused by mutation or exposure to carcinogens, mutagens, ultraviolet radiation (Birch-Machin, 2000); osteoarthritis; cardiovascular, Alzheimer, Parkinson disease (Shoffner et al., 1993; Sherratt et al., 1997;Zhang et al, 1998); age associated hearing loss (Seidman et al., 1997); optic nerve degeneration and cardiac dysrhythmia (Brown et al., 1997; Wallace et al., 1988); chronic progressive external exophthalmoplegia (Taniike et al., 1992); atherosclerosis (Bogliolo et al., 1999); papillary thyroid carcinomas and thyroid tumours (Yeh et al., 2000); as well as others (e.g. Naviaux, 1997; Chinnery and Tumbull, 1999;).

Mutations at specific sites of the mitochondrial genome can be associated with certain diseases. For example, mutations at 4216, 4217 and 4917 are associated with Leber's Hereditary Optic Neuropathy (LHON) (Mitochondrial Research Society; Huoponen (2001); MitoMap). A mutation at 15452 was found in 5/5 patients to be associated with ubiquinol cytochrome c reductase (complex III) deficiency (Valnot et al. 1999). However, mutations at these sites were not found to be associated with prostate cancer.

Specifically, these alterations include point mutations (transitions, transversions), deletions (one base to thousands of bases), inversions, duplications, (one base to thousands of bases), recombinations and insertions (one base to thousands of bases). In addition, specific base pair alterations, deletions, or combinations thereof are associated with early onset of prostate, skin, and lung cancer, as well as aging (e.g. Polyak et al., 1998), premature aging, exposure to carcinogens (Lee et al., 1998), etc.

Since mtDNA is passed to offspring exclusively through the ovum, it is imperative to understand mitochondrial sequences through this means of inheritance. The sequence of mtDNA varies widely between maternal lineages (Ward et al., 1991), hence mutations associated with disease must be clearly understood in comparison to this variation. For example, a specific T to C transition noted in the sequence of several individuals, associated with a specific cancer, could in reality be natural variation in a maternal lineage widespread in a given particular geographical area or associated with ethnicity. For example, Native North Americans express an unusually high frequency of adult onset diabetes. In addition, all North American Natives are genetically characterized by five basic maternal lineages designated A, B, C, D, and X (Schurr et al., 1990; Stone and Stoneking, 1993; Smith et al., 1999). Lineage A is distinguished by a simple point mutation resulting in a Hae III site at bp 663 in the mitochondrial genome, yet there is no causative relationship between this mutation and the adult onset of diabetes. In addition, even within lineage clusters there is sequence variation.

Outside of the specific markers associated with a particular lineage there is more intrapopulation variation than interpopulation sequence variation (Easton et al., 1996; Ward et al., 1991, 1993;) This divergence must be understood for optimal identification of disease associated mutations, hence a maternal line study approach (Parsons et al., 1997), mimicking the strengths of a longitudinal design (i.e. subject tracking over a substantial period of time), must be used to identify mutations directly associated with disease, as opposed to mutations without disease association. Moreover, particular substances, such as second hand tobacco smoke, low levels of asbestos, lead, all known mutagens and at low levels in many environments, may be the cause of specific point mutations, but not necessarily a disease specific marker. Hence, a substantial mtDNA sequence database is a clear prerequisite to accurate forecasting of potential disease as a natural process, or through exposure to causative agents. Furthermore, the entire molecule must be sequenced for its full information content. The entire suite of point mutations (transitions, transversions), deletions (one base to thousands of bases), inversions, duplications, (one base to thousands of bases), recombinations and insertions (one base to thousands of bases) must be characterized as a whole over the entire mitochondrial genome. This ensures that all possible information available in the mitochondrial genome is captured. Although the genome of cytoplasmic mitochondria (16,569 bp) has been sequenced at an individual level, like its nuclear counterpart, the mitochondrial genome has not been sequenced at a population level for use as a diagnostic tool.

Recently mitochondria have been implicated in the carcinogenic process because of their role in apoptosis and other aspects of tumour biology (Green & Reed, 1998, Penta et al., 2001), in particular somatic mutations of mtDNA (mtDNA) have been observed in a number of human tumours (Habano et al. 1998; Polyak et al. 1998; Tamura et al. 1999; Fliss, et al. 2000). These latter findings were made more interesting by the claims that the particular mtDNA mutations appeared to be homoplasmic (Habano et al. 1998; Polyak et al. 1998; Fliss, et al. 2000). Additionally researchers have found that ultraviolet radiation (UV) is important in the development and pathogenesis of non-melanoma skin cancer (NMSC) (Weinstock 1998; Rees, 1998) and UV induces mtDNA damage in human skin (Birch-Machin, 2000a).

Moreover, through time, mitochondrial sequence loses integrity. For example, the 4977 bp deletion increases in frequency with age (Fahn et al., 1996). Early in life, this deletion begins to occur in small numbers of mitochondria. By age 80, a substantial number of molecules have been deleted. This deletion characterizes the normal aging process, and as such serves as a biomarker for this process. Quantification of this aging process may allow medical or other interventions to slow the process.

This application of mitochondrial genomics to medicine has been overlooked because mtDNA has been used primarily as a tool in population genetics and more recently in forensics; however, it is becoming increasingly evident that the information content of mtDNA has substantial application in the field of medical diagnostics. Moreover, sequencing the entire complement of mtDNA was a laborious task before the recent advent of high capacity, high-throughput robotic DNA sequencing systems. In addition, population geneticists were able to gather significant data from two highly variable areas in the control region; however, these small regions represent a small portion of the overall genome, less than 10%, meaning that 90% of the discriminating power of the information is left unused! Significantly, many disease associated alterations are outside of the control region. The character of the entire genome should be considered to include all sequence information for accurate and highly discriminating diagnostics.

Non-Melanoma Skin Cancer

Human non-melanoma skin cancer (NMSC) is the commonest cancer in many Caucasian populations (Weinstock, 1998; Rees, 1998). The majority of these tumours are basal cell carcinoma (BCC) and squamous cell carcinoma (SCC). BCCs are locally invasive and can cause significant morbidity but rarely metastasize. SCCs show significant metastatic potential and the occurrence of multiple NMSCs in patients with immunosuppression causes significant management problems (Rees, 1998). While there are no clinically identified pre-malignant lesions for BCC, some SCCs are thought to arise from precursor lesions, namely actinic keratoses (AKs) or areas of Bowen's disease (in situ carcinoma) (Rees, 1998).

SCCs show loss of heterozygosity affecting several chromosomes which suggests the involvement of several tumour suppressor genes in their development. Interestingly, in AKs, an equal or greater degree of genetic loss is observed in these precursor lesions compared to SCCs (Rehman et al. 1994; Rehman et al. 1996). This is important for the proposed invention because it suggests that other mechanisms, in addition to inactivation of tumour suppressor genes, are likely to be involved in the development of SCCs.

A role for mitochondria in tumourigenesis was originally hypothesised when tumour cells were found to have an impaired respiratory system and high glycolytic activity (Shay & Werbin, 1987). Recent findings elucidating the role of mitochondria in apoptosis (Green & Reed, 1998) together with the high incidence of homoplasmic mtDNA mutations in colon cancer (Habano et al. 1998; Polyak et al. 1998, reviewed in Penta et al., 2001), primary tumours of the bladder, neck and lung (Fliss et al. 2000), and gastric tumours (Tamura et al. 1999), further support this hypothesis. Furthermore, it has been proposed that these mitochondrial mutations may affect the levels of reactive oxygen species (ROS) which have been shown to be highly mitogenic (Polyak et al. 1998; Li et al. 1997).

Previous studies by the inventors and others have shown that mutations in mtDNA and the associated mitochondrial dysfunction is an important contributor to human degenerative diseases (Birch-Machin et al. 1993; Chinnery et al. 1999; Birch-Machin et al. 2000b). This is because the mitochondrial genome is particularly susceptible to mutations due to the high amounts of ROS produced in this organelle coupled with the lack of protective histones and a low rate of mtDNA repair (Pascucci et al. 1997; Sawyer & van Houten; LeDoux et al. 1999) compared to the nucleus. Indeed, the mutation rate for mtDNA is around ten times higher than that of nuclear DNA (Wallace, 1994). Most of the mtDNA mutations identified in the recent human tumour studies have indicated possible exposure to ROS derived mutagens. This is important for the investigation of mtDNA mutations in NMSC because there is recent evidence for the direct involvement of UV induced ROS in the generation of mtDNA deletions in human skin cells (Berneburg et al. 1999, Lowes et al., 2002). In addition, the major determinant of NMSC in individuals without protective pigmentation or genetic predisposition is UV (Weinstock, 1998). The putative precursor lesions of SCCs are also found predominantly on constant sun-exposed sites. This is important because work by the Birch-Machin laboratory has shown distinct differences between the incidence of mitochondrial DNA damage in skin taken from different sun-exposed body sites. The vast majority of the damage is found on constant sun-exposed sites (Krishanan et al., 2002).

One of the inventors was the first to quantitatively show that UV exposure induces mtDNA damage (Birch-Machin et al. 1998). MtDNA as a molecular marker was used to study the relation between chronological aging and photo aging in human skin. A 3-primer quantitative PCR method was used to study the changes in the ratio of the 4977 bp-deleted to wild type mtDNA in relation to sun exposure and chronological age of human skin. There was a significant increase in the incidence of high levels (i.e. >I %) of the 4977 bp-deleted mtDNA in sun-exposed (27%, [ 27/100]) compared with sun-protected sites (1.1% [ 1/90]) (Fishers exact test, P<0.0001). Deletions or mutations of mtDNA may therefore be useful as a marker of cumulative ultraviolet radiation exposure.

Furthermore, a study using a South-Western Blot approach involving monoclonal antibodies against thymine dimers, provided direct evidence for the presence of UV-induced damage in purified mtDNA (Ray et al. 1998).

Quantification of a single deletion alone, however, may not provide a reliable UV bio-marker because it represents one of many possible deletions or combinations and other associated mutations (Birch-Machin, 2000). Recent work from the inventors' research group has used a long extension PCR (LX-PCR) technique to amplify the entire mitochondrial genome in order to determine the whole deletion spectrum of mtDNA secondary to UV exposure (Ray et al. 2000). Long PCR analysis of 71 split skin samples, where the epidermis is separated from the underlying dermis, was performed in relation to sun exposure. There was a significant increase in the number of deletions with increasing UV exposure in the epidermis (Kruskal-Wallis test, p=0.0015). The findings in the epidermis are not confounded by any age-dependent increases in mtDNA deletions also detected by the long PCR technique. The large spectrum of identified deletions highlights the ubiquitous nature and the high mutational load of mtDNA associated with UV exposure. Compared to the detection of single deletions using competitive PCR, the study shows that long PCR is a sensitive technique and may therefore provide a more comprehensive, although not quantitative, index of overall mtDNA damage in skin. The studies by one of the inventors described above clearly show that mtDNA is a significant target of UV and this together with the role of mitochondrial in skin disease has been recently reviewed (Birch-Machin, 2000).

The pigmentation of human hair and skin which is the major co-variant of UV sensitivity and human skin cancer has been investigated. These investigations have centred on the association of variants of the melanocortin 1-receptor gene and sun-sensitivity of individuals and populations (Smith et al. 1998; Healy et al. 1999; Flanagan et al. 2000; Healy et al. 2000; Harding et al. 2000; Flanagan et al., 2002) relating to skin cancer susceptibility. However, these studies have not addressed population-level variation in mtDNA sequences in association with particular skin types and/or hair colour.

One of the questions which remains largely unanswered by the recent studies of mtDNA mutations in human tumours is the incidence of deletions of the mitochondrial genome in relationship to these tumours. This is an important question to answer because a preliminary study of a single patient in human skin has shown differences in the incidence of the common mtDNA deletion between several tumours (AKs and SCCs) and normal skin (Pang et al. 1994). As well, the inventors' own preliminary data shows an increased number of mtDNA deletions in tumours compared to normal skin. Finally, Birch-Machin and others have shown that the incidence of mtDNA deletions, as well as duplications, increases with increasing UV exposure (Berneburg et al. 1999; Birch-Machin et al. 1998; Ray et al. 1998; Ray et al. 1999; Ray et al. 2000), Lindsey et al., 2001; Birch-Machin et al., 2001; Lowes et al., 2002, Krishnan et al., 2002).

Apart from the questions relating to tumour progression other vital questions remain largely unanswered by the recent studies of mtDNA in human tumours (Habano et al. 1998; Fiiss et al. 2000). Firstly, due to technical limitations, it is not clear whether the mtDNA mutations are truly homoplasmic, as varying levels of heteroplasmy may indicate important disease transitions as well (Habano et al. 1998; Polyak et al. 1998; Fliss, et al. 2000); secondly, apart from one study (Tamura et al. 1999) the incidence of mtDNA deletions and their role as potential biomarkers for NMSC was not investigated. Researchers have looked at the common deletion and ignored the rest of the 100 or so deletions. As well, investigators have been focused on identification of mutations, rather than their quantification. It is important to assess accurately in a quantitative manner the incidence of deletions because of the threshold effect of mtDNA damage on ATP production and consequently cell function. In addition, deletions are difficult to characterize. Long PCR is typically used which produces a ladder of deletions which then have to be characterized.

Current diagnosis of NMSC is pathological evaluation of excised tissue. Accordingly, there is a need for an early marker of UV-induced DNA damage which predisposes an individual to NMSC. There is also a need for a genetic-based diagnostic tool which allows for early detection and is diagnostically accurate.

Prostate Cancer

Prostate cancer is a frequently diagnosed solid tumour that most likely originates in the prostate epithelium (Huang et al. 1999). In 1997, nearly 10 million American men were screened for prostate specific antigen (PSA), which at increased levels is indicative of prostate cancer (Woodwell, 1999). An even greater number of men are screened by an initial digital rectal exam (DRE). In the same year, 31 million men had a DRE (Woodwell, 1999). Moreover, the annual number of newly diagnosed cases of prostate cancer in the United States is estimated at 179,000 (Landis et al., 1999). It is the second most commonly diagnosed cancer and second leading cause of cancer mortality in Canadian men. In 1997 prostate cancer accounted for 19,800 of newly diagnosed cancers in Canadian men (28%) (National Cancer Institute of Canada). It is estimated that 30% to 40% of all men over the age of forty-nine (49) have some cancerous prostate cells, yet only 20% to 25% of these men have a clinically significant form of prostate cancer (SpringNet—CE Connection, internet, www.springnet.com/ce/j803a.htm). Prostate cancer exhibits a wide variety of histological behaviour involving both erogenous and exogenous factors, i.e. socio-economic situations, diet, geography, hormonal imbalance, family history and genetic constitution (Konishi et al. 1997; Hayward et al. 1998).

From a risk standpoint familial and hereditary prostate cancers are not considered synonymous terms. Familial cancers refer to the incidences within a family, but are not inherited. This form accounts for up to 25% of prostate cancers (Walsh & Partin, 1997). Hereditary refers to a subtype of prostate cancer with a Mendelian inheritance of a predisposing gene(s) and accounts for approximately 9% of reported cases. A positive family history of prostate cancer for this disease suggests that these predisposing gene(s) play an important role in prostate cancer development and progression. Recently, susceptibility genes on chromosomes 1 and X have been identified as predisposing men to prostate cancer, providing greater insight into the etiology of hereditary cancer (Berthon et a]. 1998; Xu et al. 1998).

Prostate cancer prognosis mainly depends on the tumour stage and grade at diagnosis. Only localized prostate cancer can be cured by radical treatment. Standard detection still relies on digital rectal examination, PSA testing and histopathologic examination of prostatic biopsied tissues. Biopsy of a mass is used to confirm malignancy, it is not an early detection technique. Unfortunately, some early tumours are impossible to identify during rectal exams. PSA tests have a specificity of 60 to 70% and a sensitivity of 70 to 80% (personal communication, Dr. Sunil Gulavita, Northwestern Ontario Cancer Centre). A newer technique which refines diagnosis for tumours of common histologic grade is ploidy-DNA analysis employing flow cytometry (Shankey et al. 1995); however, this technique measures chromosomal changes that are only apparent in later stages of cancer development and is not sufficiently sensitive for the detection of minor alterations in DNA structure or chromosomal inversions, or reciprocal trans-locations in early cancers. The invention focuses on early detection since prognosis is heavily dependent on the stage of disease at diagnosis.

Our understanding of genetic abnormalities in prostate cancers is scanty. Research into prostate cancer has focussed on the development of knowledge in the following areas: 1) proto-oncogenes (Buttyan et al. 1987); 2) tumour suppressor genes (p53, p73, KAI1 and MMACI/PTEN; Dong et al. 1995; Cairns et al. 1997) and 3) telomere/telomerase activity in metastasis. Up-regulation of telomerase and amplification of telomeric DNA in prostate cells may provide effective markers for diagnosis. Moreover, telomeres may serve as a site for therapy (Ozen et al. 1998). A number of groups have provided evidence for a “prostate cancer gene” in the short arm of chromosome 1 (Berthon et al. 1998). More work is needed to identify the specific locus within this region. It has been suggested that this marker is only one of several possible genes predisposing men to familial prostate cancer. Other studies have shown possible marker loci on the X chromosome (Xu et al. 1998). If some prostate cancers are polygenic, then mtDNA becomes an important diagnostic tool since it may be difficult to identify and understand the interplay between all associated nuclear genes in such cases.

Certainly, a key issue in prostate cancer research is to identify molecular markers that can effectively determine and distinguish tumour progression. Molecular markers may be able to discriminate between those cases of prostate neoplasmy which will proceed rapidly to metastatic disease and those with little chance of resulting in tumour development. Comparison of molecular markers or mutations can determine whether the tumour pathway is latent or aggressive. Up to the present research has focused primarily on the secrets hidden within the nuclear genome; however, the much smaller mtDNA genome seems to act as a barometer for events in the nucleus and as such provides a means for the early detection of human prostate cancer (Zeviani et al. 1990). Importantly, in this respect, mitochondria have been implicated in the carcinogenic process because of their role in apoptosis and other aspects of tumour biology (Green & Reed 1998). In particular, somatic mutations of mtDNA have been observed in a number of human tumours (Polyak et al. 1998, Tamura et al. 1999, Fliss et al. 2000). However, previous studies have been exclusively cross-sectional as they have not considered the clonal nature of mtDNA in maternal lines. These limited cross-sectional studies merely show the mutation at one time point. This may or may not give an accurate link between a mutation and the corresponding disease state. Cross-sectional studies employing a maternal line have the advantage of tracking a mutation in mtDNA over time and thus mimic the strength of a longitudinal design. Mutations which are common population variants, as opposed to mutations associated with disease, can both be identified.

Aging

Aging consists of an accumulation of changes with time both at the molecular and cellular levels; however, the specific molecular mechanisms underlying the aging process remain to be elucidated. In an attempt to explain the aging process, mitochondrial genomes in older subjects are compared to the genomes of younger subjects from the same maternal lineage. One deletion associated with aging is known as the common deletion, or 4977-bp deletion. Aging research has been limited to this common deletion and polymorphisms in the control region. For a clear understanding of these mutations, the entire genome must be analyzed. Other deletions are seen in Table 1 adapted from Wei, 1992.

TABLE 1

| Deletions | |
|---|---|
| size (bp) | References |
| 4977 | Cortopassi and Arnheim, 1990; |
| | Ikebe et al., 1990; Linnane et al., 1990; |
| | Corral-Debrinski et al., 1991; Yen et al., 1991; |
| | Torii et al., 1992; Zhang et al., 1992 |
| 7436 | Corral-Debrinski et al., 1991; |
| | Hattori et al., 1991 |
| | Hsieh and Wei, 1992 |
| 3610 | Katayama et al., 1991 |
| 6063 | Hsieh and Wei, 1992 |
| | Yen et al., 1992 |
| 5827 | Zhang et al., 1992 |
| 6335 | Zhang et al., 1992 |
| 7635 | Zhang et al., 1992 |
| 7737 | Zhang et al., 1992 |
| 7856 | Zhang et al., 1992 |
| 8041 | Zhang et al., 1992 |
| 8044 | Zhang et al., 1992 |
| 5756 | Zhang et al., 1992 |

Oxygen free radicals, a normal by product of ATP production, are a probable cause of this deletion, which increases in frequency with age. Existing literature demonstrates a strong association between mtDNA (mtDNA) mutations, chronological age, and the overall aging process in postmitotic tissues such as muscle and brain; however, comparative maternal line studies are needed to discriminate between aging assiociated mutational events and those mutations without an aging association.

In recent years a variety of chronic degenerative diseases have been shown to result from mutations in mtDNA (Gatterman et al. 1995). Diseases related to defective OXPHOS appear to be mutations in mtDNA mutations (Byrne, 1992). Furthermore, it has been shown that these myopathies are often associated with the common deletion of 4977-bp of the mitochondrial genome (Liu et al. 1997). This large deletion has also been found, at heteroplasmic levels, in various tissues of normal aging persons and is consistent with the Mitochondrial Theory of Aging (Harman, 1981). This is manifest through an increase in the deletion frequency (Cortopassi & Wang, 1995) and a subsequent decline in respiratory function (Miquel et al. 1992) resulting in eventual cell death in old age. The early detection of a predisposition to a disease or disorder presents the best opportunity for medical intervention, as early genetic diagnosis may improve the prognosis for a patient.

Previous studies employing a cross-sectional design have established an association or cause and effect relationship between mtDNA mutations, deletions, and/or combinations of such and aging; however, in order to obtain accurate data the age specific deletion and/or mutation rate must be determined concisely. Attributing mutations to the aging process as opposed to a particular disease at the population level is vital. This information is imperative to an understanding of how mtDNA damage accrues over time. Moreover, the consequences of these particular mutations, their frequencies, and associations in the temporal aspects of aging must be known in order to forecast and eventually slow aging at the molecular level. Researchers have not yet determined this rate, which requires evaluation of population data through maternal lines. Accordingly, there is a need for a biomarker which tracks the aging process.

Accordingly, there is a need for a simple, straightforward system of monitoring the mitochondrial genome for mutations which indicate early stage cancer, aging or other human diseases with a DNA component. There is also a need for a simple diagnostic system for non-melanoma skin cancer, prostate cancer, lung cancer and aging linked to defects in the mitochondrial genome. There is a need for a diagnostic system which differentiates between mutations in mtDNA which cause disease, and those which simply represent variation within and between populations.

### SUMMARY OF THE INVENTION

An object of the present invention is to provide a simple, straightforward system for monitoring the mitochondrial genome for early transitions associated with cancer, aging, and other human diseases with a DNA component.

In an embodiment of the present invention a small biological sample which includes tissue or fluid samples such as urine, prostate fluid, skin cells, or saliva is taken from an individual. These samples are examined, using any suitable method including histological examination, to identify cells demonstrating disease morphology. Using any suitable method, including without limitation; laser capture, microdissected cells demonstrating disease morphology are recovered from the sample and the mtDNA therefrom is sequenced, followed by comparison to a database of known mitochondrial sequences associated with both health and disease.

In a preferred embodiment, the entire mitochondrial genome is sequenced at a population level to determine the variation of mtDNA sequences associated with disease.

In an additional embodiment, the presence of mutation progression may signal the beginning and continuing development of disease. Mutation load may also indicate progression or disease state.

In a preferred embodiment, mtDNA sequences from prostate massage fluid are compared to a mtDNA sequence database of normal, transitory, and metastatic mtDNA sequences clearly associated with prostate cancer. This comparative data set is based on studies of maternal lines, and other normal maternal line variation present in the population stored in a maternal line database affording a lucid picture of mtDNA mutations clearly associated with disease, as opposed to variation present in mitochondrial lineages existing in the general population.

There may be specific maternal lineages which indicate a predisposition to disease.

In another embodiment, mtDNA sequences from suspected non-melanoma skin cancers are compared to a mtDNA sequence database of normal and mtDNA sequences clearly associated with non-melanoma skin cancer.

According to an aspect of the present invention, there is provided a method of detecting in a subject containing mtDNA the genesis or progression of disease comprising obtaining a biological sample from the subject, extracting DNA from the biological sample, and detecting the presence of mutations in the mtDNA. The step of detecting the presence of mutations is chosen from sequencing the mtDNA, amplifying the mtDNA by PCR, South-Western blotting, denaturing HPLC, hybridization to microarrays, gene chips or biochips, molecular marker analysis or combinations thereof. Further, the mtDNA of the biological sample is compared to a database, the database containing data of mutations associated with the mtDNA sequences of non-disease and disease associated mitochondrial genomes.

According to an aspect of the present invention, there is provided a method of detecting in a human subject the presence of a disease comprising obtaining a biological sample from the human subject, extracting DNA from the biological sample, detecting mutations in the mitochondrial DNA of the biological sample, and comparing the mitochondrial DNA sequence of the biological sample to a database, the database containing data of mutations associated with the mitochondrial DNA sequences of non-disease and disease associated mitochondrial genomes Mutation rates of mitochondria DNA associated with a specific disease may be an important indicator of disease development and prognosis. This may allow specific identification of disease stage, improving disease definition resulting in better disease intervention and specific therapy application.

In yet another embodiment, increasing the sensitivity for heteroplasmy detection increases the early identification capacity of the test.

In addition, the invention may be used to monitor the progression of disease by watching important sites targeted by metastasis.

According to another aspect of the present invention, there is provided a method of determining a predisposition to a disease or disorder indicated by mutations in a mitochondrial DNA sequence comprising: obtaining a biological sample from the human subject, extracting DNA from the biological sample, detecting mutations in the mitochondrial DNA of the biological sample, and comparing the mitochondrial DNA sequence of the biological sample to a database, the database containing data of mutations associated with the mitochondrial DNA sequences of individuals who are predisposed to the disease or disorder, and individuals who are not predisposed to the disease or disorder.

In a preferred embodiment, a DNA microarray is used in determining the sequence of the mitochondrial DNA. Other technologies can also be used. For example, direct sequencing of a subset, or the complete human genome, SNaP shot™, SNP detection, real time PCR or other methods as is standard in the art.

According to a further aspect of the present invention, there is provided a method for assessing the status of the aging process of a human subject comprising obtaining a biological sample from the human subject, extracting DNA from the biological sample, detecting mutations in the mitochondrial DNA of the biological sample, and comparing the mitochondrial DNA sequence of the biological sample to a database, the database containing data of mutations of TDNA associated with aging.

The step of detecting the presence of mutations in the mtDNA can be selected from: sequencing the mtDNA, amplifying mtDNA by PCR, Southern, Northern, Western, South-Western blot hybridizations, denaturing HPLC, hybridization to microarrays, biochips or gene chips, molecular marker analysis, biosensors, melting temperature profiling or a combination of any of the above.

According to yet another aspect of the present invention, there is provided a database containing a plurality of human mitochondrial DNA sequences, the mitochondrial DNA sequences selected from the group of normal control sequences associated with non-disease states, sequences associated with the presence of disease or sequences indicative of the predisposition to disease.

According to yet another aspect of the present invention, there is provided a kit for diagnosis of a disease comprising a disposable chip, microarray, means for holding the disposable chip, means for extraction of mitochondrial DNA and means for access to a database of mitochondrial DNA sequences.

According to yet another aspect of the present invention there is provided a method of diagnosing a disease in a patient comprising hybridizing a nucleic acid sample obtained from mitochondrial DNA to an array comprising a solid substrate and a plurality of nucleic acid members, wherein each member is indicative of the presence of a disease, wherein each nucleic acid member has a unique position and is stably associated with the solid substrate, and wherein hybridization of said nucleic acid sample to one or more nucleic acid members comprising said array is indicative of the presence of prostate cancer.

According to yet another aspect of the present invention there is provided a kit for determining predisposition to a disease comprising a disposable chip, microarray, means for holding the disposable chip, means for extraction of DNA and means for access to a database of mitochondrial DNA sequences.

According to another aspect of the present invention, there is provided a method of determining a predisposition to or developing symptoms of a disease or disorder indicated by mutations in a mitochondrial DNA sequence comprising obtaining a biological sample from the human subject, extracting mitochondrial DNA from the biological sample, sequencing the mitochondrial DNA of the biological sample, and comparing the mitochondrial DNA sequence of the biological sample to a database, the database containing population-level data of mutations associated with the mtDNA sequences of non-disease and disease associated mitochondrial genomes.

According to yet another aspect of the present invention, there is provided a method of diagnosing non-melanoma skin cancer in a patient comprising: hybridizing a nucleic acid sample obtained from mitochondrial DNA to an array comprising a solid substrate and a plurality of nucleic acid members, wherein each member is indicative of non-melanoma cancer, wherein each nucleic acid member has a unique position and is stably associated with the solid substrate, and wherein hybridization of said nucleic acid sample to one or more nucleic acid members comprising said array is indicative of the presence of non-melanoma skin cancer. Alternatively, non-specific mutations may reach a threshold effect beyond which cancer develops. In a similar manner, prostate cancer can also be diagnosed.

According to another aspect of the present invention, there is provided a method of detecting heteroplasmy in a subject containing mtDNA comprising obtaining a biological sample from the subject; extracting DNA from the biological sample; and performing denaturing HPLC on the sample.

According to another aspect of the present invention, there is provided a method of detecting mutations associated with disease in a subject containing mtDNA comprising: obtaining a biological sample from the subject, extracting DNA from the biological sample, detecting the presence of mutations in the mtDNA, and comparing the mtDNA of the biological sample to a database, the database containing data of common population variants in non-disease and disease associated mitochondrial genomes.

Another aspect of the invention is to provide a use of any one or any combination of the mutations, substitutions, deletions or insertions listed on Table 4 or SEQ ID NOs: 102 to 138 to detect a predisposition to a disease or disorder, early detection of a disease or a disorder, genesis of a disease or a disorder, presence of a disease or a disorder, progression of a disease or a disorder, or aging in a subject having mtDNA.

Another aspect of the invention is to provide a method of detecting mutations associated with a disease, a disorder or aging in a subject having mtDNA, comprising: providing a biological sample from the subject, wherein the biological sample is chosen from non-involved tissue, distant benign tissue, adjacent benign tissue, atypical tissue, histologically abnormal tissue, and diseased tissue; extracting DNA from the biological sample; detecting the presence of mutations in the mtDNA; and determining whether the mutations are associated with normal interpopulation or intrapopulation variations, or whether the mutations are associated with the disease, the disorder or aging. Optionally, the method further comprises at least one of determining total mutation load in the mtDNA of the biological sample; and determining the identity of the mutation in the mtDNA of the biological sample. The step of determining whether the mutations are associated with normal interpopulation or intrapopulation variations, or whether the mutations are associated with the disease, the disorder or aging may comprise at least one of: comparing the mtDNA of the biological sample to a database, the database containing data of interpopulation and intrapopulation variations, and mutations associated with the disease, the disorder or aging; and determining total mutation load of the biological sample.

The diagnosis may be chosen from predisposition to a disease or a disorder, early detection of a disease or a disorder, genesis of a disease or a disorder, presence of a disease or a disorder, and progression of a disease or a disorder. The step of detecting the presence of mutations may be chosen from: sequencing the mtDNA; amplifying mtDNA by PCR; Southern, Northern, Western and South-Western blot hybridizations; denaturing HPLC; hybridization to microarrays, gene chips or biochips; molecular marker analysis; and a combination of any of the above.

The disease may be non-melanoma skin cancer or prostate cancer. The database contains at least a statistically significant number of mitochondrial DNA sequences, the mitochondrial DNA sequences having been obtained from a maternal line, a non-maternal line, or both.

Another aspect of the invention is to provide an array comprising a plurality of nucleic acid members, and a solid substrate, wherein the nucleic acid members are associated with the mutations listed on Table 4 or SEQ ID Nos: 102 to 138 and are indicative of the presence or predisposition of a disease, a disorder or aging, or used to determine a prohibiting index by quantifying the proportion of base pair deletions and mutations associated with a disease, a disorder or aging, and is chosen from mitochondrial DNA, RNA transcribed from mitochondrial DNA, and cDNA, wherein each nucleic acid member has a unique position on said array and is stably associated with the solid substrate. For example, the members may be associated with prostate cancer or any other disease or disorder.

Another aspect of the invention is to provide a kit for diagnosing, determining a predisposition, or early detection of a disease comprising a disposable chip, the array described above, means for holding the disposable chip, means for extraction of mitochondrial DNA and means for access to a database of mitochondrial DNA sequences. For example, the member may be associated with prostate cancer.

Another aspect of the invention is to provide a database containing a plurality of human mitochondrial DNA sequences, the mitochondrial DNA sequences are chosen from normal control sequences associated with non-disease states, sequences associated with interpopulation variations, sequences associated with intrapopulation variations, and sequences associated with the mutations on Table 4 or SEQ ID Nos: 102 to 138.

Another aspect of the invention, is a method of monitoring a person for the presence of pre-neoplasia, neoplasia or progression of neoplasia toward potential malignancy, in a biological sample, comprising (a) providing a biological sample from the subject, (b) extracting DNA from the biological sample, (c) detecting the presence of mutations in the mtDNA, (d) determining whether the mutations are associated with normal interpopulation or intrapopulation variations, or whether the mutations are associated with pre-neoplasia, neoplasia or progression of neoplasia toward potential malignancy, and (e) repeating steps (a) through (d). The step of determining whether the mutations are associated with pre-neoplasia, neoplasia or progression of neoplasia may be done by comparing the mutations with DNA from non-involved tissue or bodily fluid from the subject, or by comparing the mutations with mitochondrial DNA from a maternal relative. The progression of neoplasia may comprise monitoring the person at successive time periods for an increase in mutations or an increase in mutated mitochondrial genomes.

Another aspect of the invention, is a method of determining whether pre-neoplasia, neoplasia, or malignancy is latent or aggressive in its growth pattern, in a biological sample, comprising (a) providing a biological sample from the subject, (b) extracting DNA from the biological sample, (c) detecting the presence of mutations in the mtDNA, (d) determining whether the mutations are associated with normal interpopulation or intrapopulation variations, or whether the mutations are associated with pre-neoplasia, neoplasia or progression of neoplasia toward potential malignancy, and (e) repeating steps (a) through (d). The step of determining whether the mutations are associated with pre-neoplasia, neoplasia or progression of neoplasia may be done by comparing the mutations with DNA from non-involved tissue or bodily fluid from the subject, or by comparing the mutations with mitochondrial DNA from a maternal relative. The determination of whether the malignancy is latent or aggressive may comprise monitoring the person at successive time periods for an increase in mutations or an increase in mutated mitochondrial genomes.

Although specific mutation sites may indicate a disease state, a disorder or aging, the total mutation load is also important in determining the genesis, presence and progression of a disease, a disorder or aging. Accordingly, mutation load can be used to diagnose a disease, a disorder or aging.

The biological sample for the methods of the present invention may be taken from a tissue that is chosen from benign tissue, normal tissue, atypical tissue and histologically/pathologically abnormal tissue. Other clinical methods can also identify abnormal tissue. In addition, the sample may be taken from any bodily fluid, for example, blood, urine, prostate massage fluid, etc.

The step of determining whether the mutations are associated with normal interpopulation or intrapopulation variations, or whether the mutations are associated with pre-neoplasia, neoplasia, progression of neoplasia toward potential malignancy, or malignancy comprises comparing the mtDNA of the biological sample to a database of sequences associated with pre-neoplasia, neoplasia, progression of neoplasia toward potential malignancy, malignancy, inter and intra population variations and normal sequences. Optionally, one can determine total mutation load of the biological sample. Optionally, the step of determining whether the mutations are associated with pre-neoplasia, neoplasia or progression of neoplasia may be done by comparing the mutations mitochondrial DNA from non-involved tissue or bodily fluid from the subject, or by comparing the mutations with mitochondrial DNA from a maternal relative. The entire mitochondrial genome, or a subset of the genome, can be monitored for mutations which are then compared to the database to aid in the detection of pre-neoplasia, and/or neoplasia, progression towards malignancy and malignancy.

Another aspect of the invention is to provide oligonucleotide primers chosen from SEQ ID NO. 19 to 101. In still another embodiment of the invention, an oligonucleotide primer is provided which comprises a sequence of 10, 12, 14, 16, 18, 20, 22, 24, 26, or 30 contiguous nucleotides comprised of sequences from human mtDNA.

The methods, arrays and kits may comprise the mutations listed in Table 4 or SEQ ID Nos: 102 to 138.

Another aspect of the invention is provide a use of a primer to amplify a nucleic acid molecule comprising at least one mutation listed in Table 4 or SEQ ID Nos: 102 to 138. The primer may be selected from SEQ ID Nos: 19 to 101.

Another aspect of the invention is to provide a method of detecting at least one mutation listed in Table 4 or SEQ ID Ns: 102 to 138 in a nucleic acid molecule, comprising: amplifying the nucleic acid molecule with a primer associated with the mutation; and detecting the mutation. The primer may be selected from SEQ ID Nos: 19 to 101.

Another aspect of the present invention is to provide a use of a synonymous or non-synonymous mutation in an ND1 nucleic acid molecule of mtDNA, to detect a predisposition to prostate cancer, early detection of prostate cancer, genesis of prostate cancer, presence of prostate cancer, or progression of prostate cancer in a subject having mtDNA.

Another aspect of the present invention is to provide a use of a synonymous or non-synonymous mutation in an ND2 nucleic acid molecule of mtDNA, to detect a predisposition to prostate cancer, early detection of prostate cancer, genesis of prostate cancer, presence of prostate cancer, or progression of prostate cancer in a subject having mtDNA.

Another aspect of the present invention is to provide a use of a synonymous or non-synonymous mutation in a COX1 nucleic acid molecule of mtDNA, to detect a predisposition to prostate cancer, early detection of prostate cancer, genesis of prostate cancer, presence of prostate cancer, or progression of prostate cancer in a subject having mtDNA.

Another aspect of the present invention is to provide a use of a synonymous or non-synonymous mutation in a CytB nucleic acid molecule of mtDNA, to detect a predisposition to prostate cancer, early detection of prostate cancer, genesis of prostate cancer, presence of prostate cancer, or progression of prostate cancer in a subject having mtDNA.

The use of the synonymous or non-synonymous mutation of the above can be detected in malignant tissue, adjacent benign tissue, or distant benign tissue in a subject having prostate cancer or progressing toward prostate cancer.

Another aspect of the invention is to provide a method of detecting a mutation associated with prostate cancer in a subject having mtDNA, comprising: (a) providing a biological sample from the subject, wherein the biological sample is chosen from non-involved tissue, distant benign tissue, adjacent benign tissue, atypical tissue, histologically abnormal tissue, and malignant tissue; (b) extracting DNA from the biological sample; (c) detecting the presence of the mutation in ND1, ND2, COX1 or CytB of mtDNA; and (d) determining whether the mutation is associated with normal interpopulation or intrapopulation variations, or whether the mutation is associated with prostate cancer. The method may further comprise at least one of (i) determining total mutation load in the mtDNA of the biological sample; and (ii) determining the identity of the mutation in the mtDNA of the biological sample. The step of determining whether the mutation is associated with normal interpopulation or intrapopulation variations, or whether the mutation is associated with prostate cancer, may comprises at least one of: (A) comparing the mtDNA of the biological sample to a database, the database containing data of interpopulation and intrapopulation variations, and mutations associated with prostate cancer; and (B) determining total mutation load of the biological sample. The method may be used in a diagnosis, wherein the diagnosis is chosen from predisposition to prostate cancer, early detection of prostate cancer, genesis of prostate cancer, presence of prostate cancer, and progression of prostate cancer.

The step of detecting the presence of mutations may be chosen from sequencing the mtDNA, amplifying mtDNA by PCR, Southern, Northern, Western and South-Western blot hybridizations, denaturing HPLC, hybridization to microarrays, gene chips or biochips, molecular marker analysis, and a combination of any of the above. The mitochondrial DNA which is sequenced may comprise the entire mitochondrial genome. The mutation may be a synonymous or non-synonymous mutation. Finally, the mutation may be heteroplasmic at any level.

Another aspect of the invention is to provide an array comprising a plurality of nucleic acid members, and a solid substrate, wherein each of the nucleic acid members is associated with at least one mutation in ND1, ND2, COX1 or CytB of the mitochondrial genome and is indicative of the presence of prostate cancer, and is chosen from mitochondrial DNA, RNA transcribed from mitochondrial DNA, and cDNA, wherein each nucleic acid member has a unique position on said array and is stably associated with the solid substrate.

Another aspect of the invention is to provide an array comprising a plurality of nucleic acid members, and a solid substrate, wherein each of the nucleic acid members is associated with at least one mutation in ND1, ND2, COX1 or CytB of the mitochondrial genome and is indicative of the progression toward prostate cancer, and is chosen from mitochondrial DNA, RNA transcribed from mitochondrial DNA, and CDNA, wherein each nucleic acid member has a unique position on said array and is stably associated with the solid substrate.

Another aspect of the invention is to provide a kit for diagnosing prostate cancer comprising a disposable chip, the array described above, means for holding the disposable chip, means for extraction of mitochondrial DNA and means for access to a database of mitochondrial DNA sequences.

Another aspect of the invention is to provide a kit for determining the progression toward prostate cancer or early detection of prostate cancer comprising a disposable chip, the array described above, means for holding the disposable chip, means for extraction of mitochondrial DNA and means for access to a database of mitochondrial DNA sequences.

Another aspect of the invention is to provide a database containing mitochondrial DNA sequences for ND1, ND2, COX1 and CytB, the sequences are chosen from normal control sequences associated with non-disease states, sequences associated with interpopulation variations, sequences associated with intrapopulation variations, and sequences associated with prostate cancer.

Another aspect of the invention is to provide a method of monitoring a person for the progression toward prostate cancer or progression of prostate cancer, in a biological sample from a subject, comprising, (a) providing a biological sample from the subject; (b) extracting DNA from the biological sample; (c) detecting the presence of mutations in the ND1, ND2, COX1 or CytB genes of the mtDNA; (d) determining whether the mutations are associated with normal interpopulation or intrapopulation variations, or whether the mutations are associated with presence of a predisposition to prostate cancer, progression toward prostate cancer, prostate cancer or progression of prostate cancer, and; (e) repeating steps (a) to (d). The biological sample may be from a tissue that is chosen from normal tissue, distant benign tissue, adjacent benign tissue, diseased tissue and malignant tissue. The step of detecting the presence of mutations in the ND1, ND2, COX1 and CytB genes of mitochondrial DNA may comprise at least one of: (a) comparing the mtDNA of the biological sample to the database described above; (b) comparing the mtDNA of the biological sample to a sample of mtDNA from non-involved tissue or non-involved bodily fluid from the subject; (c) comparing the mtDNA of the biological sample to a sample of mtDNA from a maternal relative of the subject; (d) determining total mutation load of the biological sample; and (e) identifying the mutations. The mutations may be synonymous or non-synonymous. The methods may further comprise monitoring the subject at successive time periods for an increase in mutations in ND1, ND2, COX1 or CytB, or an increase in the number of mitochondrial genomes having mutations in ND1, ND2, COX1 or CytB.

Another aspect of the invention is to provide an isolated mitochondrial ND1 nucleic acid molecule comprising at least one synonymous or non-synonymous mutation for use in detecting the progression toward prostate cancer or the presence of prostate cancer in a subject.

Another aspect of the invention is to provide an isolated mitochondrial ND2 nucleic acid molecule comprising at least one synonymous or non-synonymous mutation for use in detecting the progression toward prostate cancer or the presence of prostate cancer in a subject.

Another aspect of the invention is to provide an isolated mitochondrial COX1 nucleic acid molecule comprising at least one synonymous or non-synonymous mutation for use in detecting the progression toward prostate cancer or the presence of prostate cancer in a subject.

An aspect of the invention is to provide an isolated mitochondrial CytB nucleic acid molecule comprising at least one synonymous or non-synonymous mutation for use in detecting the progression toward prostate cancer or the presence of prostate cancer in a subject.

### BRIEF DESCRIPTION OF THE FIGURES

FIG. 1 is a histogram showing the number of mutations at nucleotide position in mitochondrial DNA from patients with prostate cancer.

FIG. 2 shows the first half of the non-synonymous clusters produced using Hierarchal Clustering Explorer (HCE).

FIG. 3 shows the second half of the non-synonymous clusters produced using Hierarchal Clustering Explorer (HCE).

FIG. 4 is a copy of FIG. 3 wherein the important cluster is shaded.

### BRIEF DESCRIPTION OF THE TABLES

Table 1 is a summary of mutations associated with aging.

Table 1a is a principal component analysis of mutations in mtDNA of seven protein coding regions in control, distant benign, adjacent benign and malignant tissue.

Table 1b is a neural network analysis of mutations in mtDNA of seven protein coding regions in control, distant benign, adjacent benign and malignant tissue.

Table 1c is a summary of the synonymous and non-synonymous mutations found in ND1, ND2, COXI and CYTB regions of the mitochondria of 31 patients having prostate cancer from distant benign, adjacent benign and malignant prostate tissue.

Table 1d is a Chi square analysis of mutations in mitochondrial DNA in distant benign tissue from malignant glands versus prostate tissue from symptomatic but not malignant subjects.

Table 2 is a summary of the mean number of deletions is epidermal tumours and adjacent normal tissues.

Table 3 is summary of the standard method of DHPLC.

Table 4 is a summary of mitochondrial mutations (including D-loop) from prostate needle biopsies and complete genome mutations from malignant, adjacent and distant benign prostate glands from patients with prostate cancer.

Table 5 is a list of primers used for complete mitochondrial genome amplification for formalin fixed and normal tissues from blood.

### DETAILED DESCRIPTION OF THE INVENTION

The method of the present invention can be used to diagnose diseases linked to mtDNA. The method of the present invention provides for analysis of the mitochondrial genome of an individual from a biological sample, for example by amplification of the mitochondrial genome, sequencing a portion of the mitochondrial genome, preferably the entire mitochondrial genome of the individual using any known means. Denaturing high performance liquid chromatography (DHPLC) may also be used to rapidly screen many samples. DHPLC can focus on hotspots of mutations. DHPLC is more sensitive than automated sequencing in terms of detecting mutations, and can even detect 2% heteroplasmy, compared with 20-25% for ordinary sequencing. Methods for detecting lower levels of heteroplasmy (<2%) may also be developed.

As used herein, the “presence” of a mutation in mtDNA includes heteroplasmic mutations and, therefore, it is contemplated that there may be additionally the presence of some normal mtDNA in a sample in which the mutated DNA is present.

As used herein, “actinic kerotoses” means proposed precursor epidermal lesion of a squamous cell carcinoma.

As used herein, “aging” refers to an accumulation of changes with time, both at the molecular and cellular levels.

As used herein, “alleles” means one of several alternative forms of a given DNA sequence occupying a specific place on a chromosome.

As used herein, “attaching” or “spotting” refers to a process of depositing a nucleic acid onto a solid substrate to form a nucleic acid array such that the nucleic acid is irreversibly bound to the solid substrate via covalent bonds, hydrogen bonds or ionic interactions.

As used herein, “atypical” or “abnormal” means cellular appearance which is not normal, but also does not appear to be malignant.

As used herein, “basal cell carcinoma” means a type of cancer of skin cells.

As used herein, “benign” means of no danger to health; not recurrent or progressive; not malignant.

As used herein, “Bowen's disease” means in situ epidermal carcinoma.

As used herein, “diagnostic” or “diagnosing” means using the presence or absence of a mutation or combination of mutations as a factor in disease diagnosis or management. The detection of the mutation(s) can be a step in the disease state diagnosis.

As used herein, “disease” includes a disorder or other abnormal physical state.

As used herein, “disease associated mitochondiral genomes” means genomes containing mutations indicative or otherwise associated with a particular disease.

As used herein, “database” means an electronic storage system (computer based using standard industry software) which will have the capacity to store and provide retrievable information that will enable researchers to rapidly determine the structure of the nucleotide sequences. The database will also store descriptive information about those individuals who provide the biological samples. This descriptive information will include health status and other pertinent indices which may be correlated to the biological sample.

As used herein, “deletions” means removal of a region of DNA from a contiguous sequence of nucleic acids, where once a deletion has occurred, the gap is repaired by rejoining of the ends. Deletions can range in size from one base to thousands of bases or larger.

As used herein, “duplications” means when a specific sequence of DNA is copied and inserted behind or forward of the original copy one or more times or elsewhere in the genome.

As used herein, “heteroplasmy” is defined by the ratio of mutations in the mitochondrial sequences within one organ or cell. Heteroplasmic mutations are those mutations which occur in some, but not all of the copies of the mitochondrial genome.

As used herein, “homoplasmy” means all mitochondrial sequences are identical.

As used herein, “hyper-mutation” means accelerated mutation rate which cannot be explained by normal cellular processes or standard evolutionary principles.

As used herein, “inversions” refers to when a length of DNA is excised and reinserted in reverse orientation.

As used herein, “maternal inheritance” means mitochondria which are inherited through the cytoplasm of the ovum.

As used herein, “maternal line” refers to the clonal sequence of mitochondrial DNA as passed down through successive generations from the mother.

As used herein, “mitochondria” means a eukaryotic cytoplasmic organelle that generates ATP for cellular processes.

As used herein, “mutation” encompasses any change in a DNA sequence from the wild type sequence, including without limitation point mutations, transitions, insertions, transversions, translocations, deletions, inversions, duplications, recombinations or combinations thereof.

As used herein, “mutation load” refers to an increase in mutations in mtDNA which may eventually lead to compromised function of the involved gene or the entire genome or may accumulate in non-coding regions.

As used herein, “neoplasia” means a pathological process which may result in transformation to malignant status.

As used herein, “non-involved tissue” means tissue from a part of the body which is not associated with the disease in question.

As used herein, a “non-synonymous” mutation is a mutation of a polynucleotide which results in a different encoded amino acid.

As used herein, “normal tissue” means tissue with no visible manifestations of disease as determined by histology.

As defined herein, a “nucleic acid array” refers to a plurality of unique nucleic acids attached to one surface of a solid support at a density exceeding 20 different nucleic acids/cm² wherein each of the nucleic acids is attached to the surface of the solid support in a non-identical preselected region. In one embodiment, the nucleic acid attached to the surface of the solid support is DNA. In a preferred embodiment, the nucleic acid attached to the surface of the solid support is cDNA. In another preferred embodiment, the nucleic acid attached to the surface of the solid support is cDNA synthesized by polymerase chain reaction (PCR). Preferably, a nucleic acid array according to the invention, comprises nucleic acids of at least 150 nucleotides in length. Preferably, a nucleic acid array comprises nucleic acids of less than 6,000 nucleotides in length. More preferably, a nucleic acid array comprises nucleic acids of less than 500 nucleotides in length. In one embodiment, the array comprises at least 500 different nucleic acids attached to one surface of the solid support. In another embodiment, the array comprises at least 10 different nucleic acids attached to one surface of the solid support. In yet another embodiment, the array comprises at least 10,000 different nucleic acids attached to one surface of the solid support. The term “nucleic acid”, as used herein, is interchangeable with the term “polynucleotide”.

As used herein, a “nucleic acid target” or “a target nucleic acid” is defined as a nucleic acid capable of binding to a nucleic acid member of complementary sequence through one or more types of chemical bonds, usually through complementary base pairing, usually through hydrogen bond formation. As used herein, a nucleic acid target may include natural (i. e., A, G, C, or T) or modified bases (7-deazaguanosine, inosine, etc.). In addition, the bases in nucleic acid probe may be joined by a linkage other than a phosphodiester bond, so long as it does not interfere with hybridization. Thus, nucleic acid targets may be peptide nucleic acids in which the constituent bases are joined by peptide bonds rather than phosphodiester linkages. Preferably, the nucleic acid targets are derived from human tissue or fluid extracts. More preferably, the nucleic acid targets are single- or double-stranded DNA, RNA, or DNA-RNA hybrids synthesized from human tissue of fluid extracts.

As used herein, “nucleus” means the most conspicuous organelle in the eucaryotic cell, contains all of the chromasomal DNA.

As used herein, “PSA Test” means prostate-specific antigen test; an antigen found in blood that may be indicative of cancer of the prostate.

As used herein, “point mutation” means the change of a single nucleotide in DNA.

As used herein, “polymorphism” means sequence variation in a population of alleles or mtDNA genomes.

As used herein, “precursor lesions” means a DNA mutation, or combinations thereof, indicating potential disease association.

As used herein, “predisposed to a disease” or a “predisposition to a disease” means that individuals are at higher risk for developing the disease or disorder or are at higher risk for early onset of the disease or disorder than the average individual, due to the presence or absence of mutations which are associated with the disease or disorder.

As used herein, “pre-neoplasia” means indications at the cellular or DNA level that a cell may be on the threshold of becoming neoplastic.

As used herein, “preselected region”, “predefined region”, or “unique position” refers to a localized area on a substrate which is, was, or is intended to be used for the deposit of a nucleic acid and is otherwise referred to herein in the alternative as a “selected region” or simply a “region.” The preselected region may have any convenient shape, e.g., circular, rectangular, elliptical, wedge-shaped, etc. In some embodiments, a preselected region is smaller than about 1 cm², more preferably less than 1 mm², still more preferably less than 0.5 mm², and in some embodiments about 0.125 to 0.5 mm².

As used herein, “somatic mutation” means a change in DNA sequence after fertilization.

As used herein, “solid substrate” or “solid support” refers to a material having a rigid or semi-rigid surface. The terms “substrate” and “support” are used interchangeable herein with the terms “solid substrate” and “solid support”. The solid support may be biological, non-biological, organic, inorganic, or a combination of any of these, existing as particles, strands, precipitates, gels, sheets, tubing, spheres, containers, capillaries, pads, slices, films, plates, slides, etc. Often, the substrate is a silicon or glass surface, (poly)tetrafluoroethylene, (poly)vinylidendifluoride, polystyrene, polycarbonate, a charged membrane, such as nylon 66 or nitrocellulose, or combinations thereof. In a preferred embodiment, the solid support is glass. Preferably, at least one surface of the substrate will be substantially flat. Preferably, the surface of the solid support will contain reactive groups, including, but not limited to, carboxyl, amino, hydroxyl, thiol, or the like. In one embodiment, the surface is optically transparent.

As used herein, “squamous cell carcinoma” means a type of cancer of skin cells.

As used herein, “stably associated” refers to a nucleic acid that is irreversibly bound to a solid substrate to form an array via covalent bonds, hydrogen bonds or ionic interactions such that the nucleic acid retains its unique preselected position relative to all other nucleic acids that are stably associated with an array, or to all other preselected regions on the solid substrate under conditions wherein an array is analyzed (i.e., hybridization and scanning).

A “statistically significant” number of mitochondrial DNA sequences is determined by or through the use of standard chi-square statistical algorithms using or determining observed versus expected scores.

As used herein, “subtle mutation” means low level of mutation at the threshold of detection.

As used herein, a “synonymous” mutation is a mutation in a polynucleotide which does not have an affect on the encoded amino acid.

As used herein, “transitions” means substitution of like nitrogenous bases, pyrimidine to pyrimidine, purine to purine. A mutation in which one pyrimidine is substituted by the other, or in which one purine is substituted by the other.

As used herein, “transversions” means substitution of unlike nitrogenous bases, purine to pyrimidine, pyrimidine to purine. A mutation in which a purine is substituted or replaced by a pyrimidine or vice versa.

MtDNA and Diagnosis of Specific Diseases

In an embodiment of the present invention, methods are provided for monitoring aging and diagnosing specific diseases such as prostate cancer and non-melanoma skin cancer through comparisons of mtDNA sequences. Diagnosing diseases such as prostate cancer with mtDNA, rather than nuclear DNA has several advantages. Firstly, mtDNA, a less complex genome, is easily understood at an individual and population level, hence a large mtDNA database with normal and disease associated genomes renders individual diagnosis extremely accurate. Accordingly, variation, in relationship to disease, is understood. Secondly, mtDNA has a 10-fold higher mutation rate than nuclear DNA (Wallace 1992). Nuclear rearrangements, suggestive of preliminary disease, are rapidly communicated to mitochondria, where they appear as somatic mutations. Thirdly, mtDNA has a maternal inheritance pattern, and is essentially clonal in that all mitochondria begin with the same mtDNA sequence, hence variation from this clonal condition is easily detected. Additionally, mtDNA does not show convincing evidence of recombination, thus any alterations in sequence are a somatic event. Any one mitochondrion harboring a mutation(s) is in a sense ‘recessive’ as a consequence of there being many mitochondrial genomes (2-10 copies) per mitochondrion, and many mitochondria per cell (500-2,000). Moreover, mitochondrial genomes can tolerate very high levels (up to 90%) of mitochondria with damaged genomes. This happens through complementation by the remaining wild type mtDNA (Chomyn et al. 1992). However, mutated genomes have a replicative advantage over wild type genomes because they are usually smaller (Hayashi et al. 1991), hence there is clonal expansion of mutated mtDNA (Brierley et al. 1998), suggesting that unlike nuclear genes, there is little or no selection against cells harboring mtDNA mutations. Because of this elevated mutation rate, mutations and/or deletions that appear in mtDNA are maintained through the life span of the cell and may serve as a record of exposures to various mutagens. The integrity of mtDNA is maintained by nuclear repair mechanisms, and a defect at these loci has been suggested to result in an autosomal dominant disorder associated with multiple mitochondrial deletions (Zeviani et al. 1990). Consequently, mtDNA may function as an early warning sentinel of early nuclear events related to a variety of cancers or other diseases. Finally, the mitochondrial genome can be sequenced and monitored for mutations on an individual basis.

The methods and products of the present invention detect both heteroplasmic as well as homoplasmic mutations. In fact, heteroplasmic mutations may be key to the detection of the early genesis of disease, disorder or aging. In addition, although specific mutation sites may indicate a particular disease state, disorder or aging process, the total mutation load is also important in determining the genesis, presence and progression of a disease, a disorder or aging.

The present invention allows for the ability to examine benign or normal tissue or bodily fluids to determine the genesis of disease, disorder or aging. For example, the present invention allows for the ability to examine benign tissue or bodily fluids for the presence of pre-neoplasia, neoplasia, progression toward malignancy and malignancy.

The mitochondrial mutations detected by the methods of the invention are compared to inter and intrapopulation variations in mitochondrial DNA, and may include comparison with mitochondrial DNA from non-involved tissue from the subject, or with mitochondrial DNA from a maternal relative. It is not necessary to analyze the entire mitochondrial genome. For example, it is not necessary to sequence the entire mitochondrial genome, only a select portion of it. Accordingly, a sample of mitochondrial DNA can provide a diagnosis.

Diagnosis of Non-Melanoma Skin Cancer

In a preferred embodiment of the invention, a system for early diagnosis of mtDNA changes in non-melanoma skin cancer (NMSC) and their precursor lesions indicative of solid tumour development is provided. The particular changes, such as the common deletion and associated mutations, and the incidence of as yet uncharacterised deletions in mtDNA serve as reliable bio-markers of potential skin cancer. The mutation fingerprint of the entire mtDNA genome in human NMSC and its precursor lesions is determined. Thus mtDNA changes are established as an early bio-marker of human skin cancer and its precursor lesions. Denaturing HPLC can then be used to assess low levels of heteroplasmy at the sequences of interest. This approach can also provide an insight into the development of early changes in other human tumours.

Diagnosis of Prostate Cancer

In another embodiment of the invention, a system for diagnosis of prostate cancer is provided. Age related accumulation of mtDNA defects might predispose an individual to the appearance of certain clinical disorders such as prostate cancer which is prevalent in middle age and older men. In a preferred embodiment, routine prostate cancer screening takes place through mitochondrial genome sequencing from prostate massage fluid. The presence of epithelial cells transformed into cancer cells, can be determined through amplification of mtDNA from prostate massage fluid, eclipsing current diagnostic techniques such as digital rectal examination and PSA. Recently Fliss et al. (2000) identified mutated mtDNA in urine samples of patients with bladder cancer. Similar findings in prostate massage fluid provide a non-invasive early detection method for prostate cancer. Different types of prostate cancer can be diagnosed, as well as differentiating between aggressive, fast growing cells in younger patients in contrast to prostate cancer as a whole.

Early Detection and Monitoring of Prostate Cancer Progression

The system and method of the present invention may be used to detect cancer, and in particular prostate cancer, at an early stage, and before any histological abnormalities. For example, the system and method of the present invention may be used to detect pre-neoplasia in prostate tissue. The system can be used to detect the genesis and progression of prostate cancer. Mutations, including both subtle and hyper-mutation (Chen et al. 2002; Chen et al. 2003) in mitochondrial DNA from human prostate tissue, or fluid associated with the prostate (for example prostate massage fluid or urine), can be tested for the presence of neoplasia, and retested at intervals to follow cancer transformation, diagnose malignancy, or confirm continued benign status.

These mutations are determined by comparison to mitochondria extracted from non-involved tissue such as, but not limited to: blood, urine, hair and buccal swabs. This direct comparison eliminates polymorphisms, maternal background or normal haplotype variation unassociated with disease. The mutations can also be compared to mitochondrial sequences associated with inter and intrapopulation variations. One or more mutations from fluid or tissue of the organ or body system in question, indicates possible disease genesis. The person is then monitored, at successive intervals, for an increase in mutations at other sites, and/or an increase in the number of mutated mitochondrial genomes, indicating disease progression. Benign tissue from the prostate cannot always be considered non-involved. In fact, as can be seen in Example 9, below, what appears to be benign tissue may contain mitochondrial mutations associated with pre-neoplasia, neoplasia, progression toward malignancy or malignancy. In addition, mutation load rather than specific mutations may be instrumental in determining disease and progression of disease. The system and method of the present invention detects heteroplasmic as well as homoplasmic mutations.

The prostate gland is monitored for mutations in the mitochondrial genome through prostate massage fluid (PMF) taken during an initial digital rectal examination (DRE) of the prostate. Cells within the PMF are concentrated, smeared on a slide and stained with PSA immunoperoxidase for identification of prostate epithelial cells. These prostate cells are selectively recovered through laser capture micro-dissection. The mitochondrial DNA from these cells is analyzed and compared to mitochondrial DNA from non-involved tissue, and to sequences of inter and intrapopulation variations. For example, the DNA analysis can comprise sequencing of the mtDNA. Total DNA is extracted from these cells and mitochondrial specific primers, designed for use with biopsy material treated with formalin (Table 5), are used to amplify the entire mtDNA genome with overlapping amplicons. These PCR products are then sequenced by methods well known to those in the art, including DNA resequencing arrays. Sequencing results are screened for heteroplasmies and mutations and compared to a database of known mtDNA mutations associated with malignant and benign prostate tissues. Based on these comparisons a designation is returned as to the condition of the prostate in regards to, but not limited to: benign (no mutations); pre-neoplasia or neoplasia (low level of mutations); or malignancy (high level of mutations). In the situation of benign, pre-neoplasia and neoplasia, the prostate can be monitored for progression through regular PMF screenings as described.

Alternatively, biopsy material which has been diagnosed as benign, atypical, abnormal can undergo similar testing by either laser capture micro-dissection of the biopsy, or the tissue can be scrapped off the slides, followed by DNA extraction, amplification, sequencing and database comparison.

As an alternative to sequencing, and comparison to a database, micro-array technology could be used to identify a specific pattern of mutations, or mutation load based on any number, or combination of the mutations listed in Table 4, through the construction of oligonucleotides, or a specific set of oligonucleotides.

Disease progression can be monitored by comparing mtDNA mutations at successive intervals to a database of mutations in mitochondrial genomes associated with pre-neoplasia, neoplasia and prostate cancer, including calculation of total mutation load. Prostate biopsy tissue can be tested for pre-neoplasia, neoplasia and/or malignant progression in cells described clinically as benign, normal, atypical or abnormal by common histological/pathological, or other clinical methods.

Assessment of Mutations Associated with Aging

The system and method of the present invention may be used to assess aging, based on the increasing frequency of mutations such as the “common deletion” of 4977-bp and other mutations of the mitochondrial genome (Liu et al. 1997). This information, in conjunction with health survey data, allows crucial statistical discrimination between separate causes resulting in the same mutation/deletion. Fortunately mtDNA is inherited exclusively through the ovum and is essentially clonal in nature (Van De Graaff & Fox, 1995). This permits carefully controlled studies of mutations/deletions within maternal lines through several generations to determine a reliable age related deletion frequency. This information may be used to develop treatment methods which slow the aging process.

Collection of Samples

Biological samples can be collected by any known means, whether for the purpose of constructing a mtDNA sequence database, or performing a diagnostic test on an individual. Samples destined for database generation include, but are not limited to: tumour banks, maternal lineage studies involving affected and unaffected individuals from the same maternal lineage, as well as maternal lineage studies from groups or populations with high frequencies of specific disease such as, but not limited to: skin and prostate cancer, assessment of health status and aging. For example, FTA® Gene Cards® may be used to collect and archive biological samples. Suitable samples include any tissue or body fluid derived from mesothelium, epithelium, or endothelium. Such tissues and fluids include, but are not limited to blood, sputum, buccal cells, saliva, prostate massage fluid, sweat, bone, hair, lymph tissue, cervical smears, breast aspirate, fecal matter, ejaculate, menstrual flow and biopsy tissue. Preferably, approximately 100 μl of blood, 100 μg to 25 mg of solid tissue is sampled. In the case of suspected skin cancer, skin cells or tissue, (from normal, NMSC and precursor lesions) is taken from skin biopsy or a routine suction blistering technique. Where a disease is suspected, primary care physicians, oncologists or other practitioners, may extract both normal and suspected disease tissue from the patient.

For samples of tumours such as prostate or skin, replicate cross-sections (5 microns) of micro-dissected paraffin embedded tissues are de-paraffinized prior to one slide being stained with hematoxylin and eosin (HE), with the replicate stained with methyl green (MG), as is standard in the art. HE stains are graded by a pathologist for normal, precursor, and applicable grades of tumour progression. Replicate MG slides are used for laser capture, according to manufacturers recommendations (Arcturus) of graded cells.

Extraction of mtDNA

Extraction of DNA may take place using any method known in the art, followed by sequencing of the mitochondrial genome, as described in Current Protocols in Molecular Biology.

Analyzing mtDNA

The step of detecting the presence of mutations in the mtDNA can be selected from any technique as is known to those skilled in the art. For example, analyzing mtDNA can comprise sequencing the mtDNA, amplifying mtDNA by PCR, Southern, Northern, Western South-Western blot hybridizations, denaturing HPLC, hybridization to microarrays, biochips or gene chips, molecular marker analysis, biosensors, melting temperature profiling or a combination of any of the above. In addition, statistical techniques such as Inductive Rule Extraction, Neural Networking and Wave Analysis can be used.

Sequencing of MtDNA

PCR

Polynucleotide sequences of the invention can be amplified by the polymerase chain reaction (PCR). PCR methods are well-known to those skilled in the art. PCR requires the presence of a nucleic acid to be amplified, two single stranded oligonucleotide primers flanking the sequence to be amplified, a DNA polymerase, deoxyribonucleoside triphosphates, a buffer and salts. The method of PCR is well known in the art. PCR is performed as described in Mullis and Faloona, 1987, Methods Enzymol., 155: 335, herein incorporated by reference.

In general, PCR is performed using template DNA (at least 1 fg; more usefully, 1-1000 ng) and at least 25 pmol of oligonucleotide primers. A typical reaction mixture includes: 2 μl of DNA, 25 pmol of oligonucleotide primer, 2.5 μl of 10×PCR buffer 1 (Perkin-Elmer, Foster City, Calif.), 0.4 μl of 1.25 μM dNTP, 0.15 μl (or 2.5 units) of Taq DNA polymerase (Perkin Elmer, Foster City, Calif.) and deionized water to a total volume of 25 μl. Mineral oil is overlaid and the PCR is performed using a programmable thermal cycler.

The length and temperature of each step of a PCR cycle, as well as the number of cycles, are adjusted according to the stringency requirements in effect. Annealing temperature and timing are determined both by the efficiency with which a primer is expected to anneal to a template and the degree of mismatch that is to be tolerated. The ability to optimize the stringency of primer annealing conditions is well within the knowledge of one of moderate skill in the art. An annealing temperature of between 30° C. and 72° C. is used. In general, initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above; 1-2 minutes), and extension (72° C. for 1 minute). The final extension step is generally carried out for 4 minutes at 72° C., and may be followed by an indefinite (0-24 hour) step at 4° C.

DNA Sequencing

Any known means to sequence the mitochondrial genome may be used. Preferably, mtDNA is amplified by PCR prior to sequencing. PCR products can be sequenced directly or cloned into a vector which is then placed into a bacterial host. Examples of DNA sequencing methods are found in Brumley, R. L. Jr. and Smith, L. M., 1991, Rapid DNA sequencing by horizontal ultrathin gel electrophoresis, Nucleic Acids Res. 19:4121-4126 and Luckey, J. A., et al, 1993, High speed DNA sequencing by capillary gel electrophoresis, Methods Enzymol. 218: 154-172. The combined use of PCR and sequencing of mtDNA is described in Hopgood, R., et al, 1992, Strategies for automated sequencing of human mtDNA directly from PCR products, Biotechniques 13:82-92 and Tanaka, M. et al, 1996, Automated sequencing of mtDNA, Methods Enzymol. 264: 407-421

Deletion Analysis and Detection

A preferable approach is the long extension PCR (LX-PCR) technique using the Expand Long Template PCR system (Boehringer Mannheim). Using the LX-PCR technique, which has been established and validated in the Birch-Machin laboratory (Ray et al. 2000), there is the opportunity to rapidly screen for the whole spectrum of mtDNA deletions as opposed to the incidence of a single deletion.

A semi-quantitative PCR method (Corral-Debrinski et al 1991) can be used to estimate the proportion of the mtDNA⁴⁹⁷⁷ deletion in the total mtDNA.

In addition, Southern Blot and probing technology labeled with isotopes or any other technique as is standard in the art may be used for deletion detection as well.

Sequencing of PCR Products

Any known means may be used to sequence the PCR products. Preferably, the entire DNA sequence is characterized by di-deoxy sequencing using ABI Big Dye Terminator™ technology and a series of 72 overlapping primers each for heavy and light strands. Sequencing occurs on one, several, or a combination of ABI platforms such as the 310, 3100, or 3700. Sequencing reactions are performed according to manufacturer's recommendation.

Mutational Analysis of the Mitochondrial Genome Using Denaturing High Performance Liquid Chromatography (DHPLC)

Prior to sequencing of the mitochrondrial genome and identification of mutational hotspots, DHPLC can be used to rapidly screen mutations in many samples. This technique provides greater sensitivity in identification of low levels of heteroplasmy. It cannot detect homoplasmic changes but will complement traditional sequencing. Apart from the homoplasmic mutations recently identified in tumours, the vast majority of reported mtDNA mutations are heteroplasmic (Chinnery et al. 1999). These heteroplasmic mtDNA changes result in the formation of heteroduplexes after PCR amplification of the mtDNA. Rapid screening for heteroplasmic mtDNA mutations is determined using the relatively new technique of denaturing high performance liquid chromatography (DHPLC) (Oefner & Underhill, 1998). This technique has recently been used to rapidly screen and identify whole mtDNA genomes for heteroplasmic point mutations down to levels <5% (Van den Bosch et al. 2000).

The DHPLC may be performed on the WAVE™ DNA Fragment Analysis System (Transgenomic, Omaha, USA) which provides a fully automated screening procedure. The same technology can be used to screen for mtDNA heteroplasmic mutations. Preferably, the entire mtDNA genome is amplified by PCR in 13 overlapping fragments using two different PCR conditions as described by van den Bosch et al. (2000). The 1-2 kb PCR products are digested into fragments of 90-600 bp and resolved at their optimal melting temperature. Mutations are represented as two peaks and mutations with low percentages, such as <2% heteroplasmy as a ‘shoulder’ in the peak.

DNA sequencing can also take place using a microarray, as is known in the art (Chee et al. 1996).

Data Analysis

Once sequenced, normal and disease associated mtDNA sequences are archived for comparison in a database. Resequencing devices, micro-array technology, integrated microfluidic amplification and analysis systems, high-speed, high-throughput, mutation detection, and other methods may all be used with the methods of the present invention.

Data obtained from the sequencing of the individual mitochondrial genome is compared to population level data. The data is obtained through obtaining samples and sequencing mtDNA as described above. Preferably, the database contains information from maternal line studies. The population level data is maintained in a database. Any suitable database can be used.

Preferably, a multidimensional evaluation research database of clinical and biological data is used, which provides the bio-informatics infrastructure necessary for the collection, processing and dissemination of information amassed by the laboratories involved in this venture. The database is a centralized electronic system which links networks resulting in a dynamic and powerful resource.

The database may be accessed through any known means, and preferably through a secure Internet pathway. Preferably, the database is developed using an e-commerce algorithm, built on a server and deployed using an application server which supports a high volume of concurrent users through optimized performance and scalability features. A separate “web” server can provide the foundation of the web-site architecture since it can serve as the central point through which all content, applications, and transactions must flow before reaching users.

Data mining algorithms known in the art are used to discover patterns, clusters and models from data (SAS 2000). Moreover, intelligent algorithms and methods will be developed for: occurrence of mutation and mutation rates, patterns of mutations for disease detection, information retrieval, and other complex sequence analysis software.

Nucleic Acid Members and Probes

The invention provides for nucleic acid members and probes that bind specifically to a target nucleic acid sequence. The target nucleic acid sequence is a nucleic acid or a region of a nucleic acid that is to be detected, as indicative of disease such as prostate cancer, non-melanoma skin cancer and the like. The target nucleic acid sequences to be analyzed using a microarray of the invention are preferably derived from human tissue or fluid samples. The invention provides for target nucleic acid sequences comprising RNA or nucleic acid corresponding to RNA, (i.e., cDNA), or DNA. Nucleic acid members are stably associated with a solid support to comprise an array according to the invention. The nucleic acid members may be single or double stranded, and may be a PCR fragment amplified from cDNA.

The invention also provides for polynucleotide sequences comprising a probe. As used herein, the term “probe” refers to an oligonucleotide which forms a duplex structure with a sequence in the target nucleic acid, due to complementarity of at least one sequence in the probe with a sequence in the target region. The probe may be labeled, according to methods known in the art. A probe according to the invention may be single or double stranded.

Diagnostic Devices

The invention includes diagnostic devices such as biochips, gene chips or microarrays used to diagnose specific diseases or identify specific mutations. All sequenced mitochondrial genomes are assessed to create a consenus structure of the base pair arrangement and are assigned a prohibiting index for proportion of base pair deletions and mutations associated with a particular disease or disorder. The diagnostic arrangement is then used to create biochips, gene chips, or microarrays.

Once sequences associated with particular diseases, disease states or disorders are identified, hybridization of mtDNA to an array of oligonucleotides can be used to identify particular mutations. Any known method of hybridization may be used. Preferably, an array is used, which has oligonucleotide probes matching the wild type or mutated region, and a control probe. Commercially available arrays such as microarrays or gene chips are suitable. These arrays contain thousands of matched and control pairs of probes on a slide or microchip, and are capable of sequencing the entire genome very quickly. Review articles describing the use of microarrays in genome and DNA sequence analysis is available at www.gene-chips.com.

Microarray

Polynucleotide arrays provide a high throughput technique that can assay a large number of polynucleotides in a sample comprising one or more target nucleic acid sequences. The arrays of the invention are useful for gene expression analysis, diagnosis of disease and prognosis of disease (e.g., monitoring a patient's response to therapy, drug screening, and the like).

Any combination of the polynucleotide sequences of mtDNA indicative of disease, aging, or other health related mutations are used for the construction of a microarray.

The target nucleic acid samples to be analyzed using a microarray are derived from any human tissue or fluid which contains adequate amounts of mtDNA, as previously described, preferably prostate massage fluid, solid tumours, blood, or urine. The target nucleic acid samples are contacted with polynucleotide members under hybridization conditions sufficient to produce a hybridization pattern of complementary nucleic acid members/target complexes.

Construction of a Microarray

The microarray comprises a plurality of unique polynucleotides attached to one surface of a solid support, wherein each of the polynucleotides is attached to the surface of the solid support in a non-identical preselected region. Each associated sample on the array comprises a polynucleotide composition, of known identity, usually of known sequence, as described in greater detail below. Any conceivable substrate may be employed in the invention.

The array is constructed using any known means. The nucleic acid members may be produced using established techniques such as polymerase chain reaction (PCR) and reverse transcription (RT). These methods are similar to those currently known in the art (see e.g. PCR Strategies, Michael A. Innis (Editor), et al. (1995) and PCR: Introduction to Biotechniques Series, C. R. Newton, A. Graham (1997)). Amplified polynucleotides are purified by methods well known in the art (e.g., column purification). A polynucleotide is considered pure when it has been isolated so as to be substantially free of primers and incomplete products produced during the synthesis of the desired polynucleotide. Preferably, a purified polynucleotide will also be substantially free of contaminants which may hinder or otherwise mask the binding activity of the molecule.

In the arrays of the invention, the polynucleotide compositions are stably associated with the surface of a solid support, wherein the support may be a flexible or rigid solid support.

Any solid support to which a nucleic acid member may be attached may be used in the invention. Examples of suitable solid support materials include, but are not limited to, silicates such as glass and silica gel, cellulose and nitrocellulose papers, nylon, polystyrene, polymethacrylate, latex, rubber, and fluorocarbon resins such as TEFLON™.

The solid support material may be used in a wide variety of shapes including, but not limited to slides and beads. Slides provide several functional advantages and thus are a preferred form of solid support. Due to their flat surface, probe and hybridization reagents are minimized using glass slides. Slides also enable the targeted application of reagents, are easy to keep at a constant temperature, are easy to wash and facilitate the direct visualization of RNA and/or DNA immobilized on the solid support. Removal of RNA and/or DNA immobilized on the solid support is also facilitated using slides.

The particular material selected as the solid support is not essential to the invention, as long as it provides the described function. Normally, those who make or use the invention will select the best commercially available material based upon the economics of cost and availability, the expected application requirements of the final product, and the demands of the overall manufacturing process.

Numerous methods are used for attachment of the nucleic acid members of the invention to the substrate (a process referred as spotting). For example, polynucleotides are attached using the techniques of, for example U.S. Pat. No. 5,807,522, which is incorporated herein by reference for teaching methods of polymer attachment. Alternatively, spotting is carried out using contact printing technology.

The amount of polynucleotide present in each composition will be sufficient to provide for adequate hybridization and detection of target polynucleotide sequences during the assay in which the array is employed. Generally, the amount of each nucleic acid member stably associated with the solid support of the array is at least about 0.1 ng, preferably at least about 0.5 ng and more preferably at least about 1 ng, where the amount may be as high as 1000 ng or higher, but will usually not exceed about 20 ng. Where the nucleic acid member is “spotted” onto the solid support in a spot comprising an overall circular dimension, the diameter of the “spot” will generally range from about 10 to 5,000 μm, usually from about 20 to 2,000 μm and more usually from about 50 to 1000 μm.

Control polynucleotides may be spotted on the array and used as target expression control polynucleotides and mismatch control nucleotides to monitor non-specific binding or cross-hybridization to a polynucleotide in the sample other than the target to which the probe is directed. Mismatch probes thus indicate whether a hybridization is specific or not. For example, if the target is present the perfectly matched probes should be consistently brighter than the mismatched probes. In addition, if all central mismatches are present, the mismatch probes are used to detect a mutation.

Target Preparation

The targets for the microarrays, are derived from human fluid or tissue samples. It may be desirable to amplify the target nucleic acid sample prior to hybridization. One of skill in the art will appreciate that whatever amplification method is used, if a quantitative result is desired, care must be taken to use a method that maintains or controls for the relative frequencies of the amplified polynucleotides. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high density array may then include probes specific to the internal standard for quantification of the amplified polynucleotide. Detailed protocols for quantitative PCR are provided in PCR Protocols, A Guide to Methods and Applications, Innis et al., Academic Press, Inc. N.Y., (1990). Other suitable amplification methods include, but are not limited to polymerase chain reaction (PCR) (Innis, et al., PCR Protocols. A guide to Methods and Application. Academic Press, Inc. San Diego, (1990)), ligase chain reaction (LCR) (see Wu and Wallace, Genomics, 4: 560 (1989), Landegren, et al., Science, 241: 1077 (1988) and Barringer, et al., Gene, 89: 117 (1990), transcription amplification (Kwoh, et al., Proc. Natl. Acad. Sci. USA, 86: 1173 (1989)), and self-sustained sequence replication (Guatelli, et al., Proc. Nat. Acad. Sci. USA, 87: 1874 (1990)).

The invention provides for labeled target or labeled probe. Any analytically detectable marker that is attached to or incorporated into a molecule may be used in the invention. An analytically detectable marker refers to any molecule, moiety or atom which is analytically detected and quantified. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., ³H, ¹²⁵I, 35S, ¹⁴C, or ³²P), enzymes (e.g., horseradish peroxidase, alkaline phosphatase and others commonly used in an ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Means of detecting such labels are well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The labels may be incorporated by any of a number of means well known to those of skill in the art. However, in a preferred embodiment, the label is simultaneously incorporated during the amplification step in the preparation of the sample polynucleotides. Thus, for example, polymerase chain reaction (PCR) with labeled primers or labeled nucleotides will provide a labeled amplification product. In a preferred embodiment, transcription amplification, as described above, using a labeled nucleotide (e.g. fluorescein-labeled UTP and/or CTP) incorporates a label into the transcribed polynucleotides. Alternatively, a label may be added directly to the original polynucleotide sample (e.g., mRNA, polyA mRNA, cDNA, etc.) or to the amplification product after the amplification is completed. Means of attaching labels to polynucleotides are well known to those of skill in the art and include, for example nick translation or end-labeling (e.g. with a labeled RNA) by kinasing of the polynucleotide and subsequent attachment (ligation) of a polynucleotide linker joining the sample polynucleotide to a label (e.g., a fluorophore).

In a preferred embodiment, the target will include one or more control molecules which hybridize to control probes on the microarray to normalize signals generated from the microarray. Labeled normalization targets are polynucleotide sequences that are perfectly complementary to control oligonucleotides that are spotted onto the microarray as described above. The signals obtained from the normalization controls after hybridization provide a control for variations in hybridization conditions, label intensity, “reading” efficiency and other factors that may cause the signal of a perfect hybridization to vary between arrays.

Hybridization Conditions

Polynucleotide hybridization involves providing a denatured probe or target nucleic acid member and target polynucleotide under conditions where the probe or target nucleic acid member and its complementary target can form stable hybrid duplexes through complementary base pairing. The polynucleotides that do not form hybrid duplexes are then washed away leaving the hybridized polynucleotides to be detected, typically through detection of an attached detectable label. It is generally recognized that polynucleotides are denatured by increasing the temperature or decreasing the salt concentration of the buffer containing the polynucleotides. Under low stringency conditions (e.g., low temperature and/or high salt) hybrid duplexes (e.g., DNA:DNA, RNA:RNA, RNA:DNA, cDNA:RNA and cDNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches. Methods of optimizing hybridization conditions are well known to those of skill in the art (see, e.g., Laboratory Techniques in Biochemistry and Molecular Biology, Vol. 24: Hybridization With Polynucleotide Probes, P. Tijssen, ed. Elsevier, N.Y., (1993)).

Following hybridization, non-hybridized labeled or unlabeled polynucleotide is removed from the support surface, conveniently by washing, thereby generating a pattern of hybridized target polynucleotide on the substrate surface. A variety of wash solutions are known to those of skill in the art and may be used. The resultant hybridization patterns of labeled, hybridized oligonucleotides and/or polynucleotides may be visualized or detected in a variety of ways, with the particular manner of detection being chosen based on the particular label of the test polynucleotide, where representative detection means include scintillation counting, autoradiography, fluorescence measurement, calorimetric measurement, light emission measurement and the like.

Image Acquisition and Data Analysis

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured and compared to a unit value corresponding to the signal emitted by a known number of end labeled target polynucleotides to obtain a count or absolute value of the copy number of each end-labeled target that is hybridized to a particular spot on the array in the hybridization pattern.

Methods for analyzing the data collected from hybridization to arrays are well known in the art. For example, where detection of hybridization involves a fluorescent label, data analysis can include the steps of determining fluorescent intensity as a function of substrate position from the data collected, removing outliers, i.e., data deviating from a predetermined statistical distribution, and calculating the relative binding affinity of the test polynucleotides from the remaining data. The resulting data is displayed as an image with the intensity in each region varying according to the binding affinity between associated oligonucleotides and/or polynucleotides and the test polynucleotides.

Following detection or visualization, the hybridization pattern is used to determine quantitative information about the genetic profile of the labeled target polynucleotide sample that was contacted with the array to generate the hybridization pattern, as well as the physiological source from which the labeled target polynucleotide sample was derived. By genetic profile is meant information regarding the types of polynucleotides present in the sample, e.g. in terms of the types of genes to which they are complementary, as well as the copy number of each particular polynucleotide in the sample.

Diagnostic or Prognostic Tests

The invention provides for diagnostic tests for detecting diseases. The invention also provides for prognostic tests for monitoring a patient's response to therapy. According to the method of the invention, the presence of disease or the patient's response to therapy is detected by obtaining a fluid or tissue sample from a patient. A sample comprising nucleic acid is prepared from the fluid or tissue sample. The nucleic acid extracted from the sample is hybridized to an array comprising a solid substrate and a plurality of nucleic acid members, wherein each member is indicative of the presence of disease or a predisposition to a disease or disorder. According to this diagnostic test, hybridization of the sample comprising nucleic acid to one or more nucleic acid members on the array is indicative of disease, a predisposition to a disease or disorder, or in the case of a prognostic test, indicative of a patient's response to therapy.

Kits

Kits containing reagents and instructions to carry out the methods of the present invention are provided. For example, the kit may comprise reagents and instructions for detecting mitochondrial mutations, heteroplasmies, homoplasmies in tissue specific samples and tissue associated fluids. The kits may also comprise one or more primers which hybridize to the mitochondrial genome for making a primer extension product. Kits may also include a disposable chip, means for holding the disposable chip, means for extraction of mtDNA and means for access to a database of mtDNA sequences.

Other utilities for the present invention, such as that described above and in the following examples, will be readily apparent to those skilled in the art.

The present invention is more particularly described in the following examples which are intended as illustrative only since numerous modifications and variations will be apparent to those skilled in the art.

### EXAMPLE 1

### Prostate Tumours

Following acquisition of prostate fluid or surgery to remove prostate tumours, biopsy slides are prepared to identify transforming or cancerous cells. Laser Capture Microdissection (LCM) microscopy is used to isolate cells that are either normal, benign, or malignant from the tissue section. Procurement of diseased cells of interest, such as precancerous cells or invading groups of cancer cells is possible from among the surrounding heterogeneous cells.

Total DNA extraction from each of these cells was purified according to a modification of the protocol outlined by Arcturus Engineering Inc. DNA was extracted from cells with a 50 μl volume of I mg/ml proteinase K (PK), in IOmM Tris pH 8.0, O.1 mM EDTA pH 8.0, and 0.1% Tween 20, at 42° C. overnight. Following incubation overnight at 42° C. the tubes were removed from the incubation oven. The samples were microcentrifuged for 5 min at 6400 rpm (2000×g). The CapSure™ was removed from the tube and discarded. The tube was incubated at 95° C. for 10 minutes (PK is inactivated) and then cooled to room temperature. 5-50 μl of the sample was used for PCR amplification.

Following purification, individual samples are amplified, by LX-PCR using the appropriate primers for hypervariable region 1 (HV1), hypervariable region 2 (HV2) and the entire 12S region. These PCR products are then sequenced using high throughput methods as is well known in the art.

Alternatively, full length mitochondrial genomes may be amplified using the primers in Table 5. Specific capture and amplification of DNA derived from malignant tumour cells of any Geason Grade, cells from an adjacent benign gland and cells from a “distant” benign gland may be amplified. Other prostate tissues which could and are amplified includes: prostatic intraepithelial neoplasia (PIN), benign prostatic hyperplasia (BPH), hyperplasia of various types, stroma, and cells with undetermined changes. This work was done on prostate tissues from 31 individuals electing to have a prostatectomy because of a prostate cancer diagnosis. Three tissue types were captured: malignant, adjacent benign and distant benign from each individual. Blood from each patient was used as a positive, non-diseased tissue control. Amplification and sequencing of these samples resulted in the novel mutations seen in Table 4. The mutations of Table 4 are also provided in SEQ ID No: 102 which lists the substitutions, SEQ ID NOs: 103 to 109 which lists the deletions, and SEQ ID No: 110 to 138 which lists the insertions. Polymorphisms and mutation positions were determined by comparison to the Revised Cambridge Reference Sequence (2001), however the historical numbering has been maintained such that the deletion at position 3106 is denoted as a gap and the rare polymorphism 750A has been retained. A subset of this data (7 protein coding regions) was then subjected to principal component analysis, as is standard in the art, with the following results as shown in Table 1a:

TABLE 1a

| | | distant | adjacent | | |
|---|---|---|---|---|---|
| | blood | benign | benign | malignant | unknown |
| blood | 100.00% | 0.00% | 0.00% | 0.00% | 0.00% |
| distant | 16.13% | 35.48% | 9.68% | 29.03% | 16.13% |
| benign | | | | | |
| adjacent | 12.9% | 12.90% | 45.16% | 3.22% | 25.81% |
| benign | | | | | |
| malignant | 3.22% | 0.00% | 0.00% | 96.78% | 0.00% |

The results demonstrate a clear pattern of malignant transformation. Normal tissue (blood) and malignant tissue display high clustering frequencies (1.00 and 0.967). Interestingly, adjacent and distant benign, both of which appear normal in a histological and pathological sense, show levels of transformation with over 50% of the samples falling outside the distant benign and adjacent benign intercepts. Moreover, the same data was analyzed by a neural network, as is standard in the art, with the following results as shown in Table 1b:

TABLE 1b

| | | distant | adjacent | |
|---|---|---|---|---|
| | Blood | benign | benign | malignant |
| blood | 100.00% | 0.00% | 0.00% | 0.00% |
| distant | 6.45% | 0.00% | 0.00% | 93.55% |
| benign | | | | |
| adjacent | 19.35% | 0.00% | 0.00% | 77.14% |
| benign | | | | |
| malignant | 3.22% | 0.00% | 0.00% | 96.78% |

This table shows that, in the presence of tumour, all prostate tissue is considered malignant at the molecular level, even though histological appearance of the tissue may be “normal.”

TABLE 1c
MUTATION REGIONS - ND1, ND2, COX1 and CYTB

| | distant benign | adjacent benign | malignant |
|---|---|---|---|
| 1. | 105DB - 6037 | 105AB - 4655 | 105ML - 4655, |
| | | | 4917, 7407 |
| 2. | 208DB | 208AB - 4917, | 208ML - 4722, |
| | | 5174, 5985, 6776 | 6553, 7028 |
| 3. | 349DB | 349AB | 349ML - 6548 |
| 4. | 377DB - 4735, | 377AB | 377ML - 4917, |
| | 5984, 6912 | | 6686, 7028, 7407, |
| | | | 15452, 15607 |
| 5. | 378DB | 378AB - 6776, | 378ML - 4703 |
| | | 15043 | |
| 6. | 380DB - 3469, | 380AB - 4917, | 380ML - 15218 |
| | 3507, 15218, | 4951, 5440, 6059 | |
| 7. | 382DB - 6307, | 382AB - 4716, | 382ML - 6147, |
| | 7028, 7407, 15452, | 5312, 5371, | 6691, 7178, 15523 |
| | 15527, 15607 | 15162, 15323, | |
| | | 15324 | |
| 8. | 384DB - 4733, | 384AB - 6219, | 384ML - 4733, |
| | 7028 | 15452, 15607 | 7028 |
| 9. | 386DB - 15677 | 386AB - 15244, | 386ML |
| | | 15301, 15452, | |
| | | 15607, 15670 | |
| 10. | 416DB - 4561, | 416AB | 416ML - 4864 |
| | 15326, 15525 | | |
| 11. | 417DB | 417AB | 417ML - 4917 |
| 12. | 418DB | 418AB - 7159 | 418ML - 7028, |
| | | | 7407 |
| 13. | 426DB | 426AB | 426ML - 4892, |
| | | | 5102, 5213 |
| 14. | 449DB - 4646, | 449AB | 449ML |
| | 4917, 5999, 6047, | | |
| | 7407 | | |
| 15. | 450DB - 15323 | 450AB - 4917, | 450ML - 3398, |
| | | 5300, 15323 | 5147, 6009, 15323 |
| 16. | 451DB | 451AB - 4217 | 451ML |
| 17. | 452DB - 4018, | 452AB - 3308, | 452ML - 3480, |
| | 6557, 15286 | 3480, 3594, 3666, | 4591, 5268, 7159, |
| | | 3693, 5036, 5046, | 7407 |
| | | 5393, 5984, 6548, | |
| | | 6827, 6989, 7055, | |
| | | 7146, 7256, 7389, | |
| | | 15115 | |
| 18. | 455DB - 7160 | 455AB | 455ML |
| 19. | 456DB - 3589, | 456AB - 4216, | 456ML - 4787, |
| | 4216, 5312, 5424, | 4787, 4917, 7407, | 6579, 7059, 15302 |
| | 6041, 7013, 7407, | 15343, 15452, | |
| | 15384, 15452 | 15607 | |
| 20. | 457DB | 457AB | 457ML |
| 21. | 458DB - 5198, | 458AB | 458ML |
| | 7407 | | |
| 22. | 460DB - 5147 | 460AB | 460ML |
| 23. | 461DB - 7028, | 461AB - 3394, | 461ML - 7184 |
| | 7184 | 7184, 14899 | |
| 24. | 463DB - 14903 | 463AB - 14903 | 463ML - 14903 |
| 25. | 464DB | 464AB | 464ML - 6314, |
| | | | 6643, 6667, 7028, |
| | | | 7066, 7407, 15265 |
| 26. | 466DB - 3507, | 466AB - 3908, | 466ML - 6382, |
| | 7028 | 3969, 3992, 4017, | 6776, 15527 |
| | | 4185, 4239, 7028 | |
| 27. | 467DB | 467AB - 4917, | 467ML |
| | | 5147 | |
| 28. | 498DB - 14903, | 498AB - 14918 | 498ML |
| | 14918, 15355 | | |
| 29. | 501DB - 4580, | 501AB - 4580, | 501ML - 3966, |
| | 4826, 6224, 7007 | 6776 | 4569, 4580, 4917, |
| | | | 15379 |
| 30. | 504DB - 3507 | 504AB | 504ML |
| 31. | 505DB - 15307, | 505AB - 4216, | 505ML |
| | 15526 | 4917, 5456, 6776, | |
| | | 6953, 7028, 7407, | |
| | | 15452 | |

Cluster Analysis

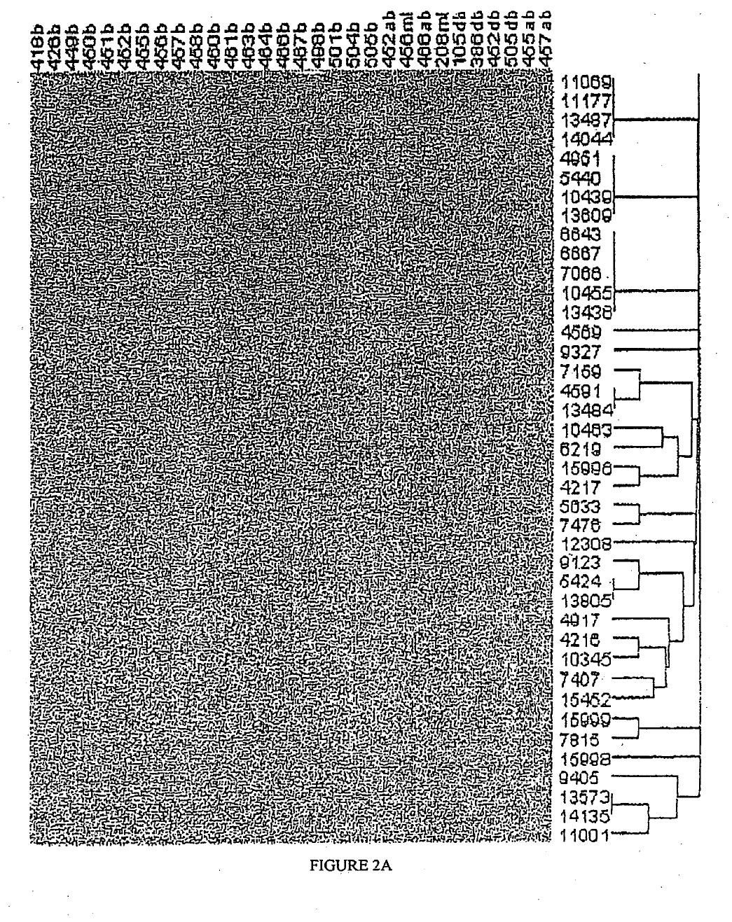

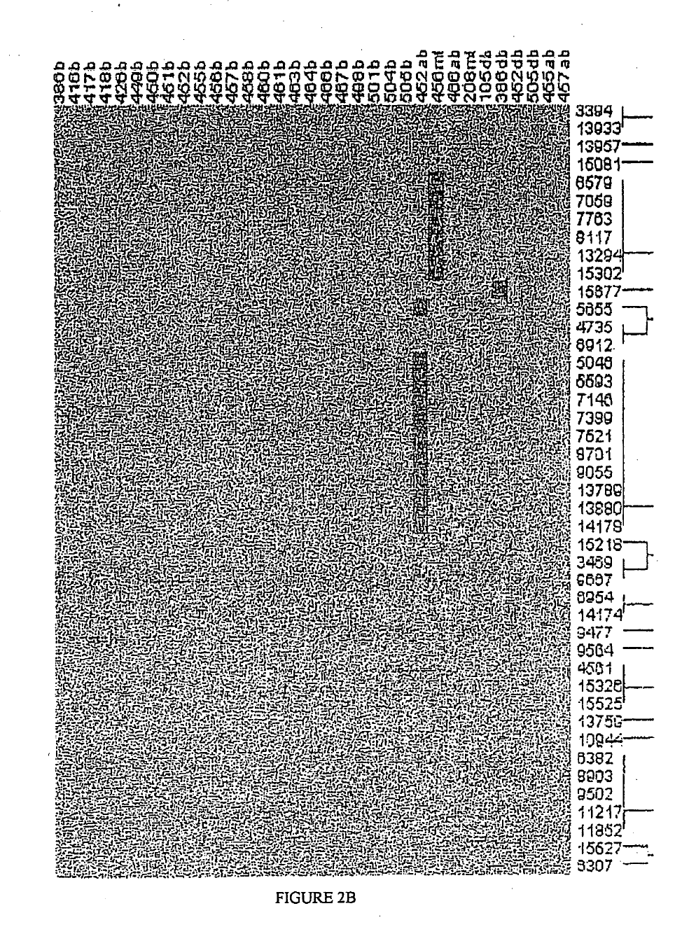

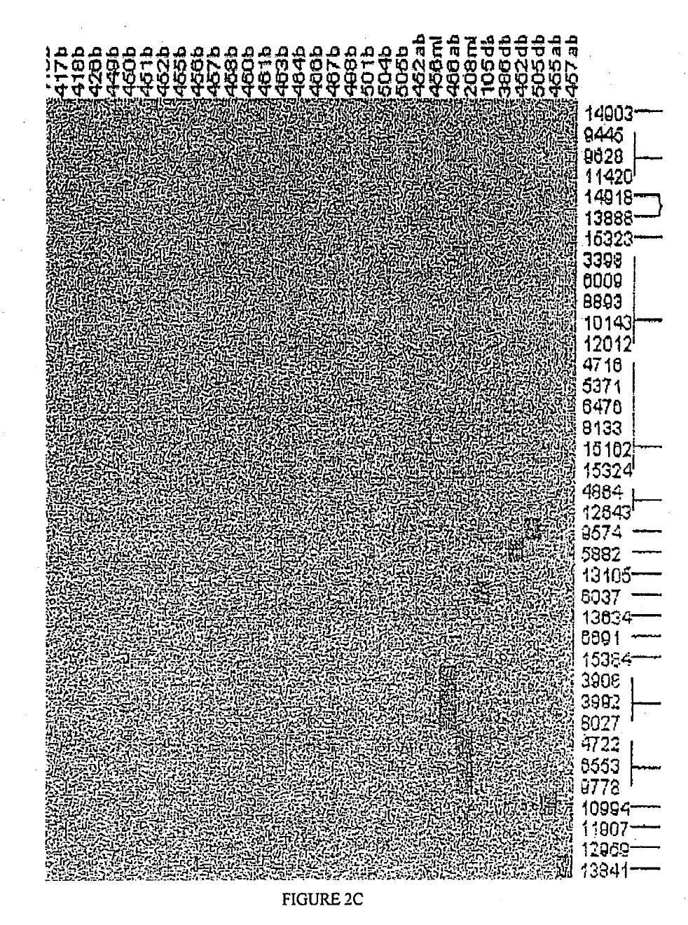

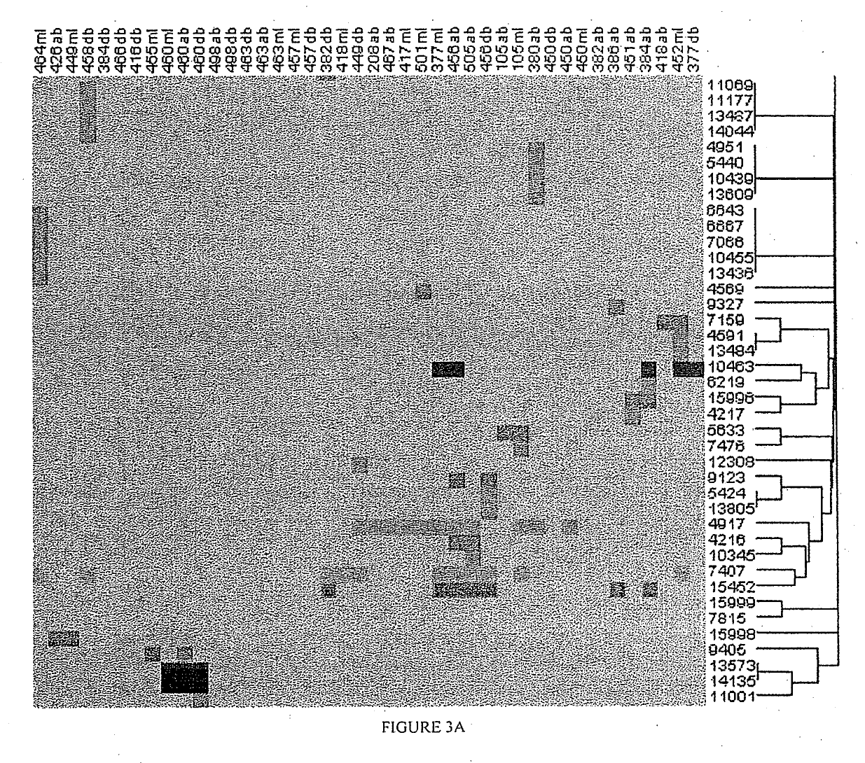

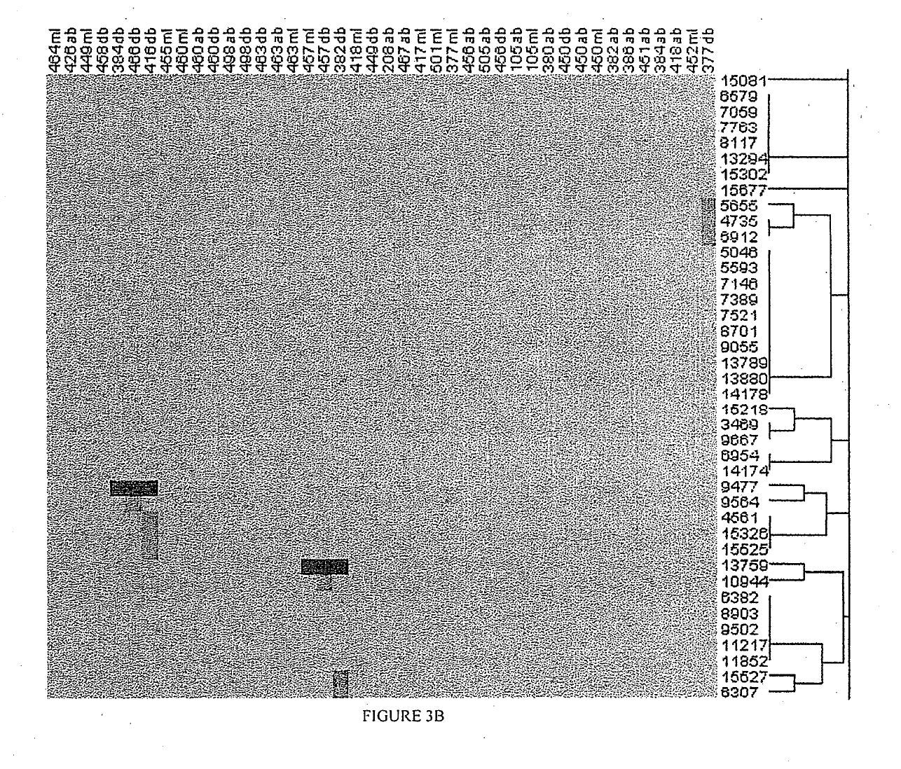

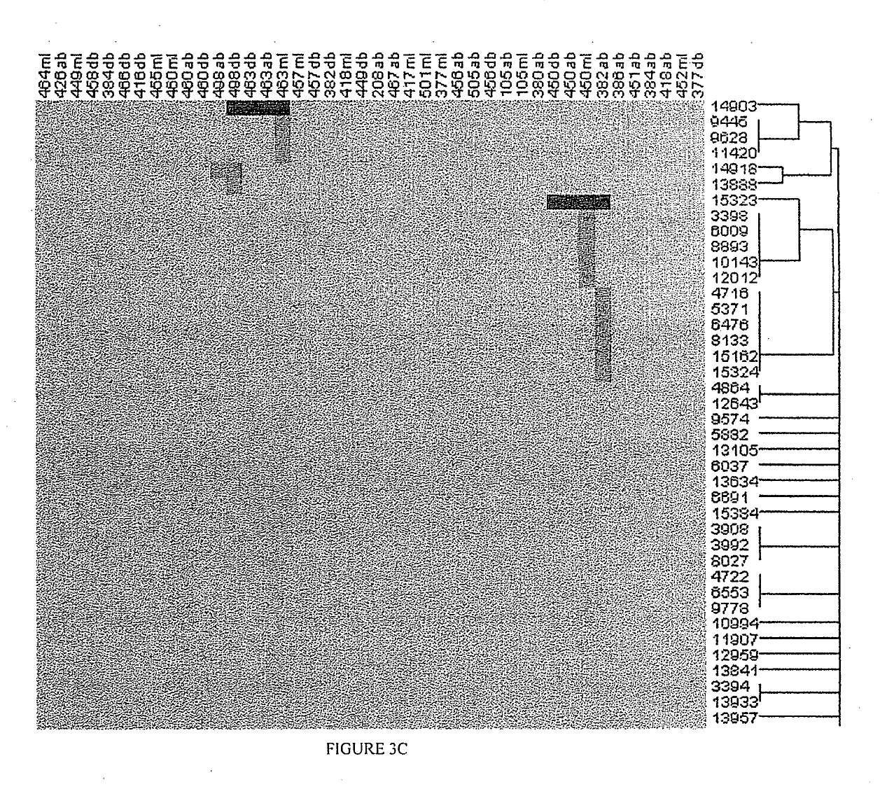

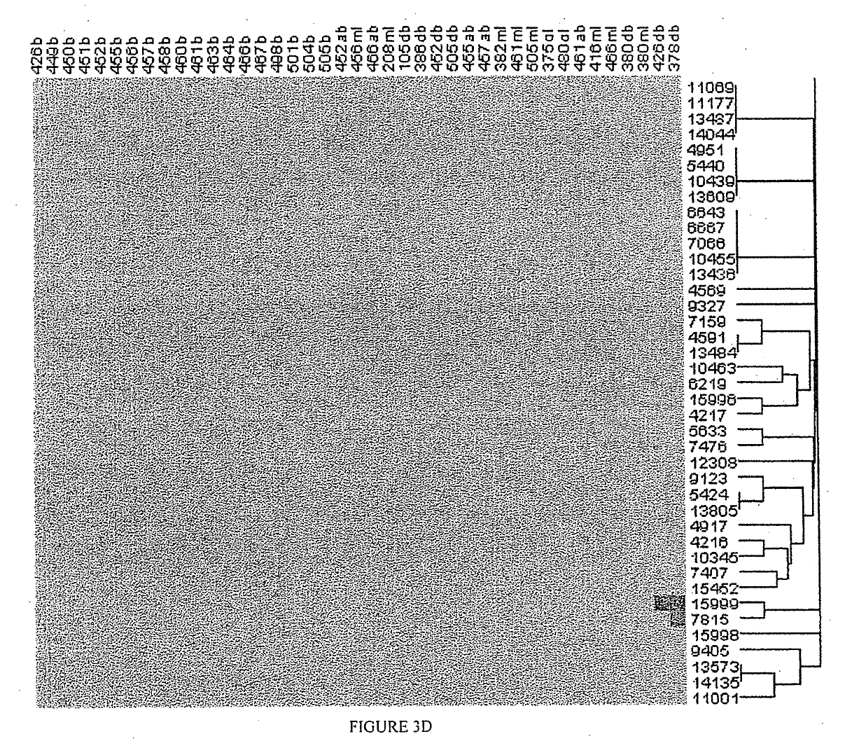

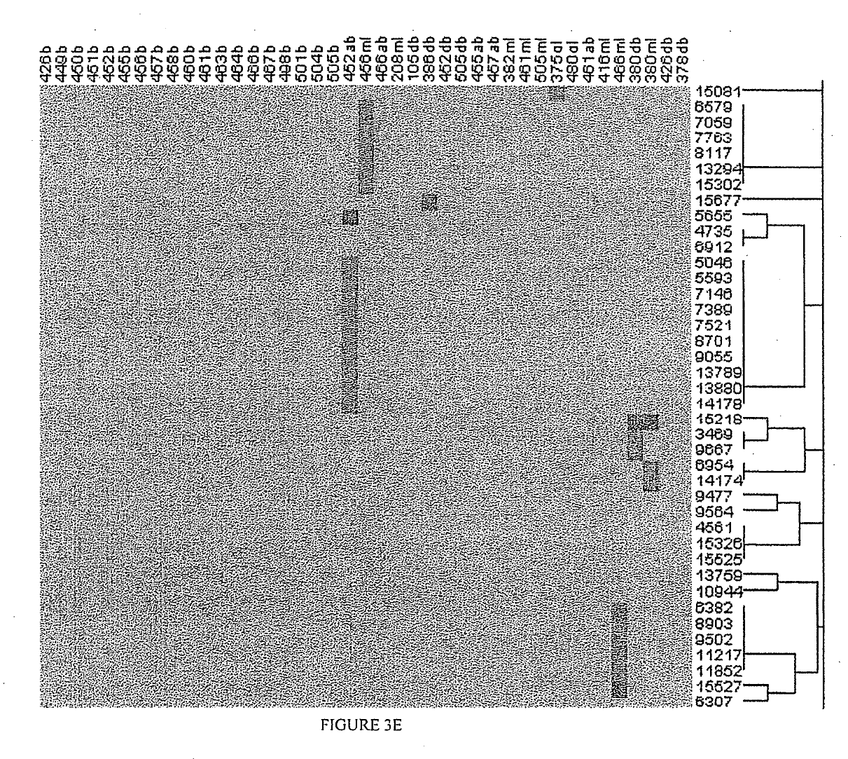

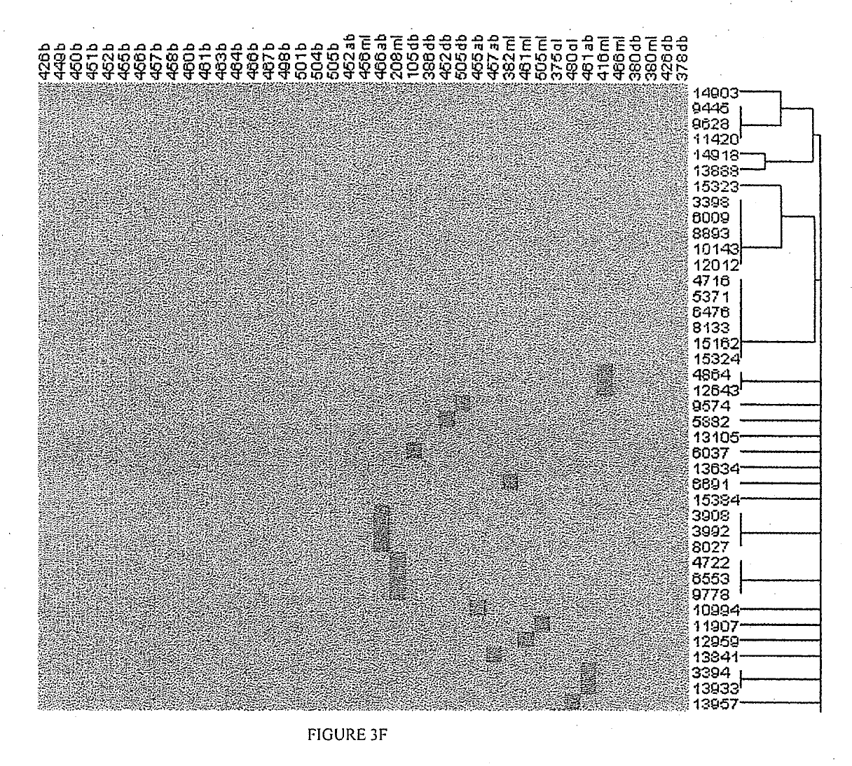

The mutations identified in the 31 individuals diagnosed with prostate cancer were analyzed using Hierarchical Clustering Explorer (HCE) (www.cs.umd.edu/hcil/multi-cluster; Seo et al, 2002; Seo et al. 2003; Zhao et al 2003; Seo and Shneiderman; Seo et al; 2004a; Seo et al. 2004b; Seo et al. 2005a; Seo et al. 2004c; Seo et al. 2005b). In addition, the mitochondrial genomes from prostate needle biopsy tissue of 12 males clinically symptomatic for prostate cancer, but where pathology results indicated that the prostate tissue was not malignant, were also analyzed. FIGS. 2 and 3 show the cluster analysis of the identified non-synonymous mitochondrial mutations. FIG. 2 is the first half of the cluster analysis and FIG. 3 is the second half of the cluster analysis. The y-axis lists the patient numbers for the 31 individuals with prostate cancer (105, 208, 349, 377, 378, 380, 382, 384, 386, 416, 417, 418, 426, 449, 450, 451, 452, 455, 456, 457, 458, 460, 461, 463, 464, 466, 467, 498, 501, 504, 505) and the 12 individuals showing clinical symptoms but having no malignancy as determined by pathology (2, 35, 51, 209, 270, 278, 375, 480, 503, 536, 560, 858). The y-axis also indicates the source of the tissue (i.e. distant benign (db), adjacent benign (ab), malignant (ml), and (b) blood). Benign glandular tissue from symptomatic but non-malignant tissue of the 12 individuals is indicated by “gl”. The x-axis lists the sites of the mutations.

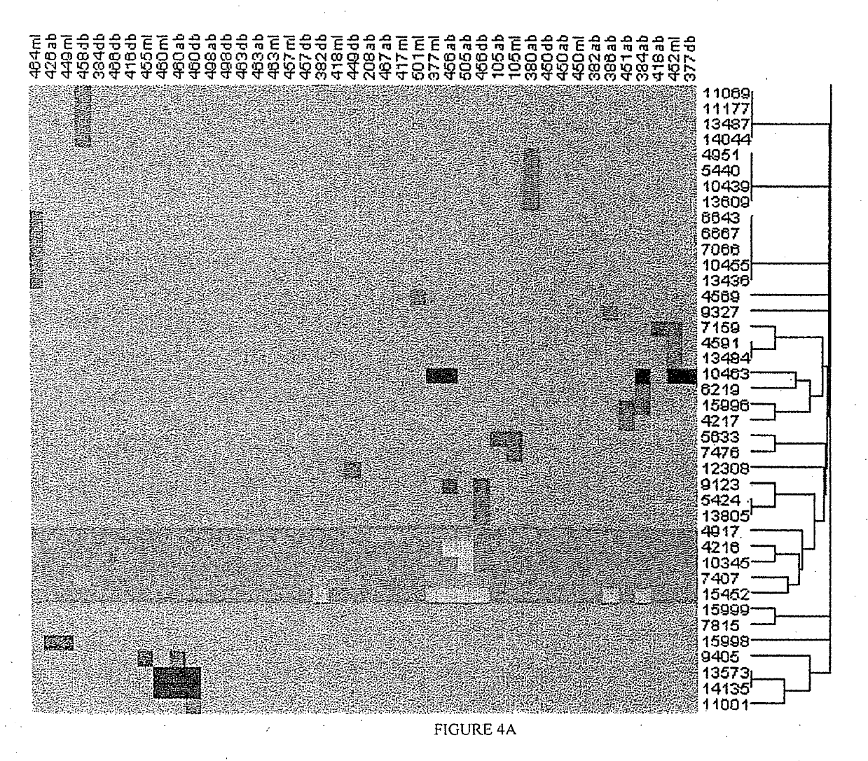

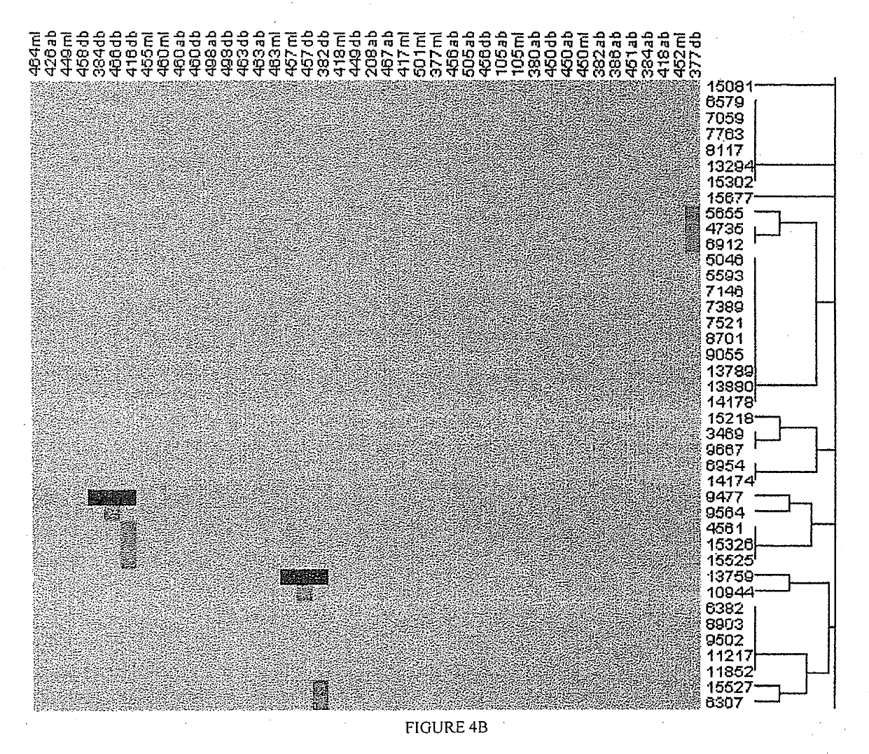

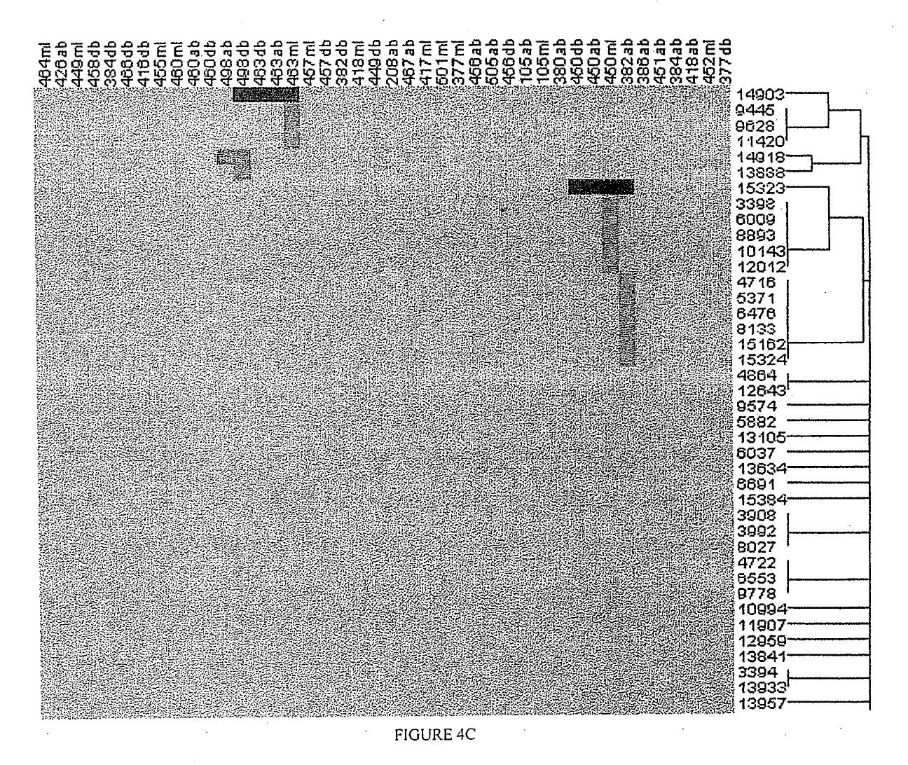

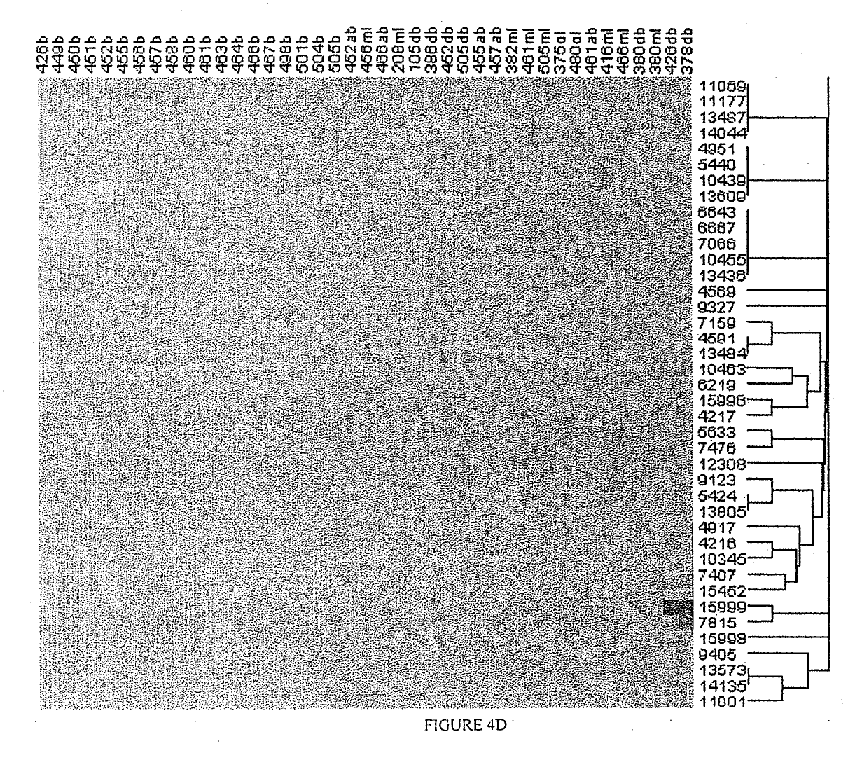

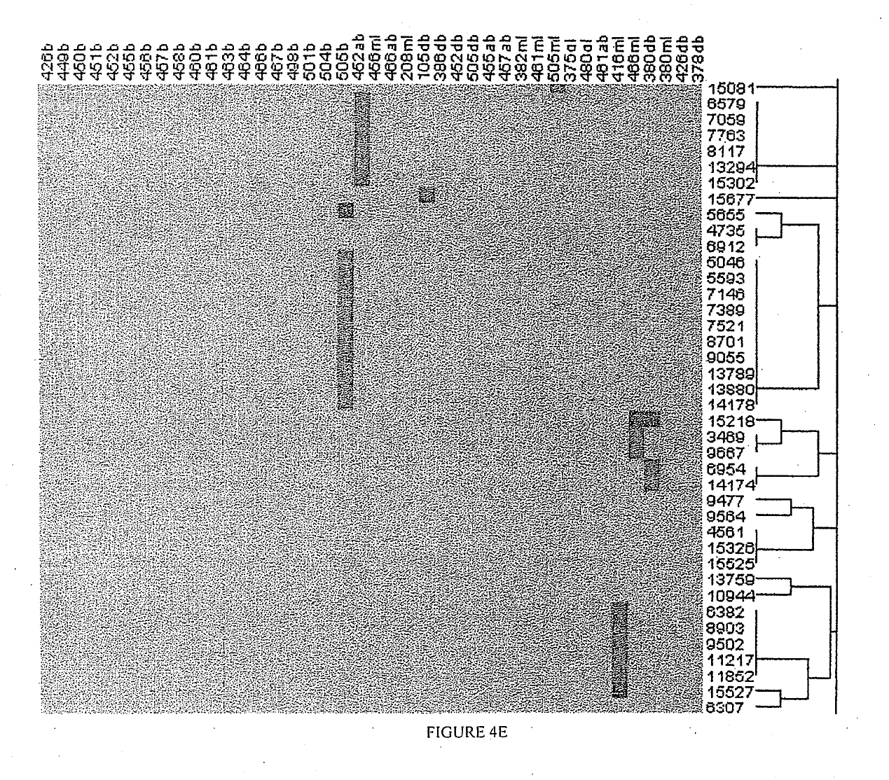

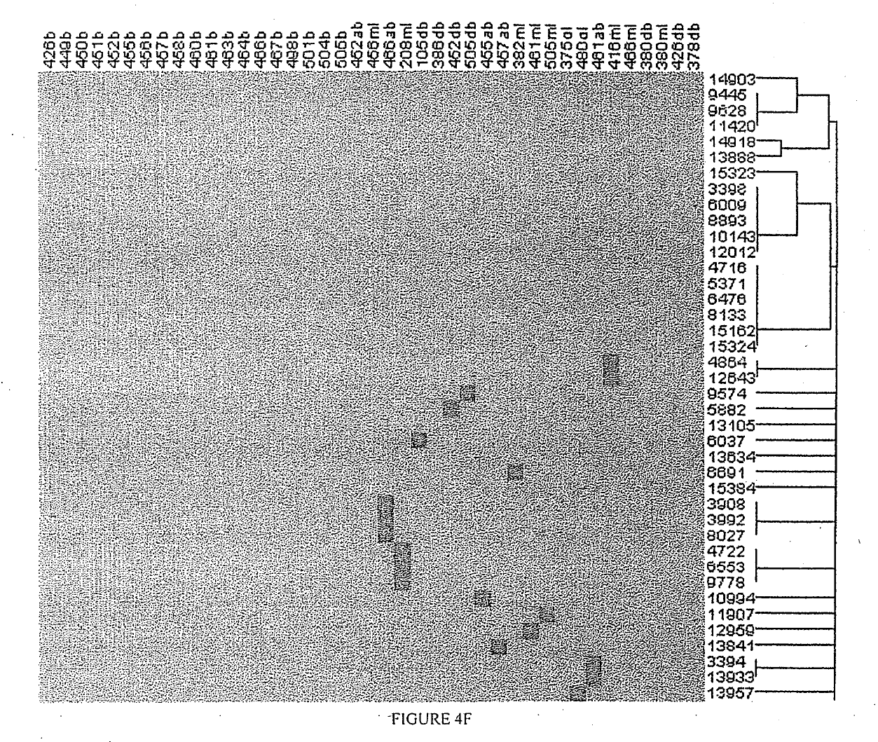

FIG. 4 is a copy of FIG. 3 showing a suite of non-synonymous mutations associated with clinical prostate cancer in the shaded area. The mutations occur in specific genes: Mutations at positions 4216 and 4217 both occur in the ND1 gene (NADH dehydrogenase subunit 1). The mutation at position 4917 occurs in the ND2 gene (NADH dehydrogenase subunit 2). The mutation at position 7407 occurs in the COXI gene (cytochrome c oxidase subunit 1). Finally, the mutation at position 15452 occurs in the CytB gene (cytochrome b). Mutations 4216 and 4217 invoke a non-synonymous mutation at the same amino acid, and like 4917, are secondary mutations associated with Leber's Hereditary Optic Neuropathy (LHON) (Mitochondrial Research Society; Huoponen, 2001; MitoMap). Other than prostate cancer, an association for 7407 remains unattested. A mutation at 15452 was found in 5/5 patients with ubiquinol cytochrome c reductase (complex III) deficiency (Valnot et al. 1999).

In the cohort of 31 men who underwent a prostatectomy for prostate cancer these mutations, one or more, were noted in 20 of the subjects (64.5%). As mentioned above, in each of these individuals, total mitochondrial genome sequences were obtained from prostate cross sections for three types of associated tissues: malignant, benign tissue adjacent to malignant tissue and “distant” benign tissue, removed from any surrounding tissue pathology. Tissues were laser capture microdisssected (LCM) by a qualified, clinical pathologist. Sequences were compared to mitochondrial DNA (mtDNA) extracted and sequenced from the patient's blood. Sequencing results indicate that mitochondrial mutations, often heteroplasmic, appear in all three tissue types. Contrary to the analysis of Alonso et al. (2005), mutations in mitochondrial DNA from malignant tissue, adjacent benign and distant benign were found in comparison with mtDNA sequences from the patient's blood. Heteroplasmy, mutant and normal mitochondrial genomes existing within the same individual, is considered evidence of recent mutation (Huoponen, 2001). Most mutations are not held in common between the three tissue types from the same individual. In fact, the comparative mutation loads for each tissue type is roughly the same, indicating that mtDNA mutations are active within the prostate in the presence of tumour, irrespective of prostate tissue type. Moreover, tissue which appears histologically benign, is often found with mutations in the mitochondrial genome. These mutations are not associated with mitochondrial sequences embedded in the nucleus, or NUMTS (nuclear/mitochondrial sequences) (Lopez, 1994). A lengthy cloning and sequencing study of human NUMTS utilizing a Rho⁰ cell line, as well as comparison to known NUMTS archived in the NCBI database was done and no mutations were detected to known mitochondrial pseudogenes archived in public databases to ensure data clean of pseudogene data points.

In the 12 males clinically symptomatic for prostate cancer, wherein the pathology results indicated that the prostate tissue was not malignant, benign glandular tissue was recovered by LCM and the mitochondrial genomes amplified and sequenced from prostate needle biopsy tissues. Results indicate a complete absence of mtDNA mutations in four patients, mutations only in the non-coding control region in four patients, or mutations in both the control region and gene coding regions in four patients. However, the mutation locations were significantly different than the malignant cohort (P>0.01). Additionally, only one coding region mutation was observed in these individuals and of these mutations one fell into the regions mentioned above (i.e. NDI, ND2, COXI and CytB) (patient 375 CytB at position 15081).

When both synonymous and non-synonymous mutations, inclusive of the four protein coding regions, were used as malignant disease markers, 30/31 (97%) of the prostate cancer group was identified. Table 1c lists the synonymous and non-synonymous mutations found in ND1, ND2, COX1, and CytB in tissue samples from the 31 individuals with prostate cancer. Mutations in ND1, ND2, COX1, and CytB are clearly associated with prostate cancer. Although the present invention has identified the mutations listed in Table 1c and FIGS. 2 to 4, the invention includes any mutations in ND1, ND2, COX1 and CytB. In the same analyses, 1 of the 12 benign, symptomatic patients was included with the malignant group; however, this patient had a mutation in CytB, which may indicate early cancer progression. The rest of this group clustered with the blood, or normal control.

A statistical analysis of distant benign from malignant subjects compared to the benign glands of the 12 symptomatic but not malignant patients was done. Since there were 31 malignant patients and 12 symptomatic but not malignant subjects, random samples of 6 groups of 12 from the malignant patients were run against the 12 symptomatic but not malignant subjects. A chi square analysis for each of the 6 groups was run. The results are listed in Table 1d. In every case, the differences were significant at 0.01 level, meaning that the difference between mitochondrial sequences from distant benign tissue in malignant subjects and mitochondrial sequences from symptomatic but not malignant subjects was not due to chance 99% of the time. The analysis was based on the number of mutations found in the four coding regions discussed above.

TABLE 1d
Chi-square test

| Tissues | ND1 | ND2 | COI | CYTB | Xi-Square |
|---|---|---|---|---|---|
| Symptomatic | 0 | 0 | 0 | 1 | |
| First set | 10 | 8 | 12 | 7 | 35.14 |
| Second set | 3 | 6 | 14 | 9 | 30.11 |
| Third set | 4 | 2 | 7 | 8 | 19.12 |
| Fourth set | 3 | 4 | 7 | 11 | 23.09 |
| Fifth set | 4 | 5 | 10 | 8 | 25.12 |
| Sixth set | 4 | 1 | 5 | 9 | 17.11 |

Symptomatic Patients - 12; Distant Benign - 6 random sets of 12 each
1 First set of DB - 426, 456, 450, 449, 380, 501, 386, 456, 466, 378, 457, 504
2 Second set of DB - 377, 105, 451, 417, 501, 456, 426, 498, 382, 461, 384, 464
3 Third set of DB - 467, 208, 466, 505, 452, 458, 105, 498, 380, 463, 208, 377
4 Fourth set of DB - 416, 504, 457, 466, 382, 452, 460, 450, 498, 386, 418, 501
5 Fifth set of DB - 456, 452, 461, 505, 464, 460, 378, 455, 417, 505, 449, 349
6 Sixth set of DB - 463, 426, 498, 349, 455, 466, 452, 380, 386, 461, 450, 460

### EXAMPLE 2

### Duplications in the Non-Coding Region of mtDNA from Sun-Exposed Skin

DNA was extracted from tissue samples as described in Example 1, with the use of DNeasy™ kit supplied by Qiagen. A “back to back” primer methodology was used to investigate the incidence of tandem duplications in the non-coding region (NCR) in relation to sun-exposure. 32 age-matched, split human skin samples, from sun-exposed (n=24) and sun-protected body sites (n=10) were investigated.

The following duplication primers from Brockington et al 1993 and Lee et al 1994 were used:

SEQ ID NO: 1

| | | | |
|---|---|---|---|
| C | L336 | AAC ACA TCT CTG CCA AAC CC | 20 mer |

SEQ ID NO: 2

| | | | |
|---|---|---|---|
| D | H335 | TAA GTG CTG TGG CCA GAA GC | 20 mer |

SEQ ID NO: 3

| | | | |
|---|---|---|---|
| E | L467 | CCC ATA CTA CTA ATC TCA TC | 20 mer |

SEQ ID NO: 4

| | | | |
|---|---|---|---|
| F | H466 | AGT GGG AGG GGA AAA TAA TG | 20 mer |

Primers pairs C/D and E/F are ‘back to back’ at the site of two separate sets of direct repeats in the non-coding region. As a result they only generate a product if a duplication is present at these points. Products generated are 260 bp and/or less common 200 bp variant. Modified PCR conditions are: 100 ng total cellular DNA, 200 μM dNTPs, 2.5 U HotStarTaq polymerase and PCR buffer (Qiagen, Uk), 25 pmoles of primers: one cycle of 94° C. for 4 minutes, 36 cycles of 94° C.×1 minute, 55° C.×1 minute, 72° C.×1 minute and one cycle of 72° C.×7 minutes.

An increased incidence of duplications with increasing sun-exposure was observed, with duplications identified in 10/24 but 0/10 samples from sun-exposed and sun-protected skin respectively (Fisher's exact test, p=0.01 5) (Birch-Machin and Krishnan 2001). The sizes of the most frequent duplications were 200 and 260 base pairs. Interestingly these same samples also contained high levels (>|%) of the 4977 bp common mtDNA deletion as determined by an established quantitative 3-primer PCR assay described in Example 6.

### EXAMPLE 3

### Mutation Fingerprint of mtDNA in Human NMSC and its Precursor Lesions

DNA was extracted from human skin tissue samples as described in Example 1, with the use of DNeasy™ by Qiagen Using specific primers, mtDNA is amplified by PCR and following DNA sample preparation (Qiagen), mutations are identified by automated sequencing (PE Applied Biosystems) using BigDye™ Terminator Cycle sequencing. This methodology is described in Healy et al. 2000; Harding et al. 2000. The entire 16,569 bp human mitochondrial genome is sequenced using established PCR primer pairs, which are known not to amplify pseudogenes, or other nuclear loci. Any putative DNA changes are confirmed by comparison to the revised “Cambridge” human mtDNA reference (Andrews et al. 1999). The sequences obtained from the tumour mtDNA are first compared for known polymorphisms (Andrews et al. 1999; MITOMAP) and then compared with the mtDNA sequence from the normal perilesional skin to identify genuine somatic mutations.

DHPLC is performed on the WAVE™ DNA Fragment Analysis System (Transgenomic, Omaha, USA) which provides a fully automated screening procedure. The same technology is used to screen for heteroplasmic mutations in the skin tumour mtDNA.

Using the back to back primer methodology described in Example 2, the pattern of DNA length mutations (i.e. tandem duplications) in the hypervariable segments of the non-coding region (NCR) are rapidly screened.

### EXAMPLE 4

### Deletion Spectrum of the Entire Mitochondrial Genome in Human NMSC and its Precursor Lesions

MtDNA damage in squamous cell carcinomas (SCCS), Basal cell carcinomas (BCCS) and putative precursor lesions such as Bowen's disease and actinic keratoses (As) was compared to adjacent perilesional skin taken from different sun-exposed body sites. A long-extension PCR technique (LX-PCR) (Ray et al. 1998) was used to amplify the entire mitochondrial genome in order to determine the whole deletion spectrum of mtDNA. A myriad of specific deletions have been observed to occur in the mitochondrial genome. Not all deletions will correlate with non-melanoma skin cancer; however, for an accurate diagnostic method, those deletions that are associated with the disease must be known.

DNA is extracted by use of a commercial kit (Qiangen) according to the manufacturer's recommendations. The entire mitochondrial genome is amplified in two separate reactions using the Expand TM Long Template PCR System™ (Boehringer Manheim, Switzerland). The PCR primers used are those described by Kleinle et al. (1997) covering the following regions of the Cambridge sequence (Andrews et al. 1999): DIA(nucleotides (nt) 336-363), DIB (nt 282-255), OLA (nt 5756-5781), and OLB (nt 5745-5781). These large products eliminate amplification of nuclear pseudogenes. The sequences of the primers are as follows:

| | |
|---|---|
| DIAF: (336-363) | |
| 5′ AACACATCTCTGCCAAACCCCAAAAACA 3′ | SEQ ID NO: 5 |
| OLBR: (5745-5721) | |
| 5′ CCGGCGGCGGGAGAAGTAGATTGAA 3′ | SEQ ID NO: 6 |
| OLAF: (5756-5781) | |
| 5′ GGGAGAAGCCCCGGCAGGTTTGAAGC 3′ | SEQ ID NO: 7 |
| DIBR: (282-255) | |
| 5′ ATGATGTCTGTGTGGAAAGTGGCTGTGC 3′ | SEQ ID NO: 8 |

Amplifications are performed in 50 microlitre reactions containing 16 pmol of each primer, 500 μmol dNTPs, 10×PCR buffer with 22.5 mM MgCl₂ and detergents(kit), 0.75 μl of enzyme (3.5×10³ units/ml) and 50-200 ng of total DNA. One reaction generates 11,095 bp segments of the genome, while another results in 5,409 bp lengths (e.g. Kleinle et al, 1997). The PCR amplification conditions consists of a denaturing stage at 93° C. for 1 min 30 s, followed by 10 cycles of 93° C. for 30 s, 60° C. for 30 s and 68° C. for 12 min, followed by a further 20 cycles of the same profile with an additional 5 s added to the elongation time every cycle. There is a final cycle of 93° C. for 30 s, 60° C. for 30 s and an elongation time of 68° C. for 26 minutes. To ensure reproducibility, a known amount of DNA is separated on a 1% agarose gel and only samples which have at least the same amount of DNA are included in the analysis.

A greater mean number of deletions is found with increasing UV exposure in the tumour samples, as shown in Table 2.

TABLE 2
Comparison of the mean number of deletions observed in the LX-PCR of mtDNA
between normal and tumour skin taken from different UV-exposed body sites.

| | Mean number of deletions in | Mean number of deletions in |
|---|---|---|
| UV exposure | adjacent normal epidermis | epidermal tumour |
| Constant (n = 5) | 1.0 | 3.6 |
| Intermittent (n = 2) | 0 | 1.5 |
| Sun-protected (n = 2) | 0 | 0 |

### EXAMPLE 5

### Aging and MtDNA

Using temporal maternal line comparisons (i.e. great-grandchild through great-grand parents), the entire sequence of mtDNA extracted from a given tissue is rapidly, and accurately sequenced, in order to definitively state the arrangement of nucleotide base pairs for that specific molecule and possible changes through time. These characterizations are compared to health status, aging indicators and between specific maternal lines, within larger populations. This combined information allows crucial statistical discrimination between separate causes resulting in the same mutation/deletion and establishes that the mtDNA sequences, used as a bio-marker, has the required index of specificity and sensitivity in order to establish its validity. In addition, the proportions of base pair deletions and mutations are compared for consistency in various tissues across the 4 maternal generations. Recent methodological developments have permitted detection of base pair deletions implicated in aging in blood samples (Bassam et al. 1991) and have raised the possibility that blood samples may be used to study mtDNA in lieu of skeletal muscle (von Wurmb et al. 1998). After establishing the efficacy of employing leukocytes in lieu of muscle tissue, as representative of mtDNA deletions and/or mutations, the next step measures only mtDNA in leukocytes. MtDNA deletions/mutations are then determined as previously described.

Skeletal muscle or leukocytes are obtained from a patient. DNA is extracted as set out in Example 1. The following primers were used:

| | |
|---|---|
| 12ST1: (1257-1279) | |
| 5′ TATACCGCCATCTTCAGCAAAC3′ | SEQ ID NO: 9 |
| 12ST2: (1433-1411) | |
| 5′ TACTGCTAAATCCACCTTCGAC 3′ | SEQ ID NO: 10 |
| D1F: | |
| 5′ CCTTACACTATTCCTCATCACC 3′ | SEQ ID NO: 11 |
| D1R: | |
| 5′ TGTGGTCTTTGGAGTAGAAACC 3′ | SEQ ID NO: 12 |

Amplifications were performed in 50 microlitre reactions containing 2.0 μmol of each primer, 250 μmol dNTPs, 10×PCR buffer(Thermopol Reaction Buffer), bovine serum albumin, 0.5 units Deep vent polymerase and 50-200 ng of total DNA. The PCR amplification conditions consists of a denaturing stage at 95° C. for 5 min (hot start), followed by 30 cycles of 94° C. for 30 s, 60° C. for 60 s and 72° C. for 30 s with a final extension at 72° C. for 10 min. Gel electrophoresis was performed on a 2% agarose gel at 125 volts for 60 min, stained with ethidium bromide, and visualized under UV light. To ensure reproducibility, a known amount of DNA was separated on a 2% agarose gel and only samples which have the same amount of DNA were included in the analysis.

### EXAMPLE 6

### Quantitative Detection of the 4977 bp Common mtDNA Deletion by 3-Primer PCR

Where appropriate the incidence of the common deletion is determined in a quantitative manner by a 3-primer PCR method which detects levels greater than 1-5% or a dilution PCR method which detects levels less than 1% down to 104%. (See Example 7) Samples are obtained and DNA extracted as described in Example 1. To simultaneously detect and quantify the ratios of both deleted and wild type (wt) mtDNAs in the DNA samples, a 3-primer PCR procedure is used (as described in Birch-Machin et al 1998). Primers A, and C correspond to heavy strand positions 13720-13705 and 9028-9008 respectively (Anderson et al., 1981); primer B corresponds to light strand positions 8273-8289. Primer C maps to a mtDNA region within the common deletion, whereas primers A and B flank the deleted region. Therefore primers B and C only amplify wt-mtDNAs and primers A and B only amplify deleted mtDNAs (the distance between the two primers in the absence of the deletion, approximately 5.5 kb, is too long to be amplified under our PCR conditions as described below). value of zero is obtained for both total mtDNA and deleted mtDNA and the corresponding dilution values are used to calculate the percentage of common deletion in the sample thus:

$$\% common deletion = \frac{total mtDNA dilution {factor}^{( IOD Zero )} \times 100}{common deletion dilution {factor}^{( IOD Zero )}}$$

### EXAMPLE 8

### Denaturing High Performance Liquid Chromatography (DHPLC)

Samples are obtained and DNA extracted as in Example 1. PCR in 13 overlapping fragments using two different PCR conditions as described by van den Bosch et al. (2000). The following three mtDNA specific primer pairs for PCR:

i. Oligo Sequence

| | | |
|---|---|---|
| Mt3118F | CCCTGTACGAAAGGACAAGAG | SEQ ID NO: 13 |
| Mt3334R | TGAGGAGTAGGAGGTTGG | SEQ ID NO: 14 |
| Mt8207F | CCCATCGTCCTAGAATTAATTCC | SEQ ID NO: 15 |
| Mt8400R | ATGGTGGGCCATACGGTAG | SEQ ID NO: 16 |
| Mt14427F | CCCATGCCTCAGGATACTCCTC | SEQ ID NO: 17 |
| Mt14997R | GCGTGAAGGTAGCGGATG | SEQ ID NO: 18 |

The 1-2 kb PCR products are digested into fragments of 90-600 bp and resolved at their optimal melting temperature. Mutations are represented as two peaks and mutations with low percentages, such as <2% heteroplasmy as a “shoulder” in the peak.

DHPLC is performed with a mobile phase consisting of two eluents (pH 7.0). Buffer A contains triethylammonium acetate (TEAA), which interacts with both the negatively charged phosphate groups on the DNA as well as the surface of the column. Buffer B contains TEAA with 25% of the denaturing agent acetonitrile. Fragments were eluted with a linear acetonitrile gradient at a constant flow rate. Increasing the concentration of acetonitrile will denature the fragments. Table 3 below is an example of a standard method for DHPLC of a PCR reaction generated using the WAVEMAKER software (Transgenomics) according to manufacturer's instructions.

Using three primers allowed the simultaneous detection of two bands, the larger one (755 bp) corresponding to the wt-mtDNA, and the smaller one (470 bp) corresponding to deleted mtDNA harbouring the ‘common deletion’. The PCR reaction mixture (25 μl total volume) contained 100 ng total cellular DNA, 200 μM dNTPs, 10 mM Tris-HCl (pH 8.8), 50 mM KCl, 1.5 mM Mg Cl₂, 0.1% Triton X-100, 2.5 U Taq DNA polymerase (BioTaq, BiolineUK Limited, London), 25 pmoles of primers A and B, 6.25 pmoles of primer C and 3 μCi of [α-³²P]-dATP. The PCR conditions were 25 cycles of 94° C. at 1 minute, 55° C. at 1 minute, 72° C. at 2 minutes including a final extension of 15 minutes at 72° C. These PCR products were then electrophoresed through a 6% nondenaturing polyacrylamide gel and the radioactive PCR fragments were quantified by phophorimage analysis using the ImageQuant™ software (Molecular Dynamics, Chesham UK).

### EXAMPLE 7

### Serial Dilution PCR Method to Quantitatively Detect Low Levels (<1%) of the Common mtDNA Deletion

A semi-quantitative PCR method (Corral-Debrinski et al 1991) is used to estimate the proportion of the common deletion in the total mtDNA extracted from the tissue/cell samples. Biological samples are obtained and DNA extracted as described in Example 1. The DNA sample is initially linearised using the restriction enzyme Bam Hi (1 μl enzyme and 1 μl of commercially supplied buffer) at 37° C. for 90 minutes. Serial dilutions are performed in two-fold steps (for total mtDNA there was an initial 10-fold dilution) and PCR performed for each dilution (1 μl) using the following primers:
Primers for total mtDNA
L3108 (nt3108-3127)
H3717 (nt3717-3701)
Primers for Common Deletion
L8282 (nt8282-8305)
H13851(nt13851-13832)

The reaction conditions are as follows:

One cycle 94° C. for 2 minutes, 34 cycles of 94° C. for 45 seconds, 51° C. for 30 seconds (total mtDNA), 56° C. for 30 seconds (common deletion), 72° C. for 1 minute and one final cycle of 72° C. for 8 minutes. All PCR reactions are carried out in the following mixture (50 μl): Sample DNA 1 μl, 0.6 μM forward primer, 0.6 μM reverse primer, 0.2 mM dNTP's, 5 μl GeneAmp® 10×PCR Buffer, (Perkin Elmer), 0.21 μl Amplitaq® DNA polymerase (Perkin Elmer), 35.75 μl sterile autoclaved double distilled water.

Following electrophoresis the PCR productes are visualised on a UV transilluminator (TMW-20, Flowgen Ltd., Lichfield, UK) and a digital image of the gel obtained using image acquisition apparatus (Alpha Imager 2000, Alpha Innotech Corporation, supplied by Flowgen Ltd., Lichfield, UK). The associated image analysis software (Alpha Ease v3.3, Alpha Innotech Corp.) allows the calculation of the integrated optical density (IOD) for each PCR product in a dilution series. The band where an IOD

TABLE 3
Standard Method for DHPLC

| | | | | MI/min |
|---|---|---|---|---|
| Step | Time | % A (buffer) | % B (buffer) | (flow rate) |
| Loading | 0.0 | 52 | 48 | 0.90 |
| Start Gradient | 0.1 | 47 | 53 | |
| Stop Gradient | 4.1 | 39 | 61 | |
| Start Clean | 4.2 | 0 | 100 | |
| Stop Clean | 4.7 | 0 | 100 | |
| Start Equilibrate | 4.8 | 52 | 48 | |
| Stop Equilibrate | 6.8 | 52 | 48 | |

The temperatures for successful resolution of the various heteroduplexes are detailed below and can simply be substituted into the relevant places in Table 2:

| Fragment | Melting temp (° C.) | Gradient of % Buffer B |
|---|---|---|
| Mt3118F | 59 | 51-59 |
| Mt8207F | 58 | 50-58 |
| Mt14427F | 56 | 60-68 |

### EXAMPLE 9

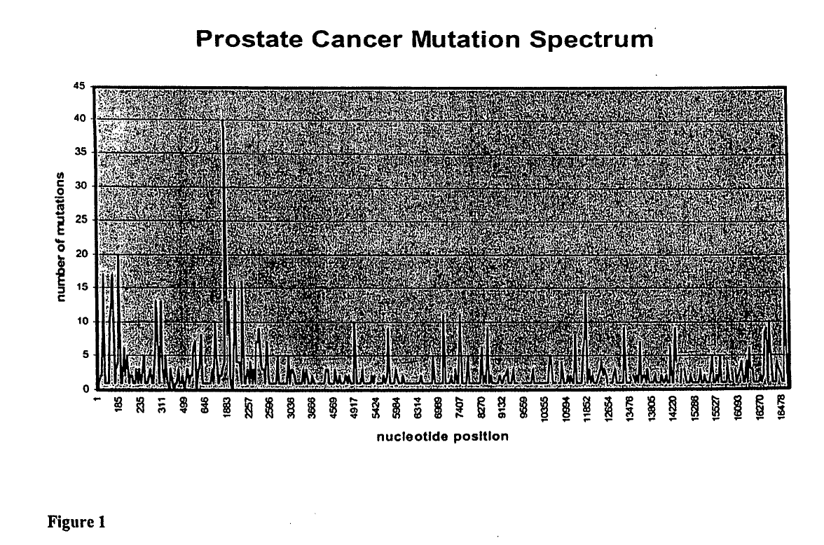

An extensive survey of mtDNA D-loop sequences from 49 prostate needle biopsy patients (46 diagnosed with malignancy) demonstrated mtDNA mutations in all prostatic tissues inclusive of benign prostatic hyperplasia (BPH), available Gleason grades and stroma as compared with the mitochondrial DNA of the patients blood. Moreover, an expanded study of mitochondrial genomes from 31 prostatectomy patients demonstrates equivocable hyper-mutation (Chen et al. 2002; Chen et al. 2003) loads in matched malignant glands, adjacent benign glands (nearby the malignant glands), and distal benign glands (located in tissue free of malignant pathology removed from any malignant pathology) as shown in Table 4. The mutations of Table 4 are also provided in SEQ ID No: 102 which lists the substitutions, SEQ ID NOs: 103 to 109 which lists the deletions, and SEQ ID No: 110 to 138 which lists the insertions. Polymorphisms and mutation positions were determined by comparison to the Revised Cambridge Reference Sequence (2001), however the historical numbering has been maintained such that the deletion at position 3106 is denoted as a gap and the rare polymorphism 750A has been retained. The numbering of the bases is based on the revised Cambridge Reference Sequence having a total of 16569 base positions. A histogram showing the number of mutations per location of the mitochondrial genome is shown in FIG. 1. As can be seen in FIG. 1, the mutations were found throughout the mtDNA genome and in all diseased prostates. However, certain “hot spots” were also apparent, for example in the D-loop region and the 16s region. These data sets imply that the designation of malignant or benign tissue, as made by a qualified pathologist using routine histological methods and grading standards, does not identify early disease progression. This strongly suggests that malignant transformation begins at the cellular level before the morphological characteristics of a cell are altered. Importantly, the mutation patterns are completely inconsistent for matched prostate tissue from an individual patient, or in comparison to another patient, perhaps indicating possible tissue sites where clonal expansion of malignant cells may occur. Moreover, separate needle biopsies with the same Gleason score, from the same individual almost always demonstrate alternative mtDNA mutation patterns. This indicates that total mutation load rather than specific mutation sites may be more representative of the disease and progression of the disease.

Since this data was gathered from individuals with known prostate cancer, and in the prostatectomy group with known advanced staging, it is likely that histologically benign tissue has undergone some intracellular transformation(s) associated with neoplasia and possible progression towards malignancy. Benign tissue harboring mtDNA mutations serve as a “biosensor” which can be monitored for increasing mutations indicative of the rate of disease progression. This rate may also indicate tumour aggression. Moreover, the effectiveness of a specific therapy could also be monitored based on the change in this mutational pattern.

This technique may be used as a confirmatory test for benign needle biopsies. Currently when a patient has a needle biopsy performed on the prostate and the tissue looks histologically benign he is sent home and is usually scheduled for follow-up needle biopsies in six months. Use of the above method would examine the already taken needle biopsy tissue and either confirm that the tissue is benign on the molecular level as well, or find evidence that there is in fact a malignancy in the prostate that was geographically missed by the needle biopsy technique, or that the tissue is pre-neoplastic or neoplastic at the molecular level. This could potentially save a lot of people from undergoing multiple surgeries or allow for early preventative treatment.

TABLE 4*
Base Mutations: Observed mutations of homoplasmic to homoplasmic,
homoplasmic to heteroplasmic, and heteroplasmic to homoplasmic

| | BP | | | | | BP | | | |
|---|---|---|---|---|---|---|---|---|---|
| | eliminating | | | | | eliminating | | | |
| BP | position | | | | BP | position | | | |
| Historical | gap at | Homo- | Homo- | Hetero- | Historical | gap at | Homo- | Homo- | Hetero- |
| Numbering** | 3106 | Homo | Hetero | Homo | Numbering** | 3106 | Homo | Hetero | Homo |
| 10 | 10 | T-C | T-T/C | | 200 | 200 | A-G | A-A/G | |
| 31 | 31 | | C-C/T | | 204 | 204 | T-C | T-T/C | |
| | | | | | | | | C-C/T | |
| 41 | 41 | C-T | C-C/T | | 205 | 205 | | A-A/G | |
| 55 | 55 | C-T | | | 207 | 207 | | A-A/G | |
| 57 | 57 | A-T | | | | | G-A | G-G/A | |
| 61 | 61 | | C-C/T | | 208 | 208 | | T-T/C | |
| 64 | 64 | C-T | | | 214 | 214 | | A-A/G | |
| 72 | 72 | C-T | C-C/T | | 217 | 217 | C-T | | |
| | | T-C | T-T/C | | 222 | 222 | C-T | | |
| 73 | 73 | A-G | A-A/G | | 225 | 225 | A-G | A-A/G | |
| 81.1 | 81.1 | INS T | | | 226 | 226 | C-T | C-C/T | |
| 93 | 93 | | G-G/A | | 228 | 228 | A-G | A-A/G | |
| 94 | 94 | | A-A/G | | | | | G-G/A | |
| 104 | 104 | | C-C/T | | 229 | 229 | G-T | | |
| 113 | 113 | | C-C/T | | 234 | 234 | | A-A/G | |
| 119 | 119 | | C-C/T | | 235 | 235 | A-G | | |
| 128 | 128 | C-T | | | | | | G-G/A | |
| 146 | 146 | C-T | C-C/T | | 239 | 239 | T-C | | |
| | | T-C | T-T/C | | 247 | 247 | | G-G/A | |
| 150 | 150 | C-T | C-C/T | | 248 | 248 | DEL A | | |
| | | T-C | T-T/C | | 262 | 262 | | C-C/T | |
| 152 | 152 | C-T | C-C/T | | 263 | 263 | | G-G/T | |
| | | T-C | T-T/C | | | | A-G | A-A/G | |
| 153 | 153 | A-G | A-A/G | | 264 | 264 | T-C | | |
| | | G-A | G-G/A | | 277 | 277 | | C-C/T | |
| 170 | 170 | | C-C/T | | 280 | 280 | | C-C/T | |
| 182 | 182 | C-T | C-C/T | | 295 | 295 | T-C | T-T/C | |
| 185 | 185 | G-A | G-G/A | | 297 | 297 | G-A | G-G/A | |
| | | | A-A/G | | 303.1 | 303.1 | INS C | | |
| | | | G-G/T | | 303.2 | 303.2 | INS C | | |
| 188 | 188 | A-G | | | 305 | 305 | | C-C/T | |
| | | G-A | G-G/A | | 309 | 309 | | T-T/C | |
| 189 | 189 | A-G | A-A/G | | | | | C-C/T | |
| | | G-A | G-G/A | | 309 | 309 | DEL C | | |
| 192 | 192 | T-C | | | 309.1 | 309.1 | INS C | | |
| 194 | 194 | T-C | T-T/C | | 309.2 | 309.2 | INS C | | |
| | | C-T | | | 309.3 | 309.3 | INS C | | |
| 195 | 195 | C-T | C-C/T | | 310 | 310 | DEL C | T-T/C | T/C-T |
| | | | T-T/C | | | | DEL T | C-C/T | |
| 196 | 196 | T-C | | | 311 | 311 | | C-C/T | |
| 198 | 198 | | C-C/T | | 311.1 | 311.1 | INS C | | |
| 199 | 199 | | T-T/C | | 312 | 312 | | C-C/T | |
| | | | C-C/T | | 313 | 313 | | C-C/T | |
| 200 | 200 | G-A | G-G/A | | | | | | |
| 315.1 | 315.1 | INS C | | | 1719 | 1719 | | A-A/G | A/G-A |
| 315.2 | 315.2 | INS C | | | 1761 | 1761 | | | A/T-A |
| 323 | 323 | | G-G/A | | 1766 | 1766 | | | C/T-T |
| 325 | 325 | C-T | C-C/T | | 1811 | 1811 | G-A | G-G/A | |
| 329 | 329 | G-T | | | 1842 | 1842 | | | A/G-A |
| 394 | 394 | | C-C/T | | 1883 | 1883 | | | A/G-G |
| 416.1 | 416.1 | INS G/A | | | 1888 | 1888 | G-A | G-G/A | A/G-G |
| 419 | 419 | | A-A/G | | | | | | A/G-A |
| 456 | 456 | C-T | | | 2005 | 2005 | | | C/T-C |
| | | | T-T/C | | 2056 | 2056 | | | A/G-G |
| 462 | 462 | T-C | | | 2068.1 | 2068.1 | INS A | | |
| 465 | 465 | | T-T/C | | 2075 | 2075 | | | T/G-T |
| 468 | 468 | | C-C/T | | 2257 | 2257 | | | C/T-C |
| 477 | 477 | C-T | | | 2258 | 2258 | | | A/G-A |
| 481 | 481 | C-T | C-C/T | | 2259 | 2259 | T-C | | |
| 482 | 482 | C-T | | | 2261 | 2261 | | C-C/T | |
| 489 | 489 | C-T | C-C/T | | 2280 | 2280 | | | C/T-C |
| 497 | 497 | T-C | T-T/C | | 2351 | 2351 | | | C/T-T |
| 499 | 499 | A-G | | | 2352 | 2352 | | | C/T-T |
| 501 | 501 | | C-C/T | | 2357 | 2357 | | | C/T-C |
| 505 | 505 | | C-C/T | | 2359 | 2359 | | | C/T-C |
| 506 | 506 | | | C/T-C | 2389 | 2389 | | | C/T-C |
| 508 | 508 | G-A | | | 2596 | 2596 | | G-G/A | |
| 513 | 513 | A-G | | | 2627 | 2627 | | G-G/A | |
| 513 | 513 | DEL A | | | 2657 | 2657 | C-T | | |
| 514.1 | 514.1 | INS C | | | 2683 | 2683 | | C-C/T | |
| 515 | 515 | | A-A/G | | 2689 | 2689 | | C-C/T | |
| 515 | 515 | DEL A | | | 2706 | 2706 | A-G | A-A/G | |
| 515.1 | 515.1 | INS A | | | 2761 | 2761 | C-T | | |
| 517 | 517 | | T-T/A | | 2857 | 2857 | | C-C/T | |
| 523 | 523 | | A-A/G | | 2885 | 2885 | | C-C/T | |
| 523 | 523 | DEL A | | | 2927 | 2927 | C-T | | |
| 523.1 | 523.1 | INS C | | | 2948 | 2948 | C-T | | |
| 523.2 | 523.2 | INS A | | | 2952 | 2952 | | | C/T-T |
| 523.3 | 523.3 | INS C | | | 3010 | 3010 | A-G | A-A/G | |
| 523.4 | 523.4 | INS A | | | | | | | |
| 533 | 533 | A-G | A-A\G | | 3013 | 3013 | | G-G/A | |
| 536 | 536 | | C-C/T | | 3036 | 3036 | | | A/G-G |
| 567.1 | 567.1 | INS C | | | 3040 | 3040 | | | A/G-G |
| 568.1 | 568.1 | INS C | | | 3046 | 3046 | | | C/T-C |
| 568.2 | 568.2 | INS C | | | | | | | |
| 709 | 709 | | A-A/G | | 3308 | 3307 | | T-T/C | |
| | | | G-G/A | | 3338 | 3337 | | | C/T-T |
| 785 | 785 | | C-C/G | | 3349 | 3348 | | | A/G-A |
| 857 | 857 | G-C | | | 3394 | 3393 | C-T | | |
| 909 | 909 | | G-G/A | | 3398 | 3397 | T-C | | |
| 1189 | 1189 | | C-C/T | | 3469.1 | 3468.1 | INS T | | |
| 1247 | 1247 | | G-G/A | | 3480 | 3479 | G-A | | |
| 1431 | 1431 | | G-G/A | | 3499 | 3498 | | | A/G-A |
| 1693 | 1693 | C-T | | | 3507 | 3506 | C-A | | |
| 1709 | 1709 | | | A/G-G | 3589 | 3588 | | C-C/T | |
| 1719 | 1719 | | | A/G-G | 3594 | 3593 | | C-C/T | |
| 3657 | 3656 | C-T | | | 5663 | 5662 | | C-C/T | |
| 3666 | 3665 | G-A | | | 5677 | 5676 | C-T | | |
| 3688 | 3687 | | G-G/A | | 5882 | 5881 | | C-C/T | |
| 3693 | 3692 | | G-G/A | | 5897 | 5896 | | C-C/T | |
| 3744.1 | 3743.1 | INS T | | | 5984 | 5983 | A-G | A-A/G | |
| 3908 | 3907 | C-T | | | 5985 | 5984 | | A-A/G | |
| 3966 | 3965 | | C-C/T | | 5999 | 5998 | C-T | | |
| 3969 | 3968 | C-T | | | 6009 | 6008 | | C-C/T | |
| 3992 | 3991 | C-T | | | 6028 | 6027 | | | A/G-G |
| 4017 | 4016 | C-T | | | 6037 | 6036 | | G-G/A | |
| 4185 | 4184 | C-T | | | 6041 | 6040 | | C-C/T | |
| 4216 | 4215 | T-C | T-T/C | | 6047 | 6046 | G-A | | |
| 4217 | 4216 | | A-A/G | | 6059 | 6058 | | C-C/T | |
| 4239 | 4238 | C-T | | | 6147 | 6146 | C-T | | |
| 4418 | 4417 | | C-C/T | | 6219 | 6218 | | C-C/T | |
| 4561 | 4560 | C-T | | | 6221 | 6220 | | C-C/T | |
| 4569 | 4568 | | G-G/A | | 6224 | 6223 | | C-C/T | |
| 4580 | 4579 | | A-A/G | | 6307 | 6306 | | A-A/G | |
| 4591 | 4590 | | T-T/C | | 6314 | 6313 | | C-C/T | |
| 4646 | 4645 | C-T | | | 6382 | 6381 | G-A | | |
| 4655 | 4654 | | A-A/G | | 6548 | 6547 | | C-C/T | |
| 4703 | 4702 | | C-C/T | | 6553 | 6552 | | C-C/T | |
| 4716 | 4715 | | C-C/T | | 6557 | 6556 | | C-C/T | |
| 4722 | 4721 | | A-A/G | | 6579 | 6578 | G-A | | |
| 4733 | 4732 | | C-C/T | | 6643 | 6642 | | T-T/C | |
| 4735 | 4734 | | C-C/T | | 6667 | 6666 | | C-C/T | |
| 4787 | 4786 | G-A | G-G/A | | 6686 | 6685 | T-C | | |
| 4826 | 4825 | | C-C/T | | 6691 | 6690 | G-A | | |
| 4864 | 4863 | | C-C/T | | 6776 | 6775 | C-T | C-C/T | |
| 4892 | 4891 | | C-C/T | | | | T-C | T-T/C | |
| 4917 | 4916 | A-G | A-A/G | | 6827 | 6826 | | T-T/C | |
| | | | G-G/A | | 6912 | 6911 | | G-G/A | |
| 4951 | 4950 | | C-C/T | | 6917.1 | 6916.1 | INS T | | |
| 5036 | 5035 | | A-A/G | | 6953 | 6952 | G-A | | |
| 5046 | 5045 | | G-G/A | | 6989 | 6988 | | A-A/G | |
| 5102 | 5101 | | A-A/G | | 7007 | 7006 | C-T | | |
| 5147 | 5146 | G-A | G-G/A | | 7013 | 7012 | | G-G/A | |
| | | | A-A/G | | 7028 | 7027 | C-T | C-C/T | |
| 5174 | 5173 | | C-C/T | | | | | T-T/C | |
| 5198 | 5197 | | G-G/A | | 7055 | 7054 | | A-A/G | |
| 5213 | 5212 | | C-C/T | | 7059 | 7058 | | G-G/A | |
| 5300 | 5299 | | C-C/T | | 7146 | 7145 | | A-A/G | |
| 5312 | 5311 | | C-C/T | | 7159 | 7158 | | T-T/C | |
| 5371 | 5370 | | C-C/T | | 7184 | 7183 | G-A | G-G/A | |
| 5424 | 5423 | | C-C/T | | 7256 | 7255 | | C-C/T | |
| 5440 | 5439 | | C-C/T | | 7309 | 7308 | | T-T/C | |
| 5456 | 5455 | | C-C/T | | 7389 | 7388 | | T-T/C | |
| 5593 | 5592 | | T-T/C | | 7406.1 | 7405.1 | INS C | | |
| 5633 | 5632 | | T-T/C | | 7407 | 7406 | T-C | T-T/C | |
| 5650 | 5649 | | G-G/A | | 7412 | 7411 | | | (C/T)/(C/T) |
| 5655 | 5654 | | T-C/T | | 7476 | 7475 | | T-T/C | |
| 5656 | 5655 | A-G | A-A/G | | 7521 | 7520 | | G-G/A | |
| 7756 | 7755 | | C-C/T | | 10295 | 10294 | | G-G/A | |
| 7763 | 7762 | | G-G/A | | 10345 | 10344 | | T-T/C | |
| 7768 | 7767 | G-A | A-A/G | | 10355 | 10354 | | C-C/T | |
| 7815 | 7814 | | C-C/T | | 10439 | 10438 | | C-C/T | |
| 7867 | 7866 | | C-C/T | | 10455 | 10454 | | G-G/A | |
| 7897 | 7896 | G-A | | | 10463 | 10462 | T-C | T-T/C | |
| 8027 | 8026 | G-A | | | | | | | |
| 8065 | 8064 | | | G/A-G | 10550 | 10549 | | G-G/A | |
| 8117 | 8116 | | C-C/T | | 10679 | 10678 | G-A | | |
| 8133 | 8132 | | C-C/T | | 10685 | 10684 | | A-A/G | |
| 8248 | 8247 | | A-A/G | | 10688 | 10687 | | G-G/A | |
| 8270 | 8269 | C-T | C-C/T | | 10754 | 10753 | A-C | | |
| 8426.1 | 8425.1 | INS G | | | 10810 | 10809 | T-C | T-T/C | |
| 8468 | 8467 | | C-C/T | | 10819 | 10818 | | G-G/A | |
| 8616 | 8615 | | A-A/G | | 10873 | 10872 | | C-C/T | |
| 8655 | 8654 | | C-C/T | | | | T-C | T-T/C | |
| 8697 | 8696 | G-A | G-G/A | | 10882 | 10881 | C-T | | |
| 8701 | 8700 | | A-A/G | | 10885 | 10884 | C-T | | |
| 8718 | 8717 | A-G | | | 10944 | 10943 | | C-C/T | |
| 8818 | 8817 | | T-T/C | | 10956 | 10955 | | X-C/T | |
| 8893 | 8892 | A-T | | | 10972 | 10971 | G-A | G-G/A | |
| 8903 | 8902 | | C-C/T | | 10975 | 10974 | | C-C/T | |
| 9055 | 9054 | A-G | | | 10978 | 10977 | | A-A/G | |
| 9093 | 9092 | | G-G/A | | 9667 | 9666 | G-A | | |
| 9132 | 9131 | A-G | | | 9696 | 9695 | | C-C/T | |
| 9163 | 9162 | A-G | A-A/G | | 9698 | 9697 | C-T | C-C/T | |
| 9313 | 9312 | | A-A/G | | 9716 | 9715 | C-T | | |
| | | | A-A/C | | 9767 | 9766 | C-T | | |
| 9327 | 9326 | | A-A/G | | 9778 | 9777 | | G-G/A | |
| 9352 | 9351 | | C-C/T | | 9899 | 9898 | C-T | | |
| 9405 | 9404 | | T-T/C | | 10143 | 10142 | A-G | | |
| 9413 | 9412 | | T-T/C | | 10295 | 10294 | | G-G/A | |
| 9419 | 9418 | | C-C/T | | 10345 | 10344 | | T-T/C | |
| 9445 | 9444 | | G-G/A | | 10355 | 10354 | | C-C/T | |
| 9477 | 9476 | G-A | G-G/A | | 10439 | 10438 | | C-C/T | |
| 9502 | 9501 | G-A | | | 10455 | 10454 | | G-G/A | |
| 9540 | 9539 | | C-C/T | | 10463 | 10462 | T-C | T-T/C | |
| 9548 | 9547 | A-G | | | | | | | |
| 9554 | 9553 | | G-G/A | | 10550 | 10549 | | G-G/A | |
| 9559 | 9558 | | C-C/T | | 10679 | 10678 | G-A | | |
| 9564 | 9563 | | G-G/A | | 10685 | 10684 | | A-A/G | |
| 9574 | 9573 | | C-C/T | | 10688 | 10687 | | G-G/A | |
| 9591 | 9590 | | G-G/C | | 10754 | 10753 | A-C | | |
| 9628 | 9627 | | G-G/A | | 10810 | 10809 | T-C | T-T/C | |
| 9667 | 9666 | G-A | | | 10819 | 10818 | | G-G/A | |
| 9696 | 9695 | | C-C/T | | 10873 | 10872 | | C-C/T | |
| 9698 | 9697 | C-T | C-C/T | | | | T-C | T-T/C | |
| 9716 | 9715 | C-T | | | 10882 | 10881 | C-T | | |
| 9767 | 9766 | C-T | | | 10885 | 10884 | C-T | | |
| 9778 | 9777 | | G-G/A | | 10944 | 10943 | | C-C/T | |
| 9899 | 9898 | C-T | | | 10956 | 10955 | | X-C/T | |
| 10143 | 10142 | A-G | | | 10972 | 10971 | G-A | G-G/A | |
| 10975 | 10974 | | C-C/T | | 13398 | 13397 | | A-A/G | |
| 10978 | 10977 | | A-A/G | | 13431 | 13430 | | C-C/T | |
| 11001 | 11000 | | A-A/G | | 13436 | 13435 | | C-C/T | |
| 11013 | 11012 | | X-C/T | | 13468 | 13467 | | C-C/T | |
| 11024 | 11023 | | T-T/C | | 13476 | 13475 | | A-A/G | |
| | | | C-C/T | | 13484 | 13483 | | T-T/C | |
| 11069 | 11068 | | A-A/G | | 13487 | 13486 | | C-C/T | |
| 11084 | 11083 | | A-A/G | | 13506 | 13505 | | C-C/T | |
| 11113 | 11112 | T-C | T-T/C | | 13530 | 13529 | | C-C/T | |
| 11177 | 11176 | | C-C/T | | 13536 | 13535 | | C-C/T | |
| 11180 | 11179 | | G-G/T | | 13563 | 13562 | | A-A/G | |
| 11195 | 11194 | | G-G/A | | 13573 | 13572 | | C-C/T | |
| 11217 | 11216 | | C-C/T | | 13579 | 13578 | G-A | | |
| 11251 | 11250 | A-G | A-A/G | | 13609 | 13608 | | C-C/T | |
| | | G-A | G-G/A | | | | | T-T/C | |
| 11299 | 11298 | C-T | C-C/T | | 13617 | 13616 | C-T | C-C/T | |
| 11332 | 11331 | | T-T/C | | | | T-C | T-T/C | |
| 11337 | 11336 | | G-A/G | | 13634 | 13633 | | G-G/A | |
| 11351 | 11350 | | G-A/G | | 13650 | 13649 | | C-C/T | |
| 11356 | 11355 | | T-T/C | | 13621 | 13620 | | | T/C-C |
| 11377 | 11376 | | A-A/G | | 13631 | 13630 | | C-C/T | |
| 11420 | 11419 | | G-G/A | | 13637 | 13636 | | G-G/A | |
| 11647 | 11646 | C-T | | | 13638 | 13637 | | A-A/G | |
| 11719 | 11718 | G-A | G-G/A | | 13651 | 13650 | | A-A/G | |
| 11812 | 11811 | A-G | A-A/G | | 13655 | 13654 | | T-T/C | |
| 11852 | 11851 | | G-G/A | | 13674 | 13673 | C-T | | |
| | | | | | 13674 | 13673 | DEL C | | |
| 11857 | 11856 | | C-C/T | | 13680 | 13679 | | T-T/C | |
| 11864 | 11863 | C-T | C-C/T | | 13687 | 13686 | | C-C/T | |
| 11881 | 11880 | | C-C/T | | 13707 | 13706 | | G-G/A | |
| 11907 | 11906 | | T-T/C | | 13708 | 13707 | | A-A/G | |
| 11914 | 11913 | | G-G/A | | | | | G-G/A | |
| 12012 | 12011 | | C-C/T | | 13711 | 13710 | | G-G/A | |
| 12013 | 12012 | | | A/G-A | 13712 | 13711 | | C-C/T | |
| 12308 | 12307 | | G-G/A | | 13725 | 13724 | | C-C/T | |
| 12372 | 12371 | | G-G/A | | 13731 | 13730 | | A-A/G | |
| | | | A-A/G | | 13734 | 13733 | | C-C/T | |
| 12492 | 12491 | | T-T/A | | 13743 | 13742 | | T-T/C | |
| 12624 | 12623 | C-T | | | 13748 | 13747 | | A-A/G | |
| 12633 | 12632 | A-C | A-A/C | | 13759 | 13758 | | G-G/A | |
| 12654 | 12653 | | G-G/A | | | | | A-A/G | |
| 12810 | 12809 | | G-G/A | | 13766 | 13765 | | C-C/T | |
| 12959 | 12958 | | C-C/T | | 13788 | 13787 | | C-C/T | |
| 13079 | 13078 | | | A/G-A | 13789 | 13788 | | T-T/C | |
| 13089 | 13088 | T-A | | | 13805 | 13804 | | C-C/T | |
| 13105 | 13104 | | A-A/G | | 13841 | 13840 | | T-T/C | |
| 13111 | 13110 | T-C | | | 13880 | 13879 | | C-C/A | |
| 13212 | 13211 | | C-C/T | | 13888 | 13887 | X-T | X-C/T | |
| 13281 | 13280 | | T-T/C | | 13911 | 13910 | | G-G/A | |
| 13294 | 13293 | | A-A/G | | 13933 | 13932 | | A-A/G | |
| 13359 | 13358 | | G-G/A | | 14025 | 14024 | | C-C/T | |
| 13368 | 13367 | G-A | G-G/A | | 14044 | 14043 | | C-C/T | |
| | | A-G | A-A/G | | 14135 | 14134 | | T-T/A | |
| 14139 | 14138 | | G-G/A | | 15889 | 15888 | | T-T/C | |
| 14167 | 14166 | | T-T/C | | 15904 | 15903 | T-C | T-T/C | |
| 14178 | 14177 | | T-T/C | | | | | C-A/C | |
| 14182 | 14181 | T-C | T-T/C | | 15907 | 15906 | | G-G/A | |
| 14203 | 14202 | | A-A/G | | 15927 | 15926 | A-G | | |
| 14220 | 14219 | X-G | X-G/A | | 15928 | 15927 | A-G | A-A/G | |
| 14233 | 14232 | A-G | A-A/G | | | | G-A | G-G/A | |
| 14281 | 14280 | T-C | T-T/C | | 15998 | 15997 | | | A/T-A |
| | | | C-C/T | | 15999.1 | 15998.1 | INS T | | |
| 14899 | 14898 | | G-G/A | | 16048 | 16047 | | G-G/A | |
| 14903 | 14902 | G-A | X-A/G | | 16051 | 16050 | | A-A/G | |
| 14918 | 14917 | | G-G/A | | | | | G-G/A | |
| 15043 | 15042 | | G-G/A | | 16063 | 16062 | | C-C/T | |
| 15115 | 15114 | | T-T/C | | 16067 | 16066 | | C-C/T | |
| 15162 | 15161 | | C-C/T | | 16069 | 16068 | | C-C/T | |
| 15218 | 15217 | G-A | G-G/A | | 16093 | 16092 | X-C | T-T/C | |
| 15244 | 15243 | | G-G/A | | | | C-T | C-C/T | |
| 15265 | 15264 | | C-C/T | | 16095 | 16094 | C-T | C-C/T | |
| 15286 | 15285 | | C-C/T | | 16111 | 16110 | | T-T/C | |
| 15301 | 15300 | | A-A/G | | 16126 | 16125 | T-C | T-T/C | |
| 15302 | 15301 | | C-C/T | | | | | C-C/T | |
| 15307 | 15306 | | C-C/T | | 16129 | 16128 | G-A | G-G/A | |
| 15323 | 15322 | A-G | A-A/G | | 16134 | 16133 | | C-C/T | |
| | | | G-G/A | | 16148 | 16147 | | C-C/T | |
| 15324 | 15323 | | C-C/T | | 16153 | 16152 | | A-A/G | |
| 15343 | 15342 | X-C | X-C/T | | 16163 | 16162 | | G-G/A | |
| 15355 | 15354 | X-A | X-A/G | | 16172 | 16171 | | C-C/T | |
| 15379 | 15378 | | C-C/T | | 16184 | 16183 | C-T | C-C/T | |
| 15384 | 15383 | X-C | X-C/T | | | | T-C | | |
| 15429 | 15428 | | A-A/G | | 16186 | 16185 | | T-T/C | |
| 15452 | 15451 | C-A | C-C/A | | 16189 | 16188 | T-C | T-T/C | |
| | | A-C | A-A/C | | | | C-T | C-C/T | |
| 15523 | 15522 | | C-C/T | | 16190 | 16189 | T-C | | |
| 15525 | 15524 | | G-G/A | | 16192 | 16191 | | C-C/T | |
| 15526 | 15525 | | C-C/T | | | | T-C | T-T/C | |
| 15527 | 15526 | | C-C/T | | 16209 | 16208 | | C-C/T | |
| 15557 | 15556 | | G-G/A | | | | | T-T/C | |
| 15587 | 15586 | | C-C/T | | 16223 | 16222 | | T-T/C | |
| 15607 | 15606 | A-G | A-A/G | | | | C-T | | |
| | | G-A | G-G/A | | 16224 | 16223 | C-T | C-C/T | |
| 15670 | 15669 | | C-C/T | | | | | T-T/C | |
| 15693 | 15692 | C-T | C-C/T | | 16225 | 16224 | C-T | | |
| 15698 | 15697 | | C-C/T | | 16235 | 16234 | | A-A/G | |
| 15704 | 15703 | | C-C/A | | 16239 | 16238 | C-T | C-C/T | |
| 15708 | 15707 | | G-G/T | | | | X-C | | |
| 15762 | 15761 | | G-G/A | | 16247 | 16246 | | G-G/A | |
| 15812 | 15811 | | A-A/G | | 16256 | 16255 | | T-T/C | |
| 15826.1 | 15825.1 | INS G | | | | | | C-C/T | |
| 15834 | 15833 | | T-T/C | | 16270 | 16269 | C-T | C-C/T | |
| 15865 | 15864 | | A-A/G | | 16292 | 16291 | | C-C/T | |
| 15884 | 15883 | C-G | G-C/G | | 16294 | 16293 | C-T | C-C/T | |
| | | | A-A/G | | 16296 | 16295 | C-T | C-C/T | |
| 16270 | 16269 | T-C | T-T/C | | 16544 | 16543 | | T-T/C | |
| | | | C-C/T | | | | | | |
| 16278 | 16277 | T-C | | | | | | | |
| 16280 | 16279 | | A-A/G | A/G-A | | | | | |
| 16290 | 16289 | | C-C/T | | | | | | |
| | | | T-T/C | | | | | | |
| 16291 | 16290 | | C-C/T | | | | | | |
| 16292 | 16291 | | T-T/C | | | | | | |
| 16293 | 16292 | | A-A/G | | | | | | |
| 16294 | 16293 | | T-T/C | | | | | | |
| | | C-T | C-C/T | | | | | | |
| 16295 | 16294 | | C-C/T | | | | | | |
| 16296 | 16295 | C-T | C-C/T | | | | | | |
| | | | T-T/C | | | | | | |
| 16298 | 16297 | C-T | C-C/T | | | | | | |
| 16303 | 16302 | | G-G/T | | | | | | |
| 16304 | 16303 | | T-T/C | | | | | | |
| | | C-T | C-C/T | | | | | | |
| 16311 | 16310 | C-T | C-C/T | | | | | | |
| | | T-C | T-T/C | | | | | | |
| 16319 | 16318 | G-A | G-G/A | | | | | | |
| | | | T-T/C | | | | | | |
| | | | A-A/G | | | | | | |
| 16320 | 16319 | | T-T/C | | | | | | |
| 16325 | 16324 | | C-C/T | | | | | | |
| 16344 | 16343 | C-G | | | | | | | |
| 16356 | 16355 | | C-C/T | | | | | | |
| 16342 | 16341 | C-T | C-C/T | | | | | | |
| 16352 | 16351 | | T-T/C | | | | | | |
| 16353 | 16352 | | C-C/T | | | | | | |
| 16354 | 16353 | | T-T/C | | | | | | |
| 16355 | 16354 | | T-T/C | | | | | | |
| 16359.1 | 16358.1 | INS G | | | | | | | |
| 16360 | 16359 | | C-C/T | | | | | | |
| 16362 | 16361 | | C-C/T | | | | | | |
| | | | T-T/C | | | | | | |
| 16370 | 16369 | | G-G/A | | | | | | |
| 16389 | 16388 | | G-G/A | | | | | | |
| 16390 | 16389 | X-G | X-G/A | | | | | | |
| 16398 | 16397 | A-G | | G/A-G | | | | | |
| 16399 | 16398 | G-A | | G/A-A | | | | | |
| 16429 | 16428 | | X-T/C | | | | | | |
| 16465 | 16464 | C-T | C-C/T | | | | | | |
| | | T-C | T-T/C | | | | | | |
| 16475 | 16474 | | T-T/C | | | | | | |
| 16514 | 16513 | | C-C/T | | | | | | |
| 16519 | 16518 | T-C | T-T/C | | | | | | |
| | | C-T | C-C/T | | | | | | |
| 16526 | 16525 | A-G | | | | | | | |
| | | | G-G/A | | | | | | |
| 16527 | 16526 | | C-C/T | | | | | | |
| 16537 | 16536 | T-C | T-T/C | | | | | | |

*Sequences found in SEQ ID NO: 102 to 138 of Sequence Listing
*The first nucleotide represents the normal nucleotide, followed by the mutated nucleotide; separated by an “-”.
*When no blood is present, it is denoted as an “X”.
**Historical numbering wherein deletion at BP 3106 is denoted as a gap and the rare polymorphism 750A has been retained

TABLE 5

| SEQ | | Length | |
|---|---|---|---|
| ID | | (# | |
| NO: | Primer | bases) | 5′-3′ |

D-Loop Primers (used for formalin fixed tissue
and blood for needle biopsies)

| | | | |
|---|---|---|---|
| 19 | 15971f | 20 | TTAACTCCACCATTAGCACC |
| 20 | 15f | 20 | CACCCTATTAACCACTCACG |
| 21 | 16211f | 22 | CAGCAATCAACCCTCAACTATC |
| 22 | 16410r | 19 | AGGATGGTGGTCAAGGGAC |
| 23 | 389r | 20 | CCTAACACCAGCCTAACCAG |
| 24 | 420r | 18 | GTGCATACCGCCAAAAGA |
| 25 | 711r | 21 | AACGGGGATGCTTGCATGTGT |

Formalin Fixed Tissue Primers (used for 31
prostatectomies)

| | | | |
|---|---|---|---|
| 26 | 649f | 21 | TAGGTTTGGTCCTAGCCTTTC |
| 27 | 1051f | 26 | ACAATAGCTAAGACCCAAACTGGGAT |
| 28 | 1247r | 22 | CAAGAGGTGGTGAGGTTGATCG |
| 29 | 8959r | 22 | CGATAATAACTAGTATGGGGAT |
| 30 | 8814f | 22 | CCAACTATCTATAAACCTAGCC |
| 31 | 9247f | 19 | GCCCATGACCCCTAACAGG |
| 32 | 9868r | 21 | CGGATGAAGCAGATAGTGAGG |
| 33 | 9711f | 22 | CTGGGTCTCTATTTTACCCTCC |
| 34 | 10663f | 18 | TCTTTGCCGCCTGCGAAG |
| 35 | 10766r | 22 | TTAGCATTGGAGTAGGTTTAGG |
| 36 | 11813r | 26 | GTAGAGTTTGAAGTCCTTGAGAGAGG |
| 37 | 11629f | 23 | AATCAGCCACATAGCCCTCGTAG |
| 38 | 12709r | 28 | GGAAGATGAGTAGATATTTGAAGAACTG |
| 39 | 12528f | 22 | GAACTGACACTGAGCCACAACC |
| 40 | 13516r | 23 | GGTCTTTGGAGTAGAAACCTGTG |
| 41 | 13239f | 23 | CGTAGCCTTCTCCACTTCAAGTC |
| 42 | 15351r | 23 | TCGTGCAAGAATAGGAGGTGGAG |
| 43 | 15144f | 25 | TCCCGTGAGGCCAAATATCATTCTG |
| 44 | 6145r | 24 | CAGTTGCCAAAGCCTCCGATTATG |
| 45 | 5867f | 25 | CAATGCTTCACTCAGCCATTTTACC |
| 46 | 13957r | 22 | CTAGATAGGGGATTGTGCGGTG |
| 47 | 13838f | 23 | CCCTAGACCTCAACTACCTAACC |
| 48 | 15026r | 21 | GGCAGATAAAGAATATTGAGG |
| 49 | 14937f | 22 | CATCAATCGCCCACATCACTCG |
| 50 | 1938f | 24 | AGAGCACACCCGTCTATGTAGCAA |
| 51 | 2084r | 26 | TACAAGGGGATTTAGAGGGTTCTGTG |
| 52 | 2973f | 24 | TAGGGTTTACGACCTCGATGTTGG |
| 53 | 3101r | 24 | TAGAAACCGACCTGGATTACTCCG |
| 54 | 3728f | 23 | CATATGAAGTCACCCTAGCCATC |
| 55 | 3893r | 23 | GTTCGGTTGGTCTCTGCTAGTGT |
| 56 | 4888f | 27 | CAATCATATACCAAATCTCTCCCTCAC |
| 57 | 5035r | 25 | CATCCTATGTGGGTAATTGAGGAGT |
| 58 | 5981f | 23 | TGGAGTCCTAGGCACAGCTCTAA |
| 59 | 6154r | 24 | GGAACTAGTCAGTTGCCAAAGCCT |
| 60 | 6911f | 24 | TGCAGTGCTCTGAGCCCTAGGATT |
| 61 | 7082r | 26 | GAAGCCTCCTATGATGGCAAATACAG |
| 62 | 7829f | 25 | CGCATCCTTTACATAACAGACGAGG |
| 63 | 8029r | 24 | GGCTTCAATCGGGAGTACTACTCG |

Blood Primers (prostatectomy)

| | | | |
|---|---|---|---|
| 64 | 16485f | 24 | GAACTGTATCCGACATCTGGTTCC |
| 65 | 919r | 22 | TTGGGTTAATCGTGTGACCGCG |
| 66 | 1644r | 26 | CTCCTAAGTGTAAGTTGGGTGCTTTG |
| 67 | 615f | 24 | ATGTTTAGACGGGCTCACATCACC |
| 68 | 1488f | 24 | CGTCACCCTCCTCAAGTATACTTC |
| 69 | 2612r | 28 | GGAACAAGTGATTATGCTACCTTTGCAC |
| 70 | 2417f | 23 | CACTGTCAACCCAACACAGGCAT |
| 71 | 3641r | 23 | GCTAGGCTAGAGGTGGCTAGAAT |
| 72 | 3230f | 23 | GTTAAGATGGCAGAGCCCGGTAA |
| 73 | 4417r | 26 | TTTAGCTGACCTTACTTTAGGATGGG |
| 74 | 4337f | 24 | ATGAGAATCGAACCCATCCCTGAG |
| 75 | 5551r | 24 | GGCTTTGAAGGCTCTTGGTCTGTA |
| 76 | 6418f | 23 | AACCCCCTGCCATAACCCAATAC |
| 77 | 7554r | 33 | CTTTGACAAAGTTATGAAATGGTTTTTCTAATA |
| 78 | 7400f | 22 | CCCACCCTACCACACATTCGAA |
| 79 | 8441r | 26 | GTTGGGTGATGAGGAATAGTGTAAGG |
| 80 | 8346f | 26 | CAACACCTCTTTACAGTGAAATGCCC |
| 81 | 9413r | 24 | GCCTTGGTATGTGCTTTCTCGTGT |
| 82 | 10285r | 21 | GGTAGGGGTAAAAGGAGGGCA |
| 83 | 9273f | 21 | TCAGCCCTCCTAATGACCTCC |
| 84 | 10198f | 19 | CCCGCGTCCCTTTCTCCAT |
| 85 | 11408r | 25 | GGAGTCATAAGTGGAGTCCGTAAAG |
| 86 | 11210f | 24 | TTCTACACCCTAGTAGGCTCCCTT |
| 87 | 12231r | 26 | GTTAGCAGTTCTTGTGAGCTTTCTCG |
| 88 | 12096f | 22 | TCCTATCCCTCAACCCCGACAT |
| 89 | 13098r | 26 | CAACTATAGTGCTTGAGTGGAGTAGG |
| 90 | 12881f | 26 | CATCCTCGCCTTAGCATGATTTATCC |
| 91 | 13851r | 24 | GTTGAGGTCTAGGGCTGTTAGAAG |
| 92 | 14738f | 24 | AGAACACCAATGACCCCAATACGC |
| 93 | 15731r | 28 | CTAGGAGTCAATAAAGTGATTGGCTTAG |
| 94 | 15347f | 23 | CACGAAACGGGATCAAACAACCC |
| 95 | 16000r | 24 | CTTAGCTTTGGGTGCTAATGGTGG |
| 96 | 5544f | 21 | CACGCTACTCCTACCTATCTC |
| 97 | 6482r | 20 | GACTGCTGTGATTAGGACGG |
| 98 | 13354f | 23 | TTTATGTGCTCCGGGTCCATCAT |
| 99 | 14458r | 22 | GATGGCTATTGAGGAGTATCCT |
| 100 | 14399f | 21 | ACACTCACCAAGACCTCAACC |
| 101 | 15593r | 23 | ATCGGAGAATTGTGTAGGCGAAT |

### REFERENCES

Alonso, A. C Alves, M. P. Suarez-Mier, C Albarran, L Pereira, L Fernandez de Simon, P. Martin, O Garcia, L Gusmao, M Sancho, A Amorim 2005. J Clin Pathology 58: 83-86.
Anderson S, et al., Nature 290:457-464, 1981
Andrews R M, et al., Nature Genetics 23(2):147, 1999
Barringer et al., Gene, 89:117 1990
Bassam B J, Caetano-Anolles P M, Gresshoff P M., Anal. Biochem. 196: 80-83, 1991
Bemeburg M, et al., J. Biol. Chem. 274(22):15345-15349, 1999
Berthon P, Valeri A, Cohen-Akeninc A, Drelon E, Paiss T, Wohr G, Latil A et al., Am. J. Hum. Genet., 62: 1416-1424, 1998
Birch-Machin M A, et al., Methods in Toxicology, Volume 2, 51-69, 1993
Birch-Machin M A, et al., J. Invest. Dermatol., 110:149-152 1998
Birch-Machin M A, Online Conference Report (Sunburnt DNA), International Congress of Biochemistry and Molecular Biology, New Scientist, 2000(a)
Birch-Machin M A, Taylor R W, Cochran B, Ackrell B A C, Tumbull D M. Ann Neurol 48: 330-335, 2000(b)
Birch-Machin M A, Clin. Exp. Dermatol, 25(2), 141-146, 2000 (c)
Birch-Machin M A and Krishnan K. Mitochondrion, 1, p45 (2001).
Birch-Machin M A, Lindsey J. Lusher M and Krishnan K. Mitochondrion, 1 Suppl. 1, S30 (2001).
Bogliolo, M, et al., Mutagenesis, 14: 77-82, 1999
Brierley E J, Johnson M A, Lightowlers R N, James O, Tumbull D M., Ann Neurol 43(2):217-223, 1998
Brockington, et al., Nature Genet 4:67-71, 1993
Brown, M. D., et al., Am J. Humn Genet, 60: 381-387, 1997
Brumley, R. L. Jr. and Smith, L. M., 1991, Rapid DNA sequencing by horizontal ultrathin gel electrophoresis, Nucleic Acids Res. 19:4121-4126 Nucleic Acids Res. 19: 4121-4126
Buttyan R, Sawczuk I S, Benson M C, Siegal J D, Olsson C A., Prostate 11:327-337, 1987
Byrne E., Curr Opin Reumatol 4(6):784-793, 1992
Caims P, Okami K, Halachmi S, Halachmi N, Esteller M, Herman J G, Jen J et al., Cancer Res 57:4997-5000, 1997
Carew J. S. and Huang P. Molecular Cancer http://www.molecular-cancer.com (2002)
Chee, M. et al Science 274; 610-614, 1996
Chen J. Z. et al. Cancer Research (62): 6470-6474 (2002).
Chen J. Z. et al. Carcinogenesis (Vol 24) No. 9 1481-1487 (2003)
Chinnery P F, Howel N, Turnbull D M. J. Med. Genet.; 36: 425436, 1999
Chinnery P F and Turnbull D M., Lancet 354 (supplement 1): 17-21, 1999
Chinnery P F and Tumbull D M., Lancet 354 (supplement I): 17-21, 2000
Chollat-Traquet, C, Tobacco or health: a WHO programme., Eur J Cancer, 28(2-3): 311-315, 1992
Chomyn A, et al., Proc. Natl. Acad. Sci. USA 89(10):4221-4225, 1992
Cohen D, Barton G, The cost to society of smoking cessation., Thorax. 53(2): S3842, 1998
Corral-Debrinski et al., Mutat Res, 275: 169-180, 1991
Cortopassi G. A. and Arnheim, H. Detection of a specific mitochondiral DNA deletion in tissues of older humans, Nucleic Acids Tes. 18, 6927-6933 1990
Cortopassi G, Wang E., Biochim Biophys Acta 1271(1):171-176, 1995
Croteau D L, Stierum R H, Bohr V A, Mutat Res 434(3):137-148, 1999
Current Protocols in Molecular Biology
Davis R M, Boyd G M, Schoenborn C A, “Common courtesy” and the elimination of passive smoking. Results of the 1987 National Health Interview Survey. JAMA 263(16): 2208-10, 1990
Dong J T, Isaacs W B, Rinker-Schaeffer C W, Vukanovic J, Ichikawa T, Isaaca J T, Barrett J C., Science 268:884-886, 1995
Driezen P, Brown K S., Searchable database of questionnaire items from populations surveys of tobacco use in Canada: A summary report to the Ontario Tobacco Research Unit (Toronto, Ontario) 1999
Easton R D, Merriwether A D, Crews D E, and Ferrell R E., Am. J. Hum. Genet. 59:202-212, 1996
Fahn H, Wang L, Hseith R, Chang S, Kao S, Huang M, and Wei Y. American Journal of Respiratory Critical Care Medicine, 154:1141-1145, 1996
Fahn H J, Wang L S, Kao S H, Chang S C, Huang M H, Wei Y H., Am. J. Respir. Cell. Mol. Biol., 19(6): 901-9, 1998
Finegold D., Mitochondrial Disease-Primary Care Physican's Guide. Psy-Ed. Corp D/B/A Exceptional Parents Guide: 12, 1997
Flanagan N, Birch-Machin M A, Rees J L., Hum Mol Genet 9 (17):2531-2537, 2000
Flanagan N, Ray A J, Todd C, Birch-Machin M A and Rees J L. J Invest. Dermatol (2001) 117 (5) 1314-1317
Fliss M S, et al. Science 287: 2017-2019, 2000
Gattermann, N, Berneburg, M, Heinisch, J, Aul, C, Schneider, W., Leukemia 9(10): 1704-10, 1995
Green R, Reed J C., Science 281 (5381):1309-1312, 1998
Guatelli, et al., Proc. Nat. Acad. Sci. U.S.A. 87: 1874 1990
Gulavita, Sunil Dr. Northwestern Ontario Cancer Centre—Personal Communication
Habano S, Nakamura, Sugai T., Oncogene 17 (15):1931-1937, 1998
Harding R M, et al., Am. J. Hum. Genet. 66, 1351-1361, 2000
Harman, D., Proc Nati Acad Sci USA 78(11): 7124-8, 1981
Hattori et al, Age-dependant increase in deleted mitochondrial DNA in the human heart: possible contributory factor to presbycardia, AM. Heart J., 121, 1735-1742, 1991
Hayashi J, Ohta S, Kikuchi A, Takemitsu M, Goto Y, Nonaka 1., Proc Natl Acad Sci USA 88 (23):10614-10618, 1991
Hayashi J, Ohta S, Kikuchi A, Takemitsu M, Goto Y, Nonaka I., Proc Natl Acad Sci USA 88 (23):10614-10618, 1991
Hayward S W, Grossfeld G D, TIsty T D, Cunha G R., Int J Oncol 13:3547, 1998
Healy E, Birch-Machin M A, Rees J L. Chapter 11. The Human Melanocortin 1 Receptor Gene. In the Melanocortin Receptors(Cone R D (ed)). Humana Press Inc. New Jersey, USA, 1999
Healy E, Birch-Machin M A, Rees J L., Lancet 355, 1072-1073, 2000
Hearst N, Hulley S B. Using secondary data, In Designing clinical research: an epidemiological approach. Ed. Hulley, S. and Cummings, S., Baltimore: Williams & Wilkins, pages 53-62, 1988
Hopgood, R., et al, 1992, Strategies for automated sequencing of human mtDNA directly from PCR products, Biotechniques 13:82-92
Hsieh, R H and Wei, Y H, Age-dependent multiple deletions in human muscle mitochondrial DNA, in preparation 1992
Huang G M, Ng W L, Farkas J, He L, Liang H A, Gordon D, Hood R., Genomics 59(2):178-86, 1999
Huoponen, Kirsi, Leber hereditary optic neuropathy: clinical and molecular genetic findings, Neurogenetics (2001) 3: 119-125. Http://www.oml.gov/hgmis/project/budget.html)
Ikebe et al., Increase of deleted mitochondrial DNA in the striatum in Parkinson's disease and senescence, Biochem. Biophys. Res. Commun. 170, 1044-1048, 1990
Innis et al PCR Protocols, A Guide to Methods and Application, Academic Press Inc. San Diego 1990
Kaiserman M J, Chronic Dis Can 18(1): 13-9, 1997
Kalra J, Chaudhary A K, Prasad K, Int. J. Exp. Pathol. 72(1): 1-7, 1991
Katayama et al., Deleted mitochondrial DNA in the skeletal muscle of aged individuals, Biochem. Int., 25, 47-56 1991
Kleinle S, et al., Human Genet. 290: 457-465, 1997
Konishi N, Cho M, Yamamoto K, Hiasa Y. Pathol. Int. 47:735-747, 1997
Krishnan K and Birch-Machin M A. British Journal of Dermatology (2002), 146, 723
Kwoh et al. Proc. Natl. Acad. Sci. U.S.A., 86: 1173 1989
Landis S H, Murray T, Bolden S, Wingo P A. Cancer J. Clin. 49:8-31
Landis, S H, et al., CA Cancer J. Clin. 49: 8-31, 1999
Landegren et al. Science, 241:1077 1988
LeDoux S P, et al. Mutat Res 434(3):149-159, 1999
Lee H C, et al. FEBS Letters 354:79-83, 1994
Lee H C, Lu C Y, Fahn H J, Wei YHu. Federation of European Biochemical Societies, 441:292-296, 1998
Lee H C, et al. Arch. Biochem. Biophys. 362(2): 309-16, 1999
Leonard & Shapira 1997
Lindsey J, Lusher M, Krishnan K J and Birch-Machin M A., British Journal of Dermatology (2001), 144,655
Li Y, et al., In: Oxygen Radicals and the Disease Process, Amsterdam, The Netherlands: Harwood Academic Publishers, 237-277, 1997
Linnane et al., 1990
Liu C S, Kao S H, Wei Y H. Environ. Mol. Mutagen 30(1): 47-55, 1997
Lopez, J. V. et al. (1994) Numt, a recent transfer and tandem amplification of mitochondrial DNA to the nuclear genome of the cat. J. Mol. Evol. 39, 174-190.
Lowes S, Krishnan K, Lindsey J, Lusher M and Brich-Machin M A. British Journal of Dermatology (2002, 146,736
Luckey, J. A., et al, 1993, High speed DNA sequencing by capillary gel electrophoresis, Methods Enzymol. 218:154-172
McCormack, Douglas. Website: http://cormactech.com/dna, 2001
Meibner C, von Wurmb N, Oehmichen M., Int. J. Legal Med. 110: 288-291, 1997
Michikawa Y, Mazzucchelli F, Bresolin N, Scarlato G, Attardi G., Science 286: 774-779, 1999
Miquel J, de Juan E, Sevila I. EXS 62:47-57, 1992
MITOMAP: A human mt genome database (www.gen.emory.edu/mitomap.html)
MITOMAP: A Human Mitochondrial Genome Database. http://www.mitomap.org, 2005.
Mitochondrial Research Society http://www.mitoresearch.org/diseases.html.
Mullis and Faloona Methods Enzymol 155, 335 1987
Nachman M W, Brown W M, Stoneking M, Aquardo C F., Genetics 142:53-963, 1996
National Cancer Institute of Canada, Canadian cancer statistics 2000., National Cancer Institute of Canada., Toronto, Ont. 2000
Naviaux, R K., Mitochondrial Disease-Primary Care Physican's Guide. Psy-Ed. Corp D/B/A Exceptional Parents Guide: 3-10, 1997
Newton, C R and Graham, A., Introduction toBiotecniques Series 1997
Oefner P J, Underhill P A., Current protocols in human genetics 19, 7.10.1-12, 1998
Ozen M, et al, Prostate 36:264-271, 1998
Pang et al, Arch. Biochem. Biophys. 312: 534-538, 1994
Parsons T J, et al., Nature Genet. 15 (4):363-368, 1997
Pascucci B, et al., J. Mol. Biol. 273 (2):417-427, 1997
Penta J S, Johnson F M, Wachsman J T, Copeland W C., Mut. Res. 488, 119-133, 2001
Polyak Y, et al., Nature Genet. 20 (3):291-293, 1998
Ray A J, Rees J L, Birch-Machin M A., J. Invest. Dermatol. 110:692, 1998
Ray A J, Rees J L, Birch-Machin M A., Brit. J. Dermatol. 140:788, 1999
Ray A J, Pickersgill L, Turner R, Nikaido O, Rees J L, Birch-Machin M A., J. Invest. Dermatol 115(4):674-679, 2000
Rees J L, Skin cancer. In: The Genetic Basis of Human Cancer, eds Vogelstein B, Kinzler K. New York: McGraw-Hill, pp527-536, 1998
Rehman I, Quinn A J, Healy E, Rees J L. Lancet 344: 788-789, 1994
Rehman I, Takata M, Wu Y Y, Rees J L. Oncogene 12: 2483-2490, 1996
SAS Enterprise Mining Users Guide, SAS Inc., 2000
Sawyer E, Van Houten B., Mutation Res: 434(3):161-176, 1999
Schurr T G, Ballinger S W, Gan Y, Hodge J A, Merriwether D A, Lawrence D N, Knowler W C, Weiss K M, and Wallace D C., Am. J. Hum. Genet. 46:613-623, 1990
Seidman, M. D. et al., Arch. Otolaryngol Head Neck Surg., 123: 1039-1045, 1997
Seo, Jinwook, Shneiderman, Ben, Interactively Exploring Hierarchical Clustering Results, IEEE Computer, Volume 35, Number 7, pp. 80-86, July 2002. [initial draft (pdf)]
Seo, Jinwook, et al., Interactive Color Mosaic and Dendrogram Displays for Signal/Noise Optimization in Microarray Data Analysis, IEEE International Conference on Multimedia and Expo 2003.
Seo, Jinwook, Shneiderman, Ben, Interactive Exploration of Multidimensional Microarray Data: Scatterplot Ordering, Gene Ontology Browser, and Profile Search, HCIL-2003-25, CS-TR-4486, UMIACS-TR-2003-55.
Seo, Jinwook et al., Interactively optimizing signal-to-noise ratios in expression profiling: project-specific algorithm selection and detection p-value weighting in Affymetrix microarrays, Bioinformatics, Vol. 20, pp. 2534-2544, 2004a.
Seo, Jinwook, Shneiderman, Ben, A Rank-by-Feature Framework for Unsupervised Multidimensional Data Exploration Using Low Dimensional Projections, Proc. IEEE InfoVis 2004b, pp. 65-72.
Seo, Jinwook, Shneiderman, Ben, A Rank-by-Feature Framework for Interactive Exploration of Multidimensional Data, will appear in the journal, Information Visualization, 2005a. (pdf)
Seo, Jinwook, Shneiderman, Ben, Understanding Clusters in Multidimensional Spaces: Mading Meaning by Combining Insights from Coordinated Views of Domain Knowledge, Technical Report, HCIL-2004-03, 2004c.
Seo, Jinwook, Shneiderman, Ben, Knowledge Integration Framework for Information Visualization, will be published in LNCS by Springer-Verlag, Berlin Heidelberg New York, 2005b. (pdf)
Shankey T V, Jin J K, Dougherty S, Flanigan R C, Graham S, Pyle J M., Cytometry 21:30-39, 1995
Shay J W, Werbin H., Mutat. Res: 186: 149, 1987
Sherrat E J, Thomas A W, Alcolado J C., Clin. Sci. 92:225-235, 1997
Shoffner J M, Brown M D, Torroni A, Loft M T, Cabell M F, Mirra S S, Beal M F, Yang C, Gearing M, Salvo R, Watts R L, Juncos J L, Hansen L A, Crain B J, Fayad M, Reckford C L, and Wallace D C., Genomics 17:171-184, 1993
Singh K. K. and Modica-Napolitano J. S. Expert reviews in molecular medicine. http://www.ermm.cbcu.ac.uk (2002)
Smith D G, Malhi R S, Eshleman J, Lorenz J G and Kaestle F A., Am. J. Hum. Genet. 110:271-284, 1999
Smith R, Birch-Machin M A, Rees J L. J. Invest. Dermatol. 111: 101-104, 1998 SpringNet—CE Connection: Screening, Diagnosis: Improving Primary Care Outcomes. Website: http://www.springnet.com/ce/j803a.htm
Stone A C and Stoneking M. Amer. J., Phys. Anthro. 92(4):463-471, 1993
Tamura S, et al. Eur. J. Cancer[A] 35 (2):316-319, 1999
Tanaka, M. et al, 1996, Automated sequencing of mtDNA, Methods Enzymol. 264: 407-421
Taniike, M. et al., BioChem BioPhys Res Comun, 186: 47-53, 1992
Taylor R W, Birch-Machin M A, Bartlett K, Turnbull D M., J Biol Chem, 269, 3523-3528 1994
Tijssen, P. (ed) Laboratory Techniques in Biochemistry and Molecular Biology, Vol. 24: Hybridization with Polynucleotide Probes, Elsevier, N.Y., 1993
Tori et al., Ageing-associated deletions of human diaphragmatic mitochondrial DNA, AM. J. Respir. Cell. Mol. Biol. in press 1992
Valnot, Isabelle, et al., A mitochondrial cytochrome b mutation but no mutations of nuclearly encoded subunits in ubiquinol cytochrome c reductase (complex III) deficiency, Human Genetics (1999) 104: 460-466.
Van De Graff, K M, Fox, S I. Concepts of Human Anatomy and Physiology., Dubuque: W M. C. Brown Publishers, 1995
Van den Bosch B J C, et al., Nucleic Acids Res. 28: 89, 2000
von Wurmb, N, Oehmichen, M, Meissner, C., Mutat Res. 422:247-254, 1998
Wallace D C., Annu Rev Biochem, 61: 1175-1212, 1992
Wallace D C. Proc. Natl. Acad. Sci. USA 91: 8739-8746, 1994
Wald and Wallace, D. C., Mitochondrial Diseases in man and Mouse. Science, 5(283): 1482-1497, 1999
Wald, N J, Hackshaw, A K, Cigarette Smoking: an epidemiological overview. Br Med Bull. 52(1): 3-11, 1996
Wallace et al., Mitochondiral DNA MUtatio Assoicated with Leber's Hereditary Optic Neuropathy, Science, 1427-1429
Walsh P C, Partin A W. Cancer 80:1871-1874, 1997
Ward R H, Frazier B L, Dew-Jager K, Paabo S., Proc. Natl. Acad. Sci. USA 88:8720-8724, 1991 Ward 1993
Wei Y H, Pang C, You B, Lee H. Ann. NY Acad. Sci. 786:82-101, 1996
Wei Y H. Proceedings of the Nat. Sci. Council of the Republic of China April 22(2):5567, 1998
Weinstock M A: In: J J Stem R S, MacKie R M and Weinstock M A, Grob (eds) Epidemiology, Blackwell (UK). pp121-128, 1998
Woodwell D A. National Ambulatory Medical Care Survey: 1997 Summary. Advance data from vital and health statistics; no. 305. Hyattsville, Md.: National Center for Health Statistics. 1999
Wu & Wallace Genomics, 4:560, 1989
Xu J, et al., Nature Genet 20: 175-179, 1998
Yamaguchi K T, et al., Free Radical Res. Commun. 16(3):167-74, 1992
Yeh, J. J., et al., Oncogene Journal, 19: 2060-2066, 2000
Yen et al., Ageing-associated 5 kb deletion in human liver mitochondrial DNA, Biochem., Biophys., Res. Commun., 178, 124-131 1991
Yen et al., Age-dependent 6 kb deletion in human liver mitochondirial DNA, Biochem. Int. 26, 457-468 1992
Zeviani M, et al. Am. J. Hum. Genet. 47:904-914, 1990
Zhang et al., Multiple mitochondiral DNA deletions in an elderly human individual, FEBS Lett, 297, 34-38 1992
Zhang, C., et al., BioChem. BioPhys. Res. Comun., 195:1104-1110, 1993
Zhao, Po et al., In vivo filtering of in vitro MyoD target data: An approach for identification of biologically relevant novel downstream targets of transcription fctors, Comptes Rendus Biologies, Vol. 326, Issues 10-11, October-November 2003, pp 1049-1065.

We claim:

1. Use of a synonymous or non-synonymous mutation in an ND1 nucleic acid molecule of mtDNA, to detect a predisposition to prostate cancer, early detection of prostate cancer, genesis of prostate cancer, presence of prostate cancer, or progression of prostate cancer in a subject having mtDNA.

2. Use of a synonymous or non-synonymous mutation in an ND2 nucleic acid molecule of mtDNA, to detect a predisposition to prostate cancer, early detection of prostate cancer, genesis of prostate cancer, presence of prostate cancer, or progression of prostate cancer in a subject having mtDNA.

3. Use of a synonymous or non-synonymous mutation in a COX1 nucleic acid molecule of mtDNA, to detect a predisposition to prostate cancer, early detection of prostate cancer, genesis of prostate cancer, presence of prostate cancer, or progression of prostate cancer in a subject having mtDNA.

4. Use of a synonymous or non-synonymous mutation in a CytB nucleic acid molecule of mtDNA, to detect a predisposition to prostate cancer, early detection of prostate cancer, genesis of prostate cancer, presence of prostate cancer, or progression of prostate cancer in a subject having mtDNA.

5. The use of any of claims 1 to 4 wherein the mutation is detected in malignant tissue, adjacent benign tissue, or distant benign tissue in a subject having prostate cancer or progressing toward prostate cancer.

6. A method of detecting a mutation associated with prostate cancer in a subject having mtDNA, comprising:
a. providing a biological sample from the subject, wherein the biological sample is chosen from non-involved tissue, distant benign tissue, adjacent benign tissue, atypical tissue, histologically abnormal tissue, and malignant tissue;
b. extracting DNA from the biological sample;
c. detecting the presence of the mutation in ND1, ND2, COX1 or CytB of mtDNA; and
d. determining whether the mutation is associated with normal interpopulation or intrapopulation variations, or whether the mutation is associated with prostate cancer.

7. The method of claim 6 further comprising at least one of
a. determining total mutation load in the mtDNA of the biological sample; and
b. determining the identity of the mutation in the mtDNA of the biological sample.

8. The method of claim 6 where the step of determining whether the mutation is associated with normal interpopulation or intrapopulation variations, or whether the mutation is associated with prostate cancer, comprises at least one of:
(a) comparing the mtDNA of the biological sample to a database, the database containing data of interpopulation and intrapopulation variations, and mutations associated with prostate cancer; and
(b) determining total mutation load of the biological sample.

9. The method of claim 6 for use in a diagnosis, wherein the diagnosis is chosen from predisposition to prostate cancer, early detection of prostate cancer, genesis of prostate cancer, presence of prostate cancer, and progression of prostate cancer.

10. The method of claim 6 wherein the step of detecting the presence of mutations is chosen from:
(a) sequencing the mtDNA;
(b) amplifying mtDNA by PCR;
(c) Southern, Northern, Western and South-Western blot hybridizations;
(d) denaturing HPLC;
(e) hybridization to microarrays, gene chips or biochips;
(f) molecular marker analysis; and
(g) a combination of any of a) through f).

11. The method of claim 10 wherein the mitochondrial DNA which is sequenced comprises the entire mitochondrial genome.

12. The method of any of claims 6 to 11 wherein the mutation is a synonymous or non-synonymous mutation.

13. The method of claim 6 where the mutation is heteroplasmic at any level.

14. An array comprising a plurality of nucleic acid members, and a solid substrate, wherein each of the nucleic acid members is associated with at least one mutation in ND1, ND2, COX1 or CytB of the mitochondrial genome and is indicative of the presence of prostate cancer, and is chosen from mitochondrial DNA, RNA transcribed from mitochondrial DNA, and cDNA, wherein each nucleic acid member has a unique position on said array and is stably associated with the solid substrate.

15. An array comprising a plurality of nucleic acid members, and a solid substrate, wherein each of the nucleic acid members is associated with at least one mutation in ND1, ND2, COX1 or CytB of the mitochondrial genome and is indicative of the progression toward prostate cancer, and is chosen from mitochondrial DNA, RNA transcribed from mitochondrial DNA, and cDNA, wherein each nucleic acid member has a unique position on said array and is stably associated with the solid substrate.

16. A kit for diagnosing prostate cancer comprising a disposable chip, the array of any of claims 14 and 15, means for holding the disposable chip, means for extraction of mitochondrial DNA and means for access to a database of mitochondrial DNA sequences.

17. A kit for determining the progression toward prostate cancer or early detection of prostate cancer comprising a disposable chip, the array of any of claims 14 and 15, means for holding the disposable chip, means for extraction of mitochondrial DNA and means for access to a database of mitochondrial DNA sequences.

18. A database containing mitochondrial DNA sequences for ND1, ND2, COX1 and CytB, the sequences are chosen from normal control sequences associated with non-disease states, sequences associated with interpopulation variations, sequences associated with intrapopulation variations, and sequences associated with prostate cancer.

19. A method of monitoring a person for the progression toward prostate cancer or progression of prostate cancer, in a biological sample from a subject, comprising:
(a) providing a biological sample from the subject;
(b) extracting DNA from the biological sample;
(c) detecting the presence of mutations in the ND1, ND2, COX1 or CytB genes of the mtDNA;
(d) determining whether the mutations are associated with normal interpopulation or intrapopulation variations, or whether the mutations are associated with presence of a predisposition to prostate cancer, progression toward prostate cancer, prostate cancer or progression of prostate cancer, and;
(e) repeating steps (a) to (d).

20. The method of claim 19 wherein the biological sample is from a tissue that is chosen from normal tissue, distant benign tissue, adjacent benign tissue, diseased tissue and malignant tissue.

21. The method of claim 20 wherein the step of detecting the presence of mutations in the ND1, ND2, COX1 and CytB genes of mitochondrial DNA comprises at least one of:
(a) comparing the mtDNA of the biological sample to the database of claim 18;
(b) comparing the mtDNA of the biological sample to a sample of mtDNA from non-involved tissue or non-involved bodily fluid from the subject;
(c) comparing the mtDNA of the biological sample to a sample of mtDNA from a maternal relative of the subject;
(d) determining total mutation load of the biological sample; and
(e) identifying the mutations.

22. The method of any of claims 19 to 21 wherein the mutations are synonymous or non-synonymous.

23. The method of claim 19 for monitoring the progression toward prostate cancer or progression of prostate cancer further comprising monitoring the subject at successive time periods for an increase in mutations in ND1, ND2, COX1 or CytB, or an increase in the number of mitochondrial genomes having mutations in ND1, ND2, COX1 or CytB.

24. An isolated mitochondrial ND1 nucleic acid molecule comprising at least one synonymous or non-synonymous mutation for use in detecting the progression toward prostate cancer or the presence of prostate cancer in a subject.

25. An isolated mitochondrial ND2 nucleic acid molecule comprising at least one synonymous or non-synonymous mutation for use in detecting the progression toward prostate cancer or the presence of prostate cancer in a subject.

26. An isolated mitochondrial COX1 nucleic acid molecule comprising at least one synonymous or non-synonymous mutation for use in detecting the progression toward prostate cancer or the presence of prostate cancer in a subject.

27. An isolated mitochondrial CytB nucleic acid molecule comprising at least one synonymous or non-synonymous mutation for use in detecting the progression toward prostate cancer or the presence of prostate cancer in a subject.
